SEQUENCE LISTING (88 sequences)

SEQ ID NO: 1 - protein, 154 aa, Bos taurus
  REGION 1..154: note = MISC_FEATURE - Bovine myoglobin P02192
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 2 - protein, 154 aa, Gallus gallus
  REGION 1..154: note = MISC_FEATURE - Chicken myoglobin P02197
MGLSDQEWQQVLTIWGKVEADIAGHGHEVLMRLFHDHPETLDRFDKFKGLKTPDQMKGSEDLKKHGATVLTQLGKILKQKGNHESELKPLAQTHATKHKIPVKYLEFISEVIIKVIAEKHAADFGADSQAAMKKALELFRNDMASKYKEFGFQG

SEQ ID NO: 3 - protein, 154 aa, Sus scrofa
  REGION 1..154: note = MISC_FEATURE - Swine myoglobin P02189
MGLSDGEWQLVLNVWGKVEADVAGHGQEVLIRLFKGHPETLEKFDKFKHLKSEDEMKASEDLKKHGNTVLTALGGILKKKGHHEAELTPLAQSHATKHKIPVKYLEFISEAIIQVLQSKHPGDFGADAQGAMSKALELFRNDMAAKYKELGFQG

SEQ ID NO: 4 - protein, 142 aa, Bos taurus
  REGION 1..142: note = MISC_FEATURE - Bovine alpha hemoglobin P01966
MVLSAADKGNVKAAWGKVGGHAAEYGAEALERMFLSFPTTKTYFPHFDLSHGSAQVKGHGAKVAAALTKAVEHLDDLPGALSELSDLHAHKLRVDPVNFKLLSHSLLVTLASHLPSDFTPAVHASLDKFLANVSTVLTSKYR

SEQ ID NO: 5 - protein, 142 aa, Gallus gallus
  REGION 1..142: note = MISC_FEATURE - Chicken hemoglobin P01994
MVLSAADKNNVKGIFTKIAGHAEEYGAETLERMFTTYPPTKTYFPHFDLSHGSAQIKGHGKKVVAALIEAANHIDDIAGTLSKLSDLHAHKLRVDPVNFKLLGQCFLVVVAIHHPAALTPEVHASLDKFLCAVGTVLTAKYR

SEQ ID NO: 6 - protein, 142 aa, Sus scrofa
  REGION 1..142: note = MISC_FEATURE - Swine hemoglobin P01965
MVLSAADKANVKAAWGKVGGQAGAHGAEALERMFLGFPTTKTYFPHFNLSHGSDQVKAHGQKVADALTKAVGHLDDLPGALSALSDLHAHKLRVDPVNFKLLSHCLLVTLAAHHPDDFNPSVHASLDKFLANVSTVLTSKYR

SEQ ID NO: 7 - protein, 377 aa, Bos taurus
  REGION 1..377: note = MISC_FEATURE - Bovine alpha actin P68138
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 8 - protein, 377 aa, Gallus gallus; identical to SEQ ID NO: 7
  REGION 1..377: note = MISC_FEATURE - Chicken actin P68139

SEQ ID NO: 9 - protein, 377 aa, Sus scrofa; identical to SEQ ID NO: 7
  REGION 1..377: note = MISC_FEATURE - Swine actin P68137

SEQ ID NO: 10 - protein, 214 aa, Bos taurus
  REGION 1..214: note = MISC_FEATURE - Bovine alpha-S1-casein NP_851372.1
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 11 - protein, 222 aa, Bos taurus
  REGION 1..222: note = MISC_FEATURE - Bovine alpha-S2-casein NP_776953.1
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 12 - protein, 224 aa, Bos taurus
  REGION 1..224: note = MISC_FEATURE - Bovine beta-casein AAA30480.1
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 13 - protein, 190 aa, Bos taurus
  REGION 1..190: note = MISC_FEATURE - Bovine kappa-casein NP_776719.1
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 14 - protein, 381 aa, Bos taurus
  REGION 1..381: note = Bovine chymosyn BP00794; note = MISC_FEATURE - Bovine chymosin B P00794
MRCLVVLLAVFALSQGAEITRIPLYKGKSLRKALKEHGLLEDFLQKQQYGISSKYSGFGEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSGILNIQQAIGATQNQYGEFDIDCDNLSYMPTVVFEINGKMYPLTPSAYTSQDQGFCTSGFQSENHSQKWILGDVFIREYYSVFDRANNLVGLAKAI

SEQ ID NO: 15 - protein, 199 aa, synthetic construct
  REGION 1..199: note = Synthetic construct, Alpha-S1-casein_CasA1; trunc_NP_851372.1
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 16 - protein, 207 aa, synthetic construct
  REGION 1..207: note = Synthetic construct, Alpha-S2-casein_CasA2; trunc_NP_776953.1
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 17 - protein, 209 aa, synthetic construct
  REGION 1..209: note = Synthetic construct, Beta-casein_CasB; trunc_AAA30480.1
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 18 - protein, 169 aa, synthetic construct
  REGION 1..169: note = Synthetic construct, Kappa-casein_CasK; trunc_NP_776719.1
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 19 - protein, 365 aa, synthetic construct
  REGION 1..365: note = Synthetic construct, Chymosin B_CymB; trunc_P00794
AEITRIPLYKGKSLRKALKEHGLLEDFLQKQQYGISSKYSGFGEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSGILNIQQAIGATQNQYGEFDIDCDNLSYMPTVVFEINGKMYPLTPSAYTSQDQGFCTSGFQSENHSQKWILGDVFIREYYSVFDRANNLVGLAKAI

SEQ ID NO: 20 - protein, 153 aa, synthetic construct
  REGION 1..153: note = Synthetic construct, Myoglobin_MYG; trunc_P02192
GLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 21 - protein, 141 aa, synthetic construct
  REGION 1..141: note = Synthetic construct, Alpha hemoglobin_HBA; trunc_P01966
VLSAADKGNVKAAWGKVGGHAAEYGAEALERMFLSFPTTKTYFPHFDLSHGSAQVKGHGAKVAAALTKAVEHLDDLPGALSELSDLHAHKLRVDPVNFKLLSHSLLVTLASHLPSDFTPAVHASLDKFLANVSTVLTSKYR

SEQ ID NO: 22 - protein, 376 aa, synthetic construct
  REGION 1..376: note = Synthetic construct, Alpha actin _ACTA; trunc_68138
CDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 23 - protein, 19 aa, synthetic construct
  REGION 1..19: note = Synthetic construct, m Z27 signal peptide
MRVLLVALALLALAASATS

SEQ ID NO: 24 - DNA, 57 bp, synthetic construct
  misc_feature 1..57: note = Synthetic construct, mZ27 signal peptide coding seq
  source 1..57: mol_type = other DNA
atgagggtgttgctcgttgccctcgctctcctggctctcgctgcgagcgccaccagc

SEQ ID NO: 25 - protein, 4 aa, synthetic construct
  REGION 1..4: note = Synthetic construct, KDEL signal peptide
KDEL

SEQ ID NO: 26 - DNA, 12 bp, synthetic construct
  misc_feature 1..12: note = Synthetic construct, KDEL coding sequence
  source 1..12: mol_type = other DNA
aaggacgagctg

SEQ ID NO: 27 - DNA, 597 bp, synthetic construct
  misc_feature 1..597: note = Synthetic construct, Alpha-S1-casein CasA1; trunc_coding seq
  source 1..597: mol_type = other DNA
cggcccaagcatcctataaaacatcaaggtttgcctcaagaagttttgaatgaaaatttgctccgcttcttcgtcgccccctttccggaggtttttgggaaagagaaggtaaatgagctcagcaaggacattggttctgaaagtacagaagaccaggcgatggaggatatcaagcagatggaagcagagtccatatcttcttcagaggagatcgtccctaacagcgtggagcaaaagcacatccagaaggaggacgtgccgtcggagcgctacctgggatatttagagcagctgctccgtctcaagaagtacaaggtgccacaactggaaattgttcccaacagtgctgaagaaagacttcactccatgaaagaaggcatccacgcgcagcagaaagaaccaatgattggcgtcaaccaagagcttgcctacttctatcctgagctgttcaggcagttctaccagctggatgcttatccgtccggcgcgtggtactacgtgccgctgggcacgcagtatactgatgcaccatcattttcggacatcccaaatccaattgggagcgagaactccgagaaaaccaccatgccgctgtgg

SEQ ID NO: 28 - DNA, 621 bp, synthetic construct
  misc_feature 1..621: note = Synthetic construct, Alpha-S2-casein CasA2; trunc_coding seq
  source 1..621: mol_type = other DNA
aaaaacacaatggagcatgttagttcttcggaggagagcataatttcacaagaaacatacaagcaggagaagaacatggccatcaacccgtcgaaagagaacctctgtagtactttctgcaaggaagttgtgaggaatgctaatgaagaggagtacagcattggttcatcctctgaggaaagcgcggaggttgctacagaagaggtcaagatcactgttgatgacaagcactaccaaaaggcattgaatgagataaatcagttctaccaaaaatttccgcagtatttgcagtacctgtatcaaggccccatcgtgctgaatccgtgggaccaggtgaagcgcaacgcagtgccgatcacacctactcttaaccgtgagcagctctccacgtctgaagaaaatagcaaaaaaacagtagacatggaatcaactgaggtcttcaccaaaaagaccaagttaaccgaggaggaaaagaatagactgaacttcctcaagaaaatttcccagaggtatcaaaagtttgcgctgccacaatatcttaaaacggtgtaccagcaccagaaggccatgaagccctggattcagccaaagacgaaggtcattccttatgtccgctacctg

SEQ ID NO: 29 - DNA, 627 bp, synthetic construct
  misc_feature 1..627: note = Synthetic construct, Beta-casein CasB; trunc_coding seq
  source 1..627: mol_type = other DNA
agagaactggaggagctgaatgtgcccggcgagattgttgagagcttgtcgtcgtcagaagaatccatcacccggatcaacaagaagattgagaagttccagtctgaagagcagcagcaaacagaggatgagcttcaggacaagatacatccattcgcccagacacaatctctcgtctacccgttcccaggccccatcccaaacagtcttcctcaaaatattccgccgctgacgcagacccccgtggttgttcctcccttcctgcagccggaggtgatgggcgtcagcaaggtgaaagaagcgatggcccccaagcacaaggagatgccgtttccaaaatatccagttgaaccttttactgaatctcaaagtttgacgttaactgatgtggagaacctccacctccctcttccattgctacagtcatggatgcatcaacctcaccagcctttgccgccgaccgtcatgttccctccacagagcgtgctgtccctctcccaatcaaaagttctacccgtaccccagaaagctgtgccatatcctcagagggacatgccaatacaagcatttctgctctaccaggagccggtacttggacctgtccgcgggccatttcctatcattgtc

SEQ ID NO: 30 - DNA, 507 bp, synthetic construct
  misc_feature 1..507: note = Synthetic construct, Kappa-casein CasK; trunc coding seq
  source 1..507: mol_type = other DNA
caagaacaaaaccaggagcagccgatacgttgtgagaaggacgagaggtttttttctgataaaattgccaagtacatccctattcagtacgttctttcgcgctacccgtcctacgggctgaactactatcagcagaagccggttgcactcatcaataatcagttcctcccatatccatattatgccaaaccggccgcagtcagatcaccagcgcagatattgcagtggcaggtcctttccaacacagtgccggctaagagctgccaagctcaaccgacgacaatggcccgccaccctcatccacacctgagcttcatggcgatcccccccaagaaaaatcaagacaagacggaaattccgaccatcaatacaatagcttcaggcgaacctacaagtactcctacaactgaagcggtcgagtcgactgttgcgaccctggaagattctcccgaggtgattgagagccctccagagatcaacaccgtgcaggtgacgagtactgccgta

SEQ ID NO: 31 - DNA, 1095 bp, synthetic construct
  misc_feature 1..1095: note = Synthetic construct, Chymosin B CymB; trunc_coding seq
  source 1..1095: mol_type = other DNA
gcggaaataacccgcatcccgctctacaaggggaaaagtttgcggaaagctctgaaggagcatggactgctggaggatttcctgcagaagcagcaatatggaatatcctcaaagtactctggttttggggaggtggcgtcagttcccttaaccaactaccttgattcacagtacttcgggaagatttacctcggcactcctcctcaagagtttacggttctatttgatactggttcctcggacttctgggttccgtccatctactgcaaatctaatgcttgcaagaaccaccagaggttcgacccgcgaaagagctcgacgtttcaaaatcttggcaaacctctctctatccactacggcaccggctccatgcagggcatactgggttatgatacagtgaccgtcagcaatattgtagacattcaacaaacagtcgggctatctacccaagaacccggtgatgttttcacctatgcagagttcgacggtattctcggcatggcctacccatcgttagctagcgagtatagcataccggtgtttgataacatgatgaatcgccatctcgtcgcgcaggacctgttctccgtgtacatggatagaaatggacaggagtccatgctgactttgggtgctattgatccctcttattatactggctcacttcactgggtgccggtcacggtgcagcagtactggcagttcactgttgattctgttacaatcagcggcgtcgtcgtagcatgtgaaggcggctgccaggcgattcttgacaccggaacaagtaaattggttgggccgagcagcgacatcctcaatatccagcaggccattggagcaacgcagaaccagtatggcgaatttgatattgactgtgacaacttgtcctacatgccaacagtggtgtttgagatcaacggcaagatgtatccacttactccatcggcatatacatctcaagatcaaggcttctgcacgtcaggattccagtcagaaaatcatagtcaaaagtggatcttgggcgacgtgttcatccgtgagtactacagcgtcttcgaccgcgccaacaacctggtagggctcgccaaggccata

SEQ ID NO: 32 - DNA, 459 bp, synthetic construct
  misc_feature 1..459: note = Synthetic construct, Myoglobin MYG; trunc_coding seq
  source 1..459: mol_type = other DNA
gggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatgga

SEQ ID NO: 33 - DNA, 423 bp, synthetic construct
  misc_feature 1..423: note = Synthetic construct, Alpha hemoglobin _HBA; trunc_coding seq
  source 1..423: mol_type = other DNA
gtgctctcggccgccgacaaagggaacgtcaaagcggcgtggggcaaggtcggcggccatgctgctgagtatggtgctgaagctcttgaaaggatgttcttgagttttcctacaacgaaaacctacttcccacattttgatttgagtcatggatcagcacaggtgaagggtcacggggccaaggtggcggccgcgctcacaaaagcagtggagcacctcgacgaccttcctggagcattatcggagctgagcgacctgcacgcgcacaagctaagagttgatccagtcaacttcaagctgctgtcccacagcctgctggtaacgctggccagccatttgccgtctgattttacccccgccgtccatgcaagcctagacaagttcctcgctaatgtttctactgttcttacttccaagtaccgc

SEQ ID NO: 34 - DNA, 1128 bp, synthetic construct
  misc_feature 1..1128: note = Synthetic construct, Alpha actin _ACTA; trunc_coding seq
  source 1..1128: mol_type = other DNA
tgcgacgaggatgaaaccaccgctcttgtttgtgacaacggctcgggcctcgtgaaggccgggtttgctggtgatgatgcaccccgcgcggtattcccctctattgtcgggcggccgcgccaccagggcgtgatggttggcatgggtcaaaaggatagctacgtgggtgatgaggcgcagagtaaacgtggcatactgaccctaaagtacccaattgaacatggcatcatcaccaactgggacgacatggaaaaaatttggcaccacacgttctacaatgagcttcgagtggccccggaggagcatccaactctcctaactgaagcaccgctcaaccccaaggcaaatagggagaagatgacacaaataatgtttgagacattcaacgtgcctgcgatgtatgtggctatacaagctgttttgtcactgtacgccagtggaagaacaactggaattgtactggactcaggtgacggcgtcacccacaatgttcctatatatgaaggctatgcactgccgcacgccatcatgcgccttgatcttgctggacgcgacctcaccgactacctaatgaagattttgacagaaagaggatacagctttgtcactacggccgagagggagatcgtgagggatatcaaagaaaagctctgctacgttgcgctcgacttcgagaacgagatggccacggcggcgagctcctctagtctggagaaatcttatgaactgcctgatgggcaggtgatcactataggaaatgaaagatttcggtgccccgagaccttgtttcaacctagtttcatagggatggagtcagcgggcattcatgaaacgacttacaattccatcatgaaatgtgatatcgatattaggaaagacctgtatgccaacaatgtcatgtcggggggtacaacgatgtaccctgggattgctgataggatgcagaaggagataactgctttggcaccatctaccatgaagatcaagattatcgccccgccagaacgtaaatattctgtctggattggagggtcgattcttgccagcctgtccacattccagcagatgtggatcacaaagcaggagtacgacgaggcgggcccgtccatcgttcatcggaagtgcttc

SEQ ID NO: 35 - protein, 222 aa, synthetic construct
  REGION 1..222: note = Synthetic construct, Alpha-S1-casein (CasA1; opt)
MRVLLVALALLALAASATSRPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWKDEL

SEQ ID NO: 36 - protein, 230 aa, synthetic construct
  REGION 1..230: note = Synthetic construct, Alpha-S2-casein (CasA2; opt)
MRVLLVALALLALAASATSKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYLKDEL

SEQ ID NO: 37 - protein, 232 aa, synthetic construct
  REGION 1..232: note = Synthetic construct, Beta-casein (CasB; opt)
MRVLLVALALLALAASATSRELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVKDEL

SEQ ID NO: 38 - protein, 192 aa, synthetic construct
  REGION 1..192: note = Synthetic construct, Kappa-casein (CasK; opt)
MRVLLVALALLALAASATSQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAVKDEL

SEQ ID NO: 39 - protein, 388 aa, synthetic construct
  REGION 1..388: note = Synthetic construct, Chymosin B (CymB; opt)
MRVLLVALALLALAASATSAEITRIPLYKGKSLRKALKEHGLLEDFLQKQQYGISSKYSGFGEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSGILNIQQAIGATQNQYGEFDIDCDNLSYMPTVVFEINGKMYPLTPSAYTSQDQGFCTSGFQSENHSQKWILGDVFIREYYSVFDRANNLVGLAKAIKDEL

SEQ ID NO: 40 - protein, 176 aa, synthetic construct
  REGION 1..176: note = Synthetic construct, Myoglobin (MYG; opt)
MRVLLVALALLALAASATSGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHGKDEL

SEQ ID NO: 41 - protein, 164 aa, synthetic construct
  REGION 1..164: note = Synthetic construct, Alpha hemoglobin (HBA; opt)
MRVLLVALALLALAASATSVLSAADKGNVKAAWGKVGGHAAEYGAEALERMFLSFPTTKTYFPHFDLSHGSAQVKGHGAKVAAALTKAVEHLDDLPGALSELSDLHAHKLRVDPVNFKLLSHSLLVTLASHLPSDFTPAVHASLDKFLANVSTVLTSKYRKDEL

SEQ ID NO: 42 - protein, 399 aa, synthetic construct
  REGION 1..399: note = Synthetic construct, Alpha actin (ACTA; opt)
MRVLLVALALLALAASATSCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCFKDEL

SEQ ID NO: 43 - DNA, 3433 bp, synthetic construct
  misc_feature 1..3433: note = Synthetic construct, pAG5009
  source 1..3433: mol_type = other DNA
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagcgcggaaataacccgcatcccgctctacaaggggaaaagtttgcggaaagctctgaaggagcatggactgctggaggatttcctgcagaagcagcaatatggaatatcctcaaagtactctggttttggggaggtggcgtcagttcccttaaccaactaccttgattcacagtacttcgggaagatttacctcggcactcctcctcaagagtttacggttctatttgatactggttcctcggacttctgggttccgtccatctactgcaaatctaatgcttgcaagaaccaccagaggttcgacccgcgaaagagctcgacgtttcaaaatcttggcaaacctctctctatccactacggcaccggctccatgcagggcatactgggttatgatacagtgaccgtcagcaatattgtagacattcaacaaacagtcgggctatctacccaagaacccggtgatgttttcacctatgcagagttcgacggtattctcggcatggcctacccatcgttagctagcgagtatagcataccggtgtttgataacatgatgaatcgccatctcgtcgcgcaggacctgttctccgtgtacatggatagaaatggacaggagtccatgctgactttgggtgctattgatccctcttattatactggctcacttcactgggtgccggtcacggtgcagcagtactggcagttcactgttgattctgttacaatcagcggcgtcgtcgtagcatgtgaaggcggctgccaggcgattcttgacaccggaacaagtaaattggttgggccgagcagcgacatcctcaatatccagcaggccattggagcaacgcagaaccagtatggcgaatttgatattgactgtgacaacttgtcctacatgccaacagtggtgtttgagatcaacggcaagatgtatccacttactccatcggcatatacatctcaagatcaaggcttctgcacgtcaggattccagtcagaaaatcatagtcaaaagtggatcttgggcgacgtgttcatccgtgagtactacagcgtcttcgaccgcgccaacaacctggtagggctcgccaaggccataaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 44 - DNA, 2935 bp, synthetic construct
  misc_feature 1..2935: note = Synthetic construct, pAG5011
  source 1..2935: mol_type = other DNA
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagccggcccaagcatcctataaaacatcaaggtttgcctcaagaagttttgaatgaaaatttgctccgcttcttcgtcgccccctttccggaggtttttgggaaagagaaggtaaatgagctcagcaaggacattggttctgaaagtacagaagaccaggcgatggaggatatcaagcagatggaagcagagtccatatcttcttcagaggagatcgtccctaacagcgtggagcaaaagcacatccagaaggaggacgtgccgtcggagcgctacctgggatatttagagcagctgctccgtctcaagaagtacaaggtgccacaactggaaattgttcccaacagtgctgaagaaagacttcactccatgaaagaaggcatccacgcgcagcagaaagaaccaatgattggcgtcaaccaagagcttgcctacttctatcctgagctgttcaggcagttctaccagctggatgcttatccgtccggcgcgtggtactacgtgccgctgggcacgcagtatactgatgcaccatcattttcggacatcccaaatccaattgggagcgagaactccgagaaaaccaccatgccgctgtggaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 45 - DNA, 2464 bp, synthetic construct
  misc_feature 1..2464: note = Synthetic construct, pAG5012
  source 1..2464: mol_type = other DNA
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatccaccatgagggtgttgctcgttgccctcgctctcctggctctcgctgcgagcgccaccagcagagaactggaggagctgaatgtgcccggcgagattgttgagagcttgtcgtcgtcagaagaatccatcacccggatcaacaagaagattgagaagttccagtctgaagagcagcagcaaacagaggatgagcttcaggacaagatacatccattcgcccagacacaatctctcgtctacccgttcccaggccccatcccaaacagtcttcctcaaaatattccgccgctgacgcagacccccgtggttgttcctcccttcctgcagccggaggtgatgggcgtcagcaaggtgaaagaagcgatggcccccaagcacaaggagatgccgtttccaaaatatccagttgaaccttttactgaatctcaaagtttgacgttaactgatgtggagaacctccacctccctcttccattgctacagtcatggatgcatcaacctcaccagcctttgccgccgaccgtcatgttccctccacagagcgtgctgtccctctcccaatcaaaagttctacccgtaccccagaaagctgtgccatatcctcagagggacatgccaatacaagcatttctgctctaccaggagccggtacttggacctgtccgcgggccatttcctatcattgtcaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 46 - DNA, 2233 bp, synthetic construct
  misc_feature 1..2233: note = Synthetic construct, pAG5013
  source 1..2233: mol_type = other DNA
aaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatccaccatgagggtgttgctcgttgccctcgctctcctggctctcgctgcgagcgccaccagccaagaacaaaaccaggagcagccgatacgttgtgagaaggacgagaggtttttttctgataaaattgccaagtacatccctattcagtacgttctttcgcgctacccgtcctacgggctgaactactatcagcagaagccggttgcactcatcaataatcagttcctcccatatccatattatgccaaaccggccgcagtcagatcaccagcgcagatattgcagtggcaggtcctttccaacacagtgccggctaagagctgccaagctcaaccgacgacaatggcccgccaccctcatccacacctgagcttcatggcgatcccccccaagaaaaatcaagacaagacggaaattccgaccatcaatacaatagcttcaggcgaacctacaagtactcctacaactgaagcggtcgagtcgactgttgcgaccctggaagattctcccgaggtgattgagagccctccagagatcaacaccgtgcaggtgacgagtactgccgtaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 47 - DNA, 2347 bp, synthetic construct
  misc_feature 1..2347: note = Synthetic construct, pAG5014
  source 1..2347: mol_type = other DNA
aaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatccaccatgagggtgttgctcgttgccctcgctctcctggctctcgctgcgagcgccaccagcaaaaacacaatggagcatgttagttcttcggaggagagcataatttcacaagaaacatacaagcaggagaagaacatggccatcaacccgtcgaaagagaacctctgtagtactttctgcaaggaagttgtgaggaatgctaatgaagaggagtacagcattggttcatcctctgaggaaagcgcggaggttgctacagaagaggtcaagatcactgttgatgacaagcactaccaaaaggcattgaatgagataaatcagttctaccaaaaatttccgcagtatttgcagtacctgtatcaaggccccatcgtgctgaatccgtgggaccaggtgaagcgcaacgcagtgccgatcacacctactcttaaccgtgagcagctctccacgtctgaagaaaatagcaaaaaaacagtagacatggaatcaactgaggtcttcaccaaaaagaccaagttaaccgaggaggaaaagaatagactgaacttcctcaagaaaatttcccagaggtatcaaaagtttgcgctgccacaatatcttaaaacggtgtaccagcaccagaaggccatgaagccctggattcagccaaagacgaaggtcattccttatgtccgctacctgaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 48 - DNA, 2797 bp, synthetic construct
  misc_feature 1..2797: note = Synthetic construct, pAG5016, pAG5020 (2nd cassette)
  source 1..2797: mol_type = other DNA
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 49 - DNA, 2761 bp, synthetic construct
  misc_feature 1..2761: note = Synthetic construct, pAG5017
  source 1..2761: mol_type = other DNA
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagcgtgctctcggccgccgacaaagggaacgtcaaagcggcgtggggcaaggtcggcggccatgctgctgagtatggtgctgaagctcttgaaaggatgttcttgagttttcctacaacgaaaacctacttcccacattttgatttgagtcatggatcagcacaggtgaagggtcacggggccaaggtggcggccgcgctcacaaaagcagtggagcacctcgacgaccttcctggagcattatcggagctgagcgacctgcacgcgcacaagctaagagttgatccagtcaacttcaagctgctgtcccacagcctgctggtaacgctggccagccatttgccgtctgattttacccccgccgtccatgcaagcctagacaagttcctcgctaatgtttctactgttcttacttccaagtaccgcaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 50 - DNA, 3466 bp, synthetic construct
  misc_feature 1..3466: note = Synthetic construcr, pAG5018
  source 1..3466: mol_type = other DNA
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagctgcgacgaggatgaaaccaccgctcttgtttgtgacaacggctcgggcctcgtgaaggccgggtttgctggtgatgatgcaccccgcgcggtattcccctctattgtcgggcggccgcgccaccagggcgtgatggttggcatgggtcaaaaggatagctacgtgggtgatgaggcgcagagtaaacgtggcatactgaccctaaagtacccaattgaacatggcatcatcaccaactgggacgacatggaaaaaatttggcaccacacgttctacaatgagcttcgagtggccccggaggagcatccaactctcctaactgaagcaccgctcaaccccaaggcaaatagggagaagatgacacaaataatgtttgagacattcaacgtgcctgcgatgtatgtggctatacaagctgttttgtcactgtacgccagtggaagaacaactggaattgtactggactcaggtgacggcgtcacccacaatgttcctatatatgaaggctatgcactgccgcacgccatcatgcgccttgatcttgctggacgcgacctcaccgactacctaatgaagattttgacagaaagaggatacagctttgtcactacggccgagagggagatcgtgagggatatcaaagaaaagctctgctacgttgcgctcgacttcgagaacgagatggccacggcggcgagctcctctagtctggagaaatcttatgaactgcctgatgggcaggtgatcactataggaaatgaaagatttcggtgccccgagaccttgtttcaacctagtttcatagggatggagtcagcgggcattcatgaaacgacttacaattccatcatgaaatgtgatatcgatattaggaaagacctgtatgccaacaatgtcatgtcggggggtacaacgatgtaccctgggattgctgataggatgcagaaggagataactgctttggcaccatctaccatgaagatcaagattatcgccccgccagaacgtaaatattctgtctggattggagggtcgattcttgccagcctgtccacattccagcagatgtggatcacaaagcaggagtacgacgaggcgggcccgtccatcgttcatcggaagtgcttcaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 51 - DNA, 2296 bp, synthetic construct
  misc_feature 1..2296: note = Synthetic construct, pAG5019
  source 1..2296: mol_type = other DNA
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 52 - DNA, 2200 bp, synthetic construct
  misc_feature 1..2200: note = Synthetic construct, pAG5020 (1st expression cassette)
  source 1..2200: mol_type = other DNA
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggctgcacatagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 53 - DNA, 10247 bp, synthetic construct
  misc_feature 1..10247: note = Synthetic construct, pAG4500 vector
  source 1..10247: mol_type = other DNA
catcaatcccgggtattcatcctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggaattcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagatccagatctaaaccatgcagaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcgagctcgaattaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaacctgatcatgagcggagaattaagggagtcacgttatgacccccgccgatgacgcgggacaagccgttttacgtttggaactgacagaaccgcaacgttgaaggagccactcagcctaagcggccgcattggacttaattaagtgaggccggccaagcgtcgatttaaatgtaccacatggcgcgccaactatcatgcgatcgcttcatgtctaactcgagttactggtacgtaccaaatccatggaatcaaggtac

SEQ ID NO: 54 - DNA, 106 bp, synthetic construct
  misc_feature 1..106: note = Synthetic construct, pAG4500 10100-10205 in SEQ ID NO: 53
  source 1..106: mol_type = other DNA
ctcagcctaagcggccgcattggacttaattaagtgaggccggccaagcgtcgatttaaatgtaccacatggcgcgccaactatcatgcgatcgcttcatgtctaa

SEQ ID NO: 55 - DNA, 105 bp, synthetic construct
  misc_feature 1..105: note = Synthetic construct, pAG4003 10100-10205 in SEQ ID NO: 53
  source 1..105: mol_type = other DNA
ctcagcttaagcggccgcattggacttaattaagtgaggccggccaagcgtcgatttaaatgtaccacatggcgcgccaactatcatgcgatcgcttaagtctaa

SEQ ID NO: 56 - DNA, 5003 bp, synthetic construct
  misc_feature 1..5003: note = Synthetic construct, pAG5020 (two cassettes)
  source 1..5003: mol_type = other DNA
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggctgcacatagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagggtacctccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 57 - protein, 6 aa, synthetic construct
  REGION 1..6: note = SEKDEL signal peptide
SEKDEL

SEQ ID NO: 58 - protein, 154 aa, Bos taurus
  VARIANT 1..154: note = BtMYGm mutated variant
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFHSDAIIHVLHAKHPSDFGADAQAAMSKALELFRNDMAAQYKVLGFHG

SEQ ID NO: 59 - protein, 454 aa, Hordeum vulgare
  VARIANT 1..454: note = Barley glutamyl-tRNA reductase HvGluTRm truncated
EKSSIAVIGLSVHTAPVEMREKLAVAEELWPRAISELTSLNHIEEAAVLSTCNRMEIYVVALSWNRGIREVVDWMSKKSGIPASELREHLFMLRDSDATRHLFEVSAGLDSLVLGEGQILAQVKQVVRNGQNSGGLGKNIDRMFKDAITAGKRVRCETNISAGAVSVSSAAVELAMMKLPKSECLSARMLLIGAGKMGKLVVKHLIAKGCKKVVVVNRSVERVDAIREEMKDIEIVYRPLTEMYEAAADADVVFTSTASESLLFTKEHAEVLPPISLAMGGVRLFVDISVPRNVGACLSEVEHARVYNVDDLKEVVEANKEDRVRKAMEAQTIITQELKRFEAWRDSLETVPTIKKLRSYADRIRASELEKCLQKIGEDNLNKKMRRSIEELSTGIVNKLLHGPLQHLRCDGSDSRTLDETLENMHALNRMFSLDTEKAVLEQKIKAKVEKTQS

SEQ ID NO: 60 - protein, 51 aa, Zea mays
  REGION 1..51: note = Maize glutamyl tRNA reductase signal seq ZmGluRTss
MMASTAPAASTAVAFGAAATPRGPASASAALCARVPAAAGGMRRSGVAVRC

SEQ ID NO: 61 - protein, 484 aa, Zea mays
  REGION 1..484: note = Maize ferrochelatese ZmFC1A
MSSSGPSPATGIHASPPLGLLPATGTHHTRSWGKTTSTSFTGSTTKHEQSLHGNVKPLQLAANESSRLAYRSPALKNQWNLPASSSSTNVVTTFDDNEHVSSSVIEEKVGVLLLNLGGPETLDDVQPFLFNLFADPDIIRLPRLFRFLQRPLAKLISTFRAPKSKEGYASIGGGSPLRKITDEQANALKIALEKKKLNANIYVGMRYWYPFTEEAIDQIKKDKITKLVVLPLYPQYSISTSGSSIRVLQDIVKEDSYFSGLPISIIESWYQRDGYVKSMSDLIEKELSAFSNPEEVMIFFSAHGVPLTYVEDAGDPYRDQMEDCIALIMGELRSRGILNSHTLAYQSRVGPVQWLKPYTDEVLVELGQKGVKSLLAVPVSFVSEHIETLEEIDMEYKELALESGIKNWGRVPALGCTSTFISDLADAVVEALPSASALATRKPDDIDSSMDLTHYLTKMLFGSILAFVLLLSPRLVSAFRSTML

SEQ ID NO: 62 - protein, 19 aa, Zea mays
  REGION 1..19: note = Signal sequence maize Al9
MAKLLSLFLALSFVAAMFA

SEQ ID NO: 63 - protein, 24 aa, Glycine max
  PEPTIDE 1..24: note = Signal sequence of alpha subunit beta-conglycinin
MMRARFPLLLLGVVFLASVSVSFG

SEQ ID NO: 64 - DNA, 2797 bp, synthetic construct
  source 1..2797: mol_type = other DNA
  misc_feature 1..2797: note = pAG5024
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttccacagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 65 - DNA, 2296 bp, synthetic construct
  source 1..2296: mol_type = other DNA
  misc_feature 1..2296: note = pAG5025
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttccacagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 66 - DNA, 5003 bp, synthetic construct
  source 1..5003: mol_type = other DNA
  misc_feature 1..5003: note = pAG5026
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagctggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttccacagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggctgcacatagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagggtacctccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttccacagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 67 - DNA, 3784 bp, synthetic construct
  source 1..3784: mol_type = other DNA
  misc_feature 1..3784: note = pAG5027
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgatggcgagcacggctccagcggcctccaccgccgtggcattcggggcagccgccacgccgcggggaccggcgtcggcgtcggcggcgctctgcgcgagggtgcctgccgctgccggcggcatgcgtcgctccggggtggccgtgcggtgcgagaaatctagcatcgctgtcatcgggctgtctgtccacactgcccctgtcgagatgcgggagaaactagcggtggccgaggagctgtggccgagggccattagtgagttgacgtccttaaatcacatcgaagaagcagccgtgttatctacatgcaacagaatggaaatttatgttgttgcgctgtcgtggaaccggggcattcgcgaggtcgtggactggatgtccaagaaaagcggcatcccggcttctgagttgcgtgagcatctcttcatgctgcgcgacagtgatgccacaagacacttgtttgaagtatctgcaggcctcgattccctcgttttgggagagggacagatcctggctcaagtgaagcaggtggtcaggaatggccagaacagtggcggtttaggtaaaaatattgatcgaatgttcaaagatgctattactgctgggaagcgagttcgatgtgaaacaaatatctccgcgggcgcggtatccgtgtcctcagcggccgtggaacttgcaatgatgaaacttccaaaatcagagtgcctatcggcccgtatgctccttataggagccggaaaaatgggcaagctggtcgtcaagcatctgattgcaaaggggtgcaagaaggttgttgtggtcaaccggtcagtggagagggttgacgccatcagggaggaaatgaaagacattgaaatcgtgtacaggcctctcaccgagatgtacgaagctgcggccgacgctgacgtagtcttcacgtcaactgcatcagagtcgctgctttttaccaaggagcacgccgaggtgttgcccccgatatcgctcgcgatgggcggtgtccggctgttcgtcgacataagcgtgccgcggaatgtgggtgcttgtcttagcgaggttgagcacgcccgcgtgtacaatgttgatgatctgaaggaagtcgttgaagccaacaaggaagaccgtgtaaggaaggcaatggaggcgcagactatcatcacccaagaactcaagaggttcgaggcctggcgcgattctcttgaaactgtgccgaccattaagaagctgcgctcatatgctgacagaatacgtgcttcggaattggagaagtgcctccaaaagataggcgaagataacctcaacaagaagatgaggagaagtattgaggagctgagcaccgggattgtcaataaacttctacatggtccattgcagcacctccgctgtgacggctctgattccagaacactggacgagacacttgaaaacatgcacgcgctaaatcgcatgtttagcctcgatacggagaaagcggttcttgagcaaaaaataaaggcgaaggtagaaaagacccagagctgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 68 - DNA, 3109 bp, synthetic construct
  source 1..3109: mol_type = other DNA
  misc_feature 1..3109: note = pAG5028
aaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatccaccatgtcttcgtcgggcccctccccggcgacgggaatccacgcgtcgccgccgttgggccttttgccggcgacgggaacccatcacaccaggtcatggggcaaaacaacctccacaagttttactggttctaccaccaaacatgagcagagcttgcatggaaatgttaagccgttgcaattggcggcaaatgaatcctctcgtttggcttacagaagtccagcacttaaaaaccagtggaatcttcctgctagttcttcctccactaatgtggttaccacctttgatgataacgaacacgtgtcttccagtgttattgaagaaaaagttggagtactgttattaaaccttggtggtccagagacacttgacgatgttcaaccatttttattcaacctatttgctgatccagatatcattcgactccctagactcttcaggtttcttcaaagaccactggccaaacttatttctacttttagagctcctaagagtaaagaagggtatgcttcaattggtggtgggtcgccgttaaggaaaattactgatgaacaggcgaatgctttgaagattgccctggaaaagaaaaaattgaacgcaaacatatatgttgggatgcggtattggtaccctttcacagaagaggccattgatcaaattaaaaaggataagattaccaagctcgttgttcttcccctttaccctcagtactccatatcaacaagtgggtcaagcattcgtgttctccaagacattgtcaaggaagattcatatttttctggtttgccaatttccattattgaatcatggtaccaacgagatggctatgtgaaatcaatgtctgacctaattgaaaaggagctctccgccttctccaatcctgaagaggttatgatattcttcagtgcacatggtgtgccacttacctatgttgaggatgctggagatccttacagagatcagatggaggattgtattgctttgatcatgggggagttaagatcaagaggaatcttaaatagtcacactttggcgtaccagagtcgggtggggccagttcaatggctgaagccatatactgatgaagttttagtagaacttggtcaaaagggtgtgaagagcctcctggctgttccagtaagctttgtgagtgagcacatcgagacattggaagaaattgacatggagtacaaggagttggctctggaatcaggcatcaagaactggggtcgggttcctgctcttggatgcacttcaacattcatctccgaccttgcagatgctgtcgtcgaagccctgccctctgcttcagcgctcgcgaccagaaagcctgacgacatcgattccagcatggacctgacgcattacctcaccaagatgctgtttggctcaatcttggcatttgtcctgctgctatcaccaaggctagtttccgccttccggagcaccatgctttaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 69 - DNA, 6899 bp, synthetic construct
  source 1..6899: mol_type = other DNA
  misc_feature 1..6899: note = pAG5029
aaagtaatcatattattttatgtgtgaatcttctttactttttcatttgattatgattatgaaggtatgaccttcataaccttcgtccgaaatccattatatccaaaggaaaataatgcttcgaaggacgaaggattttgatatttaacattttatgttgccttgttcttaattcatagcatttgagaacaagtccccaacaccaatctttatctttactatattaaagcaccagttcaacgatcgtctcgtgtcaatattattaaaaaactcctacatttctttataatcaacccgcactcttataatctcttctcttactactataataagagagtttatgtacaaaataaggtgaaattatgtataagtgttctggaccttggttgttggctcatattcacacaacctaatcaatagaaaacatatgttttattaaaacaaaatttatcatatatatatatatatatatatatatatatatatatatatataatataaaccgtagcaatgcacaggcatatgactagtggcaacttaataccatgtgtgtattaagatgaataagaggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggaacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaccggatccaccatgtcttcgtcgggcccctccccggcgacgggaatccacgcgtcgccgccgttgggccttttgccggcgacgggaacccatcacaccaggtcatggggcaaaacaacctccacaagttttactggttctaccaccaaacatgagcagagcttgcatggaaatgttaagccgttgcaattggcggcaaatgaatcctctcgtttggcttacagaagtccagcacttaaaaaccagtggaatcttcctgctagttcttcctccactaatgtggttaccacctttgatgataacgaacacgtgtcttccagtgttattgaagaaaaagttggagtactgttattaaaccttggtggtccagagacacttgacgatgttcaaccatttttattcaacctatttgctgatccagatatcattcgactccctagactcttcaggtttcttcaaagaccactggccaaacttatttctacttttagagctcctaagagtaaagaagggtatgcttcaattggtggtgggtcgccgttaaggaaaattactgatgaacaggcgaatgctttgaagattgccctggaaaagaaaaaattgaacgcaaacatatatgttgggatgcggtattggtaccctttcacagaagaggccattgatcaaattaaaaaggataagattaccaagctcgttgttcttcccctttaccctcagtactccatatcaacaagtgggtcaagcattcgtgttctccaagacattgtcaaggaagattcatatttttctggtttgccaatttccattattgaatcatggtaccaacgagatggctatgtgaaatcaatgtctgacctaattgaaaaggagctctccgccttctccaatcctgaagaggttatgatattcttcagtgcacatggtgtgccacttacctatgttgaggatgctggagatccttacagagatcagatggaggattgtattgctttgatcatgggggagttaagatcaagaggaatcttaaatagtcacactttggcgtaccagagtcgggtggggccagttcaatggctgaagccatatactgatgaagttttagtagaacttggtcaaaagggtgtgaagagcctcctggctgttccagtaagctttgtgagtgagcacatcgagacattggaagaaattgacatggagtacaaggagttggctctggaatcaggcatcaagaactggggtcgggttcctgctcttggatgcacttcaacattcatctccgaccttgcagatgctgtcgtcgaagccctgccctctgcttcagcgctcgcgaccagaaagcctgacgacatcgattccagcatggacctgacgcattacctcaccaagatgctgtttggctcaatcttggcatttgtcctgctgctatcaccaaggctagtttccgccttccggagcaccatgctttaacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtacctccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacggatccaccatgatggcgagcacggctccagcggcctccaccgccgtggcattcggggcagccgccacgccgcggggaccggcgtcggcgtcggcggcgctctgcgcgagggtgcctgccgctgccggcggcatgcgtcgctccggggtggccgtgcggtgcgagaaatctagcatcgctgtcatcgggctgtctgtccacactgcccctgtcgagatgcgggagaaactagcggtggccgaggagctgtggccgagggccattagtgagttgacgtccttaaatcacatcgaagaagcagccgtgttatctacatgcaacagaatggaaatttatgttgttgcgctgtcgtggaaccggggcattcgcgaggtcgtggactggatgtccaagaaaagcggcatcccggcttctgagttgcgtgagcatctcttcatgctgcgcgacagtgatgccacaagacacttgtttgaagtatctgcaggcctcgattccctcgttttgggagagggacagatcctggctcaagtgaagcaggtggtcaggaatggccagaacagtggcggtttaggtaaaaatattgatcgaatgttcaaagatgctattactgctgggaagcgagttcgatgtgaaacaaatatctccgcgggcgcggtatccgtgtcctcagcggccgtggaacttgcaatgatgaaacttccaaaatcagagtgcctatcggcccgtatgctccttataggagccggaaaaatgggcaagctggtcgtcaagcatctgattgcaaaggggtgcaagaaggttgttgtggtcaaccggtcagtggagagggttgacgccatcagggaggaaatgaaagacattgaaatcgtgtacaggcctctcaccgagatgtacgaagctgcggccgacgctgacgtagtcttcacgtcaactgcatcagagtcgctgctttttaccaaggagcacgccgaggtgttgcccccgatatcgctcgcgatgggcggtgtccggctgttcgtcgacataagcgtgccgcggaatgtgggtgcttgtcttagcgaggttgagcacgcccgcgtgtacaatgttgatgatctgaaggaagtcgttgaagccaacaaggaagaccgtgtaaggaaggcaatggaggcgcagactatcatcacccaagaactcaagaggttcgaggcctggcgcgattctcttgaaactgtgccgaccattaagaagctgcgctcatatgctgacagaatacgtgcttcggaattggagaagtgcctccaaaagataggcgaagataacctcaacaagaagatgaggagaagtattgaggagctgagcaccgggattgtcaataaacttctacatggtccattgcagcacctccgctgtgacggctctgattccagaacactggacgagacacttgaaaacatgcacgcgctaaatcgcatgtttagcctcgatacggagaaagcggttcttgagcaaaaaataaaggcgaaggtagaaaagacccagagctgacctaggctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccag

SEQ ID NO: 70 - DNA, 2323 bp, synthetic construct
  source 1..2323: mol_type = other DNA
  misc_feature 1..2323: note = pAG5030
caccagagcggccctcctgagcacaagctgtttggattcacctaccttcggctgtcgaatcagctggtggagggacaaaactatgtcaatttcaagacctttgtcgacagaatgcatgccaacctggttagtgccacaaccacttactaacgcatgtcaaaaattaaacatatacaagaaccatttgttgatttgcaggtgcctattatatactaataatttaattttattgttttcagcctcgtgacccatatgttgatccaatggcgcctttgccaagatcagggccagaaatatcgattgagatgatcctacaagcagcacagccaaaactgcagccattccccttccaggagcacaccgacctgccagtaggccctactggtggcatgggtgggcaggctgaaggccccacctgtggcatgggtgggcaagttaaaggccctactggtggcatgggtgggcaggctgaagaccctactagtggcataggtggggagctccctgccaccatgtaatggaacctttatgatttactaccctttatgttatgtgtgagtgtgacagagaaacctttctctgccttattaataataaataaagcacatcacttgtgtgtgttctgaaaaaaagatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcaccgtacgacgacgacgaggggcggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 71 - DNA, 2452 bp, synthetic construct
  source 1..2452: mol_type = other DNA
  misc_feature 1..2452: note = pAG5031
caccagagcggccctcctgagcacaagctgtttggattcacctaccttcggctgtcgaatcagctggtggagggacaaaactatgtcaatttcaagacctttgtcgacagaatgcatgccaacctggttagtgccacaaccacttactaacgcatgtcaaaaattaaacatatacaagaaccatttgttgatttgcaggtgcctattatatactaataatttaattttattgttttcagcctcgtgacccatatgttgatccaatggcgcctttgccaagatcagggccagaaatatcgattgagatgatcctacaagcagcacagccaaaactgcagccattccccttccaggagcacaccgacctgccagtaggccctactggtggcatgggtgggcaggctgaaggccccacctgtggcatgggtgggcaagttaaaggccctactggtggcatgggtgggcaggctgaagaccctactagtggcataggtggggagctccctgccaccatgtaatggaacctttatgatttactaccctttatgttatgtgtgagtgtgacagagaaacctttctctgccttattaataataaataaagcacatcacttgtgtgtgttctgaaaaaaagatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcaccgtacgacgacgacgaggggcggatcctaaaccatgcgcgtgctgctcgtggccctggccctgcttgctcttgccgctagcgccacctctgcccaaacatgtctgaccagcccacaaacaggattccacaacggcttcttttactcgttttggaaagattctcctggtactgttaacttttgccttcttgaagggggaagatatacctccaattggtctgggataaacaattgggtcggtggcaagggctggcagaccggtagtaggcgcaacatcacgtattccgggagcttcaatacaccagggaatggatacctggctctctacggttggaccaccaacccgttggttgagtactatgttgtcgatagctggggctcctggcgccctcccggaagtgatgggacatttcttggcacagtgaactcagacggcggcacgtatgacatctacagggcgcaaagagtcaacgcaccgagcatcattggcaatgccaccttctaccagtattggtccgtgcggcagtccaagcgtgtcggtgggacaatcacgactggcaaccacttcgacgcgtgggccagcgtgggcctcaacctcgggactcataactaccagataatggctactgagggttaccagtcgtcaggatcttcagacattacggtgtcaagcgagaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 72 - DNA, 3375 bp, synthetic construct
  source 1..3375: mol_type = other DNA
  misc_feature 1..3375: note = pAG5032
aaatacatgcatggcgtcatcatggataaatctctaagccttctataattatatgagttagagcaacacatatctaactaggtacagaacatgcatggtcaattttgacacaaattgaaacacgtgatgtgatcattgaataagggggtgtttggttagagggactaaagattagtctctagtttttagtcccatttagtcccttttttgccaaacactaggactaaaaaatggactaaaataatttagtctttagtccttcacataggtgctaaaagagactaaaagctcataattatcatgttgcccttacctttttgtatggataactaattatgagtatattctaagggcatttgagtctttggacaatgtatttaatgactttagaatatgtttagtccctagaaccaaacatgtagggactaaagtttagtgctaggactaaagtttagtcctagcactaattgaaaccaaacatggcctaaacctcgaagctagccacatgttccacaagtgatcaggcaacaagttgtgtcccattgcaacgcatgagaaagtttgtgagctgttgcgtatatgtgcagtgcttgggtagaactctcatagtagagttagaggcctgtttggtagagctcaattatgtgatcctctagtgaagtaattttagtgtagaagcggagctgagagaaggtttgaattggtctaatgttgttccggtgaggtttagctcatttggtagagctctgctccatgattctctagctccgagagcgctgattctctaatagagtgattctgagtgattctctactggaagtgaatctattttgactaaaattgtttgtcaaataggttgtgaagtgattcctgagggttaaagaatagggagcaggttgggagtgacggaaagcaggttattttggttcccactttctagtacaaagtaaagactagattcactccactcccaacataaaacagtttagatttttatcgtttggctcgccaagagtgattctcgctgggagcgcacgaagctatgggagctctgccaaataccccaaattaaatgaagaagtgaacgaagaagtgattcttgtccatacctactagaatttgtatgagcagagctagagtaattatttactaaaaaatctgctccatatagagcgctaccaaatgaagagccgaataaatttgtacaaattctctcgttaaaccagagagaaaatgttatatatggtgactcaaaagtttgccacgttattttagattaacctaagttgttagatatgtatgtctgtccattcgattaggggtgctaaaaatattatcaaaactaattaactaaaaaaatttcatttaaaaagtaattgaacccaaaatatcatggtatgtttggtgaagacagtgatcagtgatttttttatatctatatatatatcaaagatacttgattttctagaaggttctttttgttgttttcccttatgtttttacgcatgatgcaattctttttgagaggtttccgatgcattgatgttattgtattatctcctatatataggtcgacgtacattatgtattgcaataaccagttaactcgatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcatgcaactctagtattgacacttgtatagcatataatgccaatacgacacctgccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactctaaatcactcacaacacaacacatatcttttagtaacctttcaataggcgtcccccaagaactagtaaacggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 73 - DNA, 3504 bp, synthetic construct
  source 1..3504: mol_type = other DNA
  misc_feature 1..3504: note = pAG5033
aaatacatgcatggcgtcatcatggataaatctctaagccttctataattatatgagttagagcaacacatatctaactaggtacagaacatgcatggtcaattttgacacaaattgaaacacgtgatgtgatcattgaataagggggtgtttggttagagggactaaagattagtctctagtttttagtcccatttagtcccttttttgccaaacactaggactaaaaaatggactaaaataatttagtctttagtccttcacataggtgctaaaagagactaaaagctcataattatcatgttgcccttacctttttgtatggataactaattatgagtatattctaagggcatttgagtctttggacaatgtatttaatgactttagaatatgtttagtccctagaaccaaacatgtagggactaaagtttagtgctaggactaaagtttagtcctagcactaattgaaaccaaacatggcctaaacctcgaagctagccacatgttccacaagtgatcaggcaacaagttgtgtcccattgcaacgcatgagaaagtttgtgagctgttgcgtatatgtgcagtgcttgggtagaactctcatagtagagttagaggcctgtttggtagagctcaattatgtgatcctctagtgaagtaattttagtgtagaagcggagctgagagaaggtttgaattggtctaatgttgttccggtgaggtttagctcatttggtagagctctgctccatgattctctagctccgagagcgctgattctctaatagagtgattctgagtgattctctactggaagtgaatctattttgactaaaattgtttgtcaaataggttgtgaagtgattcctgagggttaaagaatagggagcaggttgggagtgacggaaagcaggttattttggttcccactttctagtacaaagtaaagactagattcactccactcccaacataaaacagtttagatttttatcgtttggctcgccaagagtgattctcgctgggagcgcacgaagctatgggagctctgccaaataccccaaattaaatgaagaagtgaacgaagaagtgattcttgtccatacctactagaatttgtatgagcagagctagagtaattatttactaaaaaatctgctccatatagagcgctaccaaatgaagagccgaataaatttgtacaaattctctcgttaaaccagagagaaaatgttatatatggtgactcaaaagtttgccacgttattttagattaacctaagttgttagatatgtatgtctgtccattcgattaggggtgctaaaaatattatcaaaactaattaactaaaaaaatttcatttaaaaagtaattgaacccaaaatatcatggtatgtttggtgaagacagtgatcagtgatttttttatatctatatatatatcaaagatacttgattttctagaaggttctttttgttgttttcccttatgtttttacgcatgatgcaattctttttgagaggtttccgatgcattgatgttattgtattatctcctatatataggtcgacgtacattatgtattgcaataaccagttaactcgatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcatgcaactctagtattgacacttgtatagcatataatgccaatacgacacctgccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactctaaatcactcacaacacaacacatatcttttagtaacctttcaataggcgtcccccaagaactagtaaacggatcctaaaccatgcgcgtgctgctcgtggccctggccctgcttgctcttgccgctagcgccacctctgcccaaacatgtctgaccagcccacaaacaggattccacaacggcttcttttactcgttttggaaagattctcctggtactgttaacttttgccttcttgaagggggaagatatacctccaattggtctgggataaacaattgggtcggtggcaagggctggcagaccggtagtaggcgcaacatcacgtattccgggagcttcaatacaccagggaatggatacctggctctctacggttggaccaccaacccgttggttgagtactatgttgtcgatagctggggctcctggcgccctcccggaagtgatgggacatttcttggcacagtgaactcagacggcggcacgtatgacatctacagggcgcaaagagtcaacgcaccgagcatcattggcaatgccaccttctaccagtattggtccgtgcggcagtccaagcgtgtcggtgggacaatcacgactggcaaccacttcgacgcgtgggccagcgtgggcctcaacctcgggactcataactaccagataatggctactgagggttaccagtcgtcaggatcttcagacattacggtgtcaagcgagaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 74 - DNA, 1790 bp, synthetic construct
  source 1..1790: mol_type = other DNA
  misc_feature 1..1790: note = pAG5034
ggtaccgttttcaaatttgaattttaatgtgtgttgtaagtataaatttaaaataaaaataaaaacaattattatatcaaaatggcaaaaacatttaatacgtattatttattaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgttcattgcatttgactatgtggatagtgttttgatccatgcccttcatttgccgctattaattaatttggtaacagattcgttctaatcagttacttaatccttcctcatcataattaatctggtagttcgaatgccataatattgattagttttttggaccataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggcgtcaatcgagcagcccaaaacattcaccaactcaacccatcatgagcccacacatttgttgtttctaacccaacctcaaactcgtattctcttccgccacctcatttttgtttatttcaacacccgtcaaactgcatcccaccccgtggccaaatgttcatgcatgttaacaagacctatgactataaatatctgcaatctcggcccaagttttcatcatcaagaaccagttcaatatcctagtacgccgtattaaagaatttaagatatactggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 75 - DNA, 5704 bp, synthetic construct
  source 1..5704: mol_type = other DNA
  misc_feature 1..5704: note = pAG5035
caccagagcggccctcctgagcacaagctgtttggattcacctaccttcggctgtcgaatcagctggtggagggacaaaactatgtcaatttcaagacctttgtcgacagaatgcatgccaacctggttagtgccacaaccacttactaacgcatgtcaaaaattaaacatatacaagaaccatttgttgatttgcaggtgcctattatatactaataatttaattttattgttttcagcctcgtgacccatatgttgatccaatggcgcctttgccaagatcagggccagaaatatcgattgagatgatcctacaagcagcacagccaaaactgcagccattccccttccaggagcacaccgacctgccagtaggccctactggtggcatgggtgggcaggctgaaggccccacctgtggcatgggtgggcaagttaaaggccctactggtggcatgggtgggcaggctgaagaccctactagtggcataggtggggagctccctgccaccatgtaatggaacctttatgatttactaccctttatgttatgtgtgagtgtgacagagaaacctttctctgccttattaataataaataaagcacatcacttgtgtgtgttctgaaaaaaagatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcaccgtacgacgacgacgaggggcggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccaaatacatgcatggcgtcatcatggataaatctctaagccttctataattatatgagttagagcaacacatatctaactaggtacagaacatgcatggtcaattttgacacaaattgaaacacgtgatgtgatcattgaataagggggtgtttggttagagggactaaagattagtctctagtttttagtcccatttagtcccttttttgccaaacactaggactaaaaaatggactaaaataatttagtctttagtccttcacataggtgctaaaagagactaaaagctcataattatcatgttgcccttacctttttgtatggataactaattatgagtatattctaagggcatttgagtctttggacaatgtatttaatgactttagaatatgtttagtccctagaaccaaacatgtagggactaaagtttagtgctaggactaaagtttagtcctagcactaattgaaaccaaacatggcctaaacctcgaagctagccacatgttccacaagtgatcaggcaacaagttgtgtcccattgcaacgcatgagaaagtttgtgagctgttgcgtatatgtgcagtgcttgggtagaactctcatagtagagttagaggcctgtttggtagagctcaattatgtgatcctctagtgaagtaattttagtgtagaagcggagctgagagaaggtttgaattggtctaatgttgttccggtgaggtttagctcatttggtagagctctgctccatgattctctagctccgagagcgctgattctctaatagagtgattctgagtgattctctactggaagtgaatctattttgactaaaattgtttgtcaaataggttgtgaagtgattcctgagggttaaagaatagggagcaggttgggagtgacggaaagcaggttattttggttcccactttctagtacaaagtaaagactagattcactccactcccaacataaaacagtttagatttttatcgtttggctcgccaagagtgattctcgctgggagcgcacgaagctatgggagctctgccaaataccccaaattaaatgaagaagtgaacgaagaagtgattcttgtccatacctactagaatttgtatgagcagagctagagtaattatttactaaaaaatctgctccatatagagcgctaccaaatgaagagccgaataaatttgtacaaattctctcgttaaaccagagagaaaatgttatatatggtgactcaaaagtttgccacgttattttagattaacctaagttgttagatatgtatgtctgtccattcgattaggggtgctaaaaatattatcaaaactaattaactaaaaaaatttcatttaaaaagtaattgaacccaaaatatcatggtatgtttggtgaagacagtgatcagtgatttttttatatctatatatatatcaaagatacttgattttctagaaggttctttttgttgttttcccttatgtttttacgcatgatgcaattctttttgagaggtttccgatgcattgatgttattgtattatctcctatatataggtcgacgtacattatgtattgcaataaccagttaactcgatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcatgcaactctagtattgacacttgtatagcatataatgccaatacgacacctgccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactctaaatcactcacaacacaacacatatcttttagtaacctttcaataggcgtcccccaagaactagtaaacggatccaccatgagggtgttgctcgttgccctcgctctcctggctctcgctgcgagcgccaccagcgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggaaaggacgagctgtgacctaggtccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattg

SEQ ID NO: 76 - DNA, 4033 bp, synthetic construct
  source 1..4033: mol_type = other DNA
  misc_feature 1..4033: note = pAG5036
aaatacatgcatggcgtcatcatggataaatctctaagccttctataattatatgagttagagcaacacatatctaactaggtacagaacatgcatggtcaattttgacacaaattgaaacacgtgatgtgatcattgaataagggggtgtttggttagagggactaaagattagtctctagtttttagtcccatttagtcccttttttgccaaacactaggactaaaaaatggactaaaataatttagtctttagtccttcacataggtgctaaaagagactaaaagctcataattatcatgttgcccttacctttttgtatggataactaattatgagtatattctaagggcatttgagtctttggacaatgtatttaatgactttagaatatgtttagtccctagaaccaaacatgtagggactaaagtttagtgctaggactaaagtttagtcctagcactaattgaaaccaaacatggcctaaacctcgaagctagccacatgttccacaagtgatcaggcaacaagttgtgtcccattgcaacgcatgagaaagtttgtgagctgttgcgtatatgtgcagtgcttgggtagaactctcatagtagagttagaggcctgtttggtagagctcaattatgtgatcctctagtgaagtaattttagtgtagaagcggagctgagagaaggtttgaattggtctaatgttgttccggtgaggtttagctcatttggtagagctctgctccatgattctctagctccgagagcgctgattctctaatagagtgattctgagtgattctctactggaagtgaatctattttgactaaaattgtttgtcaaataggttgtgaagtgattcctgagggttaaagaatagggagcaggttgggagtgacggaaagcaggttattttggttcccactttctagtacaaagtaaagactagattcactccactcccaacataaaacagtttagatttttatcgtttggctcgccaagagtgattctcgctgggagcgcacgaagctatgggagctctgccaaataccccaaattaaatgaagaagtgaacgaagaagtgattcttgtccatacctactagaatttgtatgagcagagctagagtaattatttactaaaaaatctgctccatatagagcgctaccaaatgaagagccgaataaatttgtacaaattctctcgttaaaccagagagaaaatgttatatatggtgactcaaaagtttgccacgttattttagattaacctaagttgttagatatgtatgtctgtccattcgattaggggtgctaaaaatattatcaaaactaattaactaaaaaaatttcatttaaaaagtaattgaacccaaaatatcatggtatgtttggtgaagacagtgatcagtgatttttttatatctatatatatatcaaagatacttgattttctagaaggttctttttgttgttttcccttatgtttttacgcatgatgcaattctttttgagaggtttccgatgcattgatgttattgtattatctcctatatataggtcgacgtacattatgtattgcaataaccagttaactcgatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcatgcaactctagtattgacacttgtatagcatataatgccaatacgacacctgccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactctaaatcactcacaacacaacacatatcttttagtaacctttcaataggcgtcccccaagaactagtaaacggatccaccatggggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggatgacctaggtgatatcgcaagagggaagcgcttgacgtagtctttagatttcaattgttgcagccgatcaaccttcttagggttgtataatatgaagtcaatgttgagactaatgtgaggtgggagaggcctattgatgcatcaaaagtattatgagctaataataaagccttctccacagattttatgattgctcaagaactgtgaggtgctaatgtgctatgcgacaatgttttttttcctgcctagcagcttctcatacaaagaggcccacagtcttctcatacaaagcggcccacagtgttcggcttgccaggccagcctggataggagtccagcccatgatagcgggcctaccacaccaaaacgcgccgcatattaagttgccctcgctggcaccgtggcacggccactcacgtaccggcaggccattggccacagcccgctttactcacgacgcgacgaaccgcgcactccttttaaagctccacgttgcggtgtcctcacgggtcacggcgaccgatgccgctgctgctgcgcggcgcctccctcctccgcctctgccagtacggtgcctatctccccccaaaccctcccttgctctctcatctcatatggtgcatttttttaaccttcgactcggttcagcccatctcgtctgcgtcgactcgattcttcgccattcaacgggcaccctcaatagggggacgagtccatcgattgtttggcggcgagttaaaatatccgcttcttttattgtgcgctttcaactgtcaacggagtcgtcgatgtggattatctaatttagtaaaaataatatgctagggagcataatgtcacaaacgcgggtgcacttgaggctttttctttgaggaatcttgttacttcagctcaaactcatttgcacataatgtgaaacctaaatcctatgtcaacctagaaaaaaaatccatcagtgcaagaggatgtttgtccacagttatctcatacctggattcgttgtcacaag

SEQ ID NO: 77 - DNA, 4087 bp, synthetic construct
  source 1..4087: mol_type = other DNA
  misc_feature 1..4087: note = pAG5037
aaatacatgcatggcgtcatcatggataaatctctaagccttctataattatatgagttagagcaacacatatctaactaggtacagaacatgcatggtcaattttgacacaaattgaaacacgtgatgtgatcattgaataagggggtgtttggttagagggactaaagattagtctctagtttttagtcccatttagtcccttttttgccaaacactaggactaaaaaatggactaaaataatttagtctttagtccttcacataggtgctaaaagagactaaaagctcataattatcatgttgcccttacctttttgtatggataactaattatgagtatattctaagggcatttgagtctttggacaatgtatttaatgactttagaatatgtttagtccctagaaccaaacatgtagggactaaagtttagtgctaggactaaagtttagtcctagcactaattgaaaccaaacatggcctaaacctcgaagctagccacatgttccacaagtgatcaggcaacaagttgtgtcccattgcaacgcatgagaaagtttgtgagctgttgcgtatatgtgcagtgcttgggtagaactctcatagtagagttagaggcctgtttggtagagctcaattatgtgatcctctagtgaagtaattttagtgtagaagcggagctgagagaaggtttgaattggtctaatgttgttccggtgaggtttagctcatttggtagagctctgctccatgattctctagctccgagagcgctgattctctaatagagtgattctgagtgattctctactggaagtgaatctattttgactaaaattgtttgtcaaataggttgtgaagtgattcctgagggttaaagaatagggagcaggttgggagtgacggaaagcaggttattttggttcccactttctagtacaaagtaaagactagattcactccactcccaacataaaacagtttagatttttatcgtttggctcgccaagagtgattctcgctgggagcgcacgaagctatgggagctctgccaaataccccaaattaaatgaagaagtgaacgaagaagtgattcttgtccatacctactagaatttgtatgagcagagctagagtaattatttactaaaaaatctgctccatatagagcgctaccaaatgaagagccgaataaatttgtacaaattctctcgttaaaccagagagaaaatgttatatatggtgactcaaaagtttgccacgttattttagattaacctaagttgttagatatgtatgtctgtccattcgattaggggtgctaaaaatattatcaaaactaattaactaaaaaaatttcatttaaaaagtaattgaacccaaaatatcatggtatgtttggtgaagacagtgatcagtgatttttttatatctatatatatatcaaagatacttgattttctagaaggttctttttgttgttttcccttatgtttttacgcatgatgcaattctttttgagaggtttccgatgcattgatgttattgtattatctcctatatataggtcgacgtacattatgtattgcaataaccagttaactcgatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcatgcaactctagtattgacacttgtatagcatataatgccaatacgacacctgccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactctaaatcactcacaacacaacacatatcttttagtaacctttcaataggcgtcccccaagaactagtaaacggatccaccatggcaaaactgttgagcctgttcttagccctctcctttgtagcagctatgtttgccgggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttcatcagcgacgccatcattcacgtcctgcacgccaagcacccttcagacttcggcgctgacgctcaagcggccatgtccaaagcgctggagctgttccgcaatgatatggccgcgcagtacaaggttcttgggtttcatggatgacctaggtgatatcgcaagagggaagcgcttgacgtagtctttagatttcaattgttgcagccgatcaaccttcttagggttgtataatatgaagtcaatgttgagactaatgtgaggtgggagaggcctattgatgcatcaaaagtattatgagctaataataaagccttctccacagattttatgattgctcaagaactgtgaggtgctaatgtgctatgcgacaatgttttttttcctgcctagcagcttctcatacaaagaggcccacagtcttctcatacaaagcggcccacagtgttcggcttgccaggccagcctggataggagtccagcccatgatagcgggcctaccacaccaaaacgcgccgcatattaagttgccctcgctggcaccgtggcacggccactcacgtaccggcaggccattggccacagcccgctttactcacgacgcgacgaaccgcgcactccttttaaagctccacgttgcggtgtcctcacgggtcacggcgaccgatgccgctgctgctgcgcggcgcctccctcctccgcctctgccagtacggtgcctatctccccccaaaccctcccttgctctctcatctcatatggtgcatttttttaaccttcgactcggttcagcccatctcgtctgcgtcgactcgattcttcgccattcaacgggcaccctcaatagggggacgagtccatcgattgtttggcggcgagttaaaatatccgcttcttttattgtgcgctttcaactgtcaacggagtcgtcgatgtggattatctaatttagtaaaaataatatgctagggagcataatgtcacaaacgcgggtgcacttgaggctttttctttgaggaatcttgttacttcagctcaaactcatttgcacataatgtgaaacctaaatcctatgtcaacctagaaaaaaaatccatcagtgcaagaggatgtttgtccacagttatctcatacctggattcgttgtcacaag

SEQ ID NO: 78 - DNA, 459 bp, Bos taurus
  source 1..459: mol_type = other DNA
  misc_feature 1..459: note = BtMYGm coding sequence
gggttaagtgatggagaatggcagctggtgctcaatgcatggggcaaggtggaggcagatgtggcgggtcacggccaagaagttctcatcaggctctttaccggtcatccagagacgctggagaagtttgacaagttcaagcatctgaaaacagaagcagagatgaaggccagcgaggatctgaagaaacacggtaacactgtccttacagctctgggcggcattttgaagaagaagggacaccatgaagccgaggtcaaacatttggcagaatctcacgccaacaagcacaagataccggtaaaatatctagagttccacagcgacgccatcattcacgtcctgcacgccaagcacccttcagacttcggcgctgacgctcaagcggccatgtccaaagcgctggagctgttccgcaatgatatggccgcgcagtacaaggttcttgggtttcatgga

SEQ ID NO: 79 - DNA, 1365 bp, Hordeum vulgare
  source 1..1365: mol_type = other DNA
  misc_feature 1..1365: note = Barley glutamyl-tRNA reductase HvGluTRm_truncated coding sequence
gagaaatctagcatcgctgtcatcgggctgtctgtccacactgcccctgtcgagatgcgggagaaactagcggtggccgaggagctgtggccgagggccattagtgagttgacgtccttaaatcacatcgaagaagcagccgtgttatctacatgcaacagaatggaaatttatgttgttgcgctgtcgtggaaccggggcattcgcgaggtcgtggactggatgtccaagaaaagcggcatcccggcttctgagttgcgtgagcatctcttcatgctgcgcgacagtgatgccacaagacacttgtttgaagtatctgcaggcctcgattccctcgttttgggagagggacagatcctggctcaagtgaagcaggtggtcaggaatggccagaacagtggcggtttaggtaaaaatattgatcgaatgttcaaagatgctattactgctgggaagcgagttcgatgtgaaacaaatatctccgcgggcgcggtatccgtgtcctcagcggccgtggaacttgcaatgatgaaacttccaaaatcagagtgcctatcggcccgtatgctccttataggagccggaaaaatgggcaagctggtcgtcaagcatctgattgcaaaggggtgcaagaaggttgttgtggtcaaccggtcagtggagagggttgacgccatcagggaggaaatgaaagacattgaaatcgtgtacaggcctctcaccgagatgtacgaagctgcggccgacgctgacgtagtcttcacgtcaactgcatcagagtcgctgctttttaccaaggagcacgccgaggtgttgcccccgatatcgctcgcgatgggcggtgtccggctgttcgtcgacataagcgtgccgcggaatgtgggtgcttgtcttagcgaggttgagcacgcccgcgtgtacaatgttgatgatctgaaggaagtcgttgaagccaacaaggaagaccgtgtaaggaaggcaatggaggcgcagactatcatcacccaagaactcaagaggttcgaggcctggcgcgattctcttgaaactgtgccgaccattaagaagctgcgctcatatgctgacagaatacgtgcttcggaattggagaagtgcctccaaaagataggcgaagataacctcaacaagaagatgaggagaagtattgaggagctgagcaccgggattgtcaataaacttctacatggtccattgcagcacctccgctgtgacggctctgattccagaacactggacgagacacttgaaaacatgcacgcgctaaatcgcatgtttagcctcgatacggagaaagcggttcttgagcaaaaaataaaggcgaaggtagaaaagacccagagctga

SEQ ID NO: 80 - DNA, 153 bp, Zea mays
  source 1..153: mol_type = other DNA
  misc_feature 1..153: note = Maize glutamyl tRNA reductase signal sequence ZmGluRTss coding sequence
atgatggcgagcacggctccagcggcctccaccgccgtggcattcggggcagccgccacgccgcggggaccggcgtcggcgtcggcggcgctctgcgcgagggtgcctgccgctgccggcggcatgcgtcgctccggggtggccgtgcggtgc

SEQ ID NO: 81 - DNA, 1455 bp, Zea mays
  source 1..1455: mol_type = other DNA
  misc_feature 1..1455: note = Maize ferrochelatase ZmFC1A coding sequence
atgtcttcgtcgggcccctccccggcgacgggaatccacgcgtcgccgccgttgggccttttgccggcgacgggaacccatcacaccaggtcatggggcaaaacaacctccacaagttttactggttctaccaccaaacatgagcagagcttgcatggaaatgttaagccgttgcaattggcggcaaatgaatcctctcgtttggcttacagaagtccagcacttaaaaaccagtggaatcttcctgctagttcttcctccactaatgtggttaccacctttgatgataacgaacacgtgtcttccagtgttattgaagaaaaagttggagtactgttattaaaccttggtggtccagagacacttgacgatgttcaaccatttttattcaacctatttgctgatccagatatcattcgactccctagactcttcaggtttcttcaaagaccactggccaaacttatttctacttttagagctcctaagagtaaagaagggtatgcttcaattggtggtgggtcgccgttaaggaaaattactgatgaacaggcgaatgctttgaagattgccctggaaaagaaaaaattgaacgcaaacatatatgttgggatgcggtattggtaccctttcacagaagaggccattgatcaaattaaaaaggataagattaccaagctcgttgttcttcccctttaccctcagtactccatatcaacaagtgggtcaagcattcgtgttctccaagacattgtcaaggaagattcatatttttctggtttgccaatttccattattgaatcatggtaccaacgagatggctatgtgaaatcaatgtctgacctaattgaaaaggagctctccgccttctccaatcctgaagaggttatgatattcttcagtgcacatggtgtgccacttacctatgttgaggatgctggagatccttacagagatcagatggaggattgtattgctttgatcatgggggagttaagatcaagaggaatcttaaatagtcacactttggcgtaccagagtcgggtggggccagttcaatggctgaagccatatactgatgaagttttagtagaacttggtcaaaagggtgtgaagagcctcctggctgttccagtaagctttgtgagtgagcacatcgagacattggaagaaattgacatggagtacaaggagttggctctggaatcaggcatcaagaactggggtcgggttcctgctcttggatgcacttcaacattcatctccgaccttgcagatgctgtcgtcgaagccctgccctctgcttcagcgctcgcgaccagaaagcctgacgacatcgattccagcatggacctgacgcattacctcaccaagatgctgtttggctcaatcttggcatttgtcctgctgctatcaccaaggctagtttccgccttccggagcaccatgctttaa

SEQ ID NO: 82 - DNA, 57 bp, Zea mays
  source 1..57: mol_type = other DNA
  misc_feature 1..57: note = Signal sequence of the maize Al9 coding sequence
atggcaaaactgttgagcctgttcttagccctctcctttgtagcagctatgtttgcc

SEQ ID NO: 83 - DNA, 1502 bp, Hordeum vulgare
  source 1..1502: mol_type = other DNA
  misc_feature 1..1502: note = prHvLtp2promoter
caccagagcggccctcctgagcacaagctgtttggattcacctaccttcggctgtcgaatcagctggtggagggacaaaactatgtcaatttcaagacctttgtcgacagaatgcatgccaacctggttagtgccacaaccacttactaacgcatgtcaaaaattaaacatatacaagaaccatttgttgatttgcaggtgcctattatatactaataatttaattttattgttttcagcctcgtgacccatatgttgatccaatggcgcctttgccaagatcagggccagaaatatcgattgagatgatcctacaagcagcacagccaaaactgcagccattccccttccaggagcacaccgacctgccagtaggccctactggtggcatgggtgggcaggctgaaggccccacctgtggcatgggtgggcaagttaaaggccctactggtggcatgggtgggcaggctgaagaccctactagtggcataggtggggagctccctgccaccatgtaatggaacctttatgatttactaccctttatgttatgtgtgagtgtgacagagaaacctttctctgccttattaataataaataaagcacatcacttgtgtgtgttctgaaaaaaagatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcaccgtacgacgacgacgaggggcg

SEQ ID NO: 84 - DNA, 2553 bp, Zea mays
  source 1..2553: mol_type = other DNA
  misc_feature 1..2553: note = prZmAl9 promoter
aaatacatgcatggcgtcatcatggataaatctctaagccttctataattatatgagttagagcaacacatatctaactaggtacagaacatgcatggtcaattttgacacaaattgaaacacgtgatgtgatcattgaataagggggtgtttggttagagggactaaagattagtctctagtttttagtcccatttagtcccttttttgccaaacactaggactaaaaaatggactaaaataatttagtctttagtccttcacataggtgctaaaagagactaaaagctcataattatcatgttgcccttacctttttgtatggataactaattatgagtatattctaagggcatttgagtctttggacaatgtatttaatgactttagaatatgtttagtccctagaaccaaacatgtagggactaaagtttagtgctaggactaaagtttagtcctagcactaattgaaaccaaacatggcctaaacctcgaagctagccacatgttccacaagtgatcaggcaacaagttgtgtcccattgcaacgcatgagaaagtttgtgagctgttgcgtatatgtgcagtgcttgggtagaactctcatagtagagttagaggcctgtttggtagagctcaattatgtgatcctctagtgaagtaattttagtgtagaagcggagctgagagaaggtttgaattggtctaatgttgttccggtgaggtttagctcatttggtagagctctgctccatgattctctagctccgagagcgctgattctctaatagagtgattctgagtgattctctactggaagtgaatctattttgactaaaattgtttgtcaaataggttgtgaagtgattcctgagggttaaagaatagggagcaggttgggagtgacggaaagcaggttattttggttcccactttctagtacaaagtaaagactagattcactccactcccaacataaaacagtttagatttttatcgtttggctcgccaagagtgattctcgctgggagcgcacgaagctatgggagctctgccaaataccccaaattaaatgaagaagtgaacgaagaagtgattcttgtccatacctactagaatttgtatgagcagagctagagtaattatttactaaaaaatctgctccatatagagcgctaccaaatgaagagccgaataaatttgtacaaattctctcgttaaaccagagagaaaatgttatatatggtgactcaaaagtttgccacgttattttagattaacctaagttgttagatatgtatgtctgtccattcgattaggggtgctaaaaatattatcaaaactaattaactaaaaaaatttcatttaaaaagtaattgaacccaaaatatcatggtatgtttggtgaagacagtgatcagtgatttttttatatctatatatatatcaaagatacttgattttctagaaggttctttttgttgttttcccttatgtttttacgcatgatgcaattctttttgagaggtttccgatgcattgatgttattgtattatctcctatatataggtcgacgtacattatgtattgcaataaccagttaactcgatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcatgcaactctagtattgacacttgtatagcatataatgccaatacgacacctgccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactctaaatcactcacaacacaacacatatcttttagtaacctttcaataggcgtcccccaagaactagtaaac

SEQ ID NO: 85 - DNA, 962 bp, Glycine max
  source 1..962: mol_type = other DNA
  misc_feature 1..962: note = prGmCG1 promoter
gttttcaaatttgaattttaatgtgtgttgtaagtataaatttaaaataaaaataaaaacaattattatatcaaaatggcaaaaacatttaatacgtattatttattaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgttcattgcatttgactatgtggatagtgttttgatccatgcccttcatttgccgctattaattaatttggtaacagattcgttctaatcagttacttaatccttcctcatcataattaatctggtagttcgaatgccataatattgattagttttttggaccataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggcgtcaatcgagcagcccaaaacattcaccaactcaacccatcatgagcccacacatttgttgtttctaacccaacctcaaactcgtattctcttccgccacctcatttttgtttatttcaacacccgtcaaactgcatcccaccccgtggccaaatgttcatgcatgttaacaagacctatgactataaatatctgcaatctcggcccaagttttcatcatcaagaaccagttcaatatcctagtacgccgtattaaagaatttaagatatact

SEQ ID NO: 86 - DNA, 1012 bp, Zea mays
  source 1..1012: mol_type = other DNA
  misc_feature 1..1012: note = ZmAl9T terminator
cctaggtgatatcgcaagagggaagcgcttgacgtagtctttagatttcaattgttgcagccgatcaaccttcttagggttgtataatatgaagtcaatgttgagactaatgtgaggtgggagaggcctattgatgcatcaaaagtattatgagctaataataaagccttctccacagattttatgattgctcaagaactgtgaggtgctaatgtgctatgcgacaatgttttttttcctgcctagcagcttctcatacaaagaggcccacagtcttctcatacaaagcggcccacagtgttcggcttgccaggccagcctggataggagtccagcccatgatagcgggcctaccacaccaaaacgcgccgcatattaagttgccctcgctggcaccgtggcacggccactcacgtaccggcaggccattggccacagcccgctttactcacgacgcgacgaaccgcgcactccttttaaagctccacgttgcggtgtcctcacgggtcacggcgaccgatgccgctgctgctgcgcggcgcctccctcctccgcctctgccagtacggtgcctatctccccccaaaccctcccttgctctctcatctcatatggtgcatttttttaaccttcgactcggttcagcccatctcgtctgcgtcgactcgattcttcgccattcaacgggcaccctcaatagggggacgagtccatcgattgtttggcggcgagttaaaatatccgcttcttttattgtgcgctttcaactgtcaacggagtcgtcgatgtggattatctaatttagtaaaaataatatgctagggagcataatgtcacaaacgcgggtgcacttgaggctttttctttgaggaatcttgttacttcagctcaaactcatttgcacataatgtgaaacctaaatcctatgtcaacctagaaaaaaaatccatcagtgcaagaggatgtttgtccacagttatctcatacctggattcgttgtcacaaggaattc

SEQ ID NO: 87 - DNA, 2071 bp, Oryza sativa
  source 1..2071: mol_type = other DNA
  misc_feature 1..2071: note = glutelin 1 gene (prGTL-03) promoter
tccatgctgtcctactacttgcttcatccccttctacattttgttctggtttttggcctgcatttcggatcatgatgtatgtgatttccaatctgctgcaatatgaatggagactctgtgctaaccatcaacaacatgaaatgcttatgaggcctttgctgagcagccaatcttgcctgtgtttatgtcttcacaggccgaattcctctgttttgtttttcaccctcaatatttggaaacatttatctaggttgtttgtgtccaggcctataaatcatacatgatgttgtcgtattggatgtgaatgtggtggcgtgttcagtgccttggatttgagtttgatgagagttgcttctgggtcaccactcaccattatcgatgctcctcttcagcataaggtaaaagtcttccctgtttacgttattttacccactatggttgcttgggttggttttttcctgattgcttatgccatggaaagtcatttgatatgttgaacttgaattaactgtagaattgtatacatgttccatttgtgttgtacttccttcttttctattagtagcctcagatgagtgtgaaaaaaacagattatataacttgccctataaatcatttgaaaaaaatattgtacagtgagaaattgatatatagtgaatttttaagagcatgttttcctaaagaagtatatattttctatgtacaaaggccattgaagtaattgtagatacaggataatgtagactttttggacttacactgctacctttaagtaacaatcatgagcaatagtgttgcaatgatatttaggctgcattcgtttactctcttgatttccatgagcacgcttcccaaactgttaaactctgtgttttttgccaaaaaaaaatgcataggaaagttgcttttaaaaaatcatatcaatccattttttaagttatagctaatacttaattaatcatgcgctaataagtcactctgtttttcgtactagagagattgttttgaaccagcactcaagaacacagccttaacccagccaaataatgctacaacctaccagtccacacctcttgtaaagcatttgttgcatggaaaagctaagatgacagcaacctgttcaggaaaacaactgacaaggtcatagggagagggagcttttggaaaggtgccgtgcagttcaaacaattagttagcagtagggtgttggtttttgctcacagcaataagaagttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgggcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaac

SEQ ID NO: 88 - DNA, 1474 bp, Oryza sativa
  source 1..1474: mol_type = other DNA
  misc_feature 1..1474: note = OsGluB4P promoter
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaagtttccaataacatcctcaaatagct

(19) United States
(12) Patent Application Publication
(10) Pub. No.: US 2025/0304987 A1
(43) Pub. Date: Oct. 2, 2025

(54) PLANTS EXPRESSING PROTEINS OF ANIMAL ORIGIN AND ASSOCIATED PROCESSES AND METHODS

(71) Applicant: AGRIVIDA, INC., Woburn, MS (US)

(72) Inventors: R. Michael RAAB, Arlington, MA (US); Oleg BOUGRI, Winchester, MA (US); Philip A. LESSARD, Framingham, MA (US)

(73) Assignee: AGRIVIDA, INC., Woburn, MA (US)

(21) Appl. No.: 18/864,257

(22) PCT Filed: May 12, 2023
(86) PCT No.: PCT/US2023/022026

§ 371 Date: Nov. 8, 2024

Related U.S. Application Data
(60) Provisional application No. 63/341,564, filed on May 13, 2022.
(60) Provisional application No. 63/403,989, filed on Sep. 6, 2022.

(51) Int. Cl. C12N 15/82; A23C 20/00; A23J 3/22; C07K 14/47; C07K 14/805; C12N 9/64

(52) U.S. Cl. CPC C12N 15/8257; A23C 20/00; A23J 3/227; C07K 14/4732; C07K 14/805; C12N 9/6483; C12N 15/8205; C12Y 304/23004

(57) ABSTRACT

The transgenic plants engineered to express animal-derived proteins, such as myoglobin, casein, chymosin, hemoglobin and actin and other animal-derived proteins, and the nucleic acids encoding the same are described herein. Methods of making and identifying the transgenic plants, processing the seeds from the transgenic plants that contain the expressed animal-derived proteins, isolating and purifying the expressed proteins, and utilizing the expressed proteins in food are provided. Food and food ingredients that include myoglobin, casein, chymosin, hemoglobin and actin are also disclosed herein. Genes encoding myoglobin, casein, hemoglobin and actin forms that have been modified to improve performance as components of food are described.

### CROSS REFERENCE TO RELATED APPLICATIONS

This application claims the benefit of U.S. provisional application No. 63/341,564, filed May 13, 2022, and U.S. provisional application No. 63/403,989, filed Sep. 6, 2022, both of which are incorporated by reference as if fully set forth.

The sequence listing electronically filed with this application titled “Sequence Listing XML,” which was created on May 12, 2023 and had a size of 232,579 bytes is incorporated by reference herein as if fully set forth.

### FIELD OF INVENTION

The disclosure relates to transgenic plants engineered to express myoglobin, casein, chymosin, and other animal-derived proteins, the nucleic acids encoding the same, as well as methods of making and identifying the transgenic plants, processing the seeds from the transgenic plants that contain the expressed proteins, isolating and purifying the expressed proteins, and utilizing the expressed proteins in food. The disclosure also relates to food and food ingredients that include myoglobin, casein, chymosin, hemoglobin and actin. The disclosure relates to genes encoding myoglobin, casein, hemoglobin and actin forms that have been modified to improve performance as components of food.

### BACKGROUND

Animal agriculture as a source of protein for human consumption is a significant contributor to global warming and is associated with a host of environmental problems. Despite the variation in these estimates, collectively these measurements consistently demonstrate the toll that animal agriculture takes on the environment and global climate.

To address these challenges, animal protein production by non-animal expression hosts has been proposed. While cellular protein production using microorganisms, animal cells, insect cells, or other isolated cells in bioreactors may address the environmental concerns, and has the added benefit of reducing the number of animals produced purely for human food, the economic challenges of producing animal proteins such as myoglobin, casein, and chymosin at costs competitive with animal agriculture are daunting. As an example, today bulk casein prices range between $8/lb to over $15/lb, however, the costs of most recombinant proteins are in the $100's/lb range, far above the prices of animal protein isolates made from animal agriculture.

In contrast to protein production via fermentation or cell culture, plant protein products are significantly less expensive. Protein products such as corn gluten meal and distillers dried grains and solubles (DDGS) sell for $0.40/lb-$1/lb, significantly less than recombinant protein production costs from fermentation or cell culture processes, and potentially less than the costs required to make these proteins through animal agriculture, which itself largely relies on plants as feed sources. Furthermore, recombinant protein production by plant hosts costs marginally more than producing the plant itself and an entire industry already exists for processing plant materials, particularly grain. These industrial processes fractionate plant tissues into their components parts so that they can be used in a variety of products including animal feed and feed ingredients, fermentation substrates, sugar and sweeteners, oils, fibers, and human food and food ingredients. Thus engineering plants to make animal derived proteins such as myoglobin, casein, and chymosin creates value by diverting protein production in the plant away from the production of proteins that have relatively low value (approximately $0.40/lb-$1/lb in 2021) to proteins that have higher value (over $8/lb and some near $100/lb in 2021).

Producing myoglobin (which is used in plant based meats), casein molecules (used in plant based dairy products, including cheese), and chymosin (also used in plant based dairy products including cheese) in plants is a way to cost effectively product such animal derived proteins without the detrimental environmental effects and greenhouse gas emissions that result from their production using animal agriculture. Further, by producing such molecules in corn grain, isolation of the desired protein can utilize existing corn processing facilities and technologies. Corn wet mills in particular are made to fractionate corn into starch, fiber, oil, and protein fractions. Similarly dry mills, and dry grind ethanol facilities, fractionate corn minimally into starch, fiber and protein streams, with some facilities having the ability to further degerminate the grain, separate oil and fractionate the fiber and protein streams. Using such processes to isolate the recombinantly produced myoglobin, casein, or chymosin enables separation of a valuable protein while still processing the other grain components and capturing their value as corn grits, corn gluten meal, corn gluten feed, corn germ meal, gluten, starch, sugar, dextrins, sweeteners, oil, fiber, bulk protein, food ingredients, and feed ingredients. Employing a mixed process wherein all fractions of the corn kernel can be utilized, including isolation of the recombinant protein, provides the best economics possible when using corn as a production host for myoglobin, actin, casein, and chymosin.

Isolation of the recombinant protein from these processes requires some novel steps to the existing process to adequately make use of the transgenic plant material and optimize recovery of the desired protein. Furthermore, protein properties and expression properties will impact the selection of optimal processing conditions to maximize the yield and purity of the recombinant protein. For example, there are multiple steps at which the recombinant protein can be isolated in the wet-milling processing including: from the steep water during the steeping step, following the hydrocyclone step to remove the germ, as part of the fiber wash when the corn gluten feed is isolated, during the starch and gluten separation or as recovery from the corn gluten meal, or prior to the sweetener refining or fermentation processes. In contrast to corn wet milling, there are fewer opportunities for protein extraction in the corn dry milling or corn dry grind processes. In dry milling, the purpose of which is to separate the germ and endosperm to make grits, flour, feed ingredients, and in some cases oil, there are fewer opportunities to do an aqueous extraction, in part because much of the process is designed to be run dry at low moisture levels. For dry milling, embryo expression of the recombinant protein may be preferred so that extraction and concentration can be performed on the separated germ, leaving the endosperm unmodified for continued processing. In the corn dry grind process, which produces ethanol, distillers dried grains and solubles, and oil, the optimal step for isolating the recombinant protein would be out of the slurry tank, prior to jet cooking and fermentation; in some cases if the protein is stable enough, it may be possible to isolate it after jet cooking, fermentation, or from the beer column. Alternatively, in dry grind processes that include a degermination step, the recombinant protein could be extracted directly from the endosperm following degermination, which would be especially advantageous for proteins expressed in the endosperm tissue or by aleurone cells. The optimal step, or steps, will depend on the protein properties, accumulation levels, tissue distribution of the recombinant protein in the grain, and value of the protein as higher value proteins will justify greater investment in their recovery.

Both myoglobin and chymosin are water soluble proteins that can be preferentially extracted using a water or buffer solvent, which has the benefit of removing aqueous soluble proteins while leaving much of the insoluble, zein and alcohol soluble, proteins behind. In contrast, casein molecules are hydrophobic and form micellular structures in water and aqueous environments, and their separation from an aqueous stream may be performed by decanting, centrifugation, filtration or other unit operations, or may be conducted using an organic solvent. In evaluating the isolation of the recombinant protein from the grain, we considered yield, purity, concentration, and impact on subsequent processing as parts of a technoeconomic model to help optimize the cost efficiency of removing the protein. Furthermore, we developed processes for both embryo and endosperm expression of myoglobin, chymosin, and various casein molecules. These processes involved novel modifications to the wet-milling, dry-milling, and dry-grind ethanol processes, which enabled isolation of the recombinant myoglobin, chymosin, and casein from the transgenic corn that expressed these recombinant proteins.

### SUMMARY

In an aspect, the invention relates to a transgenic plant or tissue thereof comprising a synthetic polynucleotide encoding at least one animal-derived protein, The animal-derived protein is selected from the group consisting of a myoglobin, hemoglobin, actin, chymosin, and casein proteins or any corresponding protein with sequence mutations.

In an aspect, the invention relates to an expression cassette comprising a synthetic nucleic acid encoding at least one animal-derived protein selected from the group consisting of a myoglobin, actin, hemoglobin, chymosin, and casein proteins.

In an aspect, the invention relates to a plant-based meat composition comprising at least one of the transgenic plants or tissues thereof expressing any one of the animal-derived proteins disclosed herein.

In an aspect, the invention relates to a plant-based meat composition comprising any one of the animal-derived proteins isolated from transgenic plants or tissues thereof disclosed herein.

In an aspect, the invention relates to a plant-based cheese composition comprising at least one of the transgenic plants or tissues thereof expressing any one of the animal-derived proteins disclosed herein.

In an aspect, the invention relates to a plant-based cheese composition comprising any one of the animal-derived proteins isolated from transgenic plants or tissues thereof disclosed herein.

In an aspect, the invention relates to a process for isolating at least one animal-derived protein from any one of transgenic plant or tissues thereof disclosed herein.

### BRIEF DESCRIPTION OF THE DRAWINGS

The following detailed description of preferred embodiments of the present invention will be better understood when read in conjunction with the appended drawings. For the purpose of illustrating the invention, particular embodiments are shown in the drawings. It is understood, however, that the invention is not limited to the precise arrangements and instrumentalities shown. In the drawings:

FIGS. 1A-1C illustrate tandem expression cassettes for myoglobin, hemoglobin, and actin in vectors pA5016 (FIG. 1A), pAG5017 (FIG. 1B), and pAG5018 (FIG. 1C).

FIG. 2 shows extracts from chymosin-expressing grain can coagulate milk. Tubes are numbered as described in examples herein.

FIG. 3 is photograph showing Western blot data for casein candidates.

FIGS. 4A-4C illustrate vectors containing expression cassettes for a mutated bovine myoglobin protein; the vectors are designated pAG5024 (FIG. 4A), pAG5025 (FIG. 4B), and pAG5026 (FIG. 4C).

FIGS. 5A-5C illustrate vectors containing expression cassettes for increasing heme biosynthesis in maize; the vectors are designated pAG5027 (FIG. 5A), pAG5028 (FIG. 5B) and pAG5029 (FIG. 5C).

FIGS. 6A-6H illustrate vectors containing the expression cassettes for myoglobin and EU591743 xylanase using aleurone specific promoters; the vectors are designated pAG5030 (FIG. 6A), pAG 5031 (FIG. 6B), pAG5032 (FIG. 6C), pAG5033 (FIG. 6D), pAG5034 (FIG. 6E), pAG5035 (FIG. 6F), pAG5036 (FIG. 6G), and pAG5037 (FIG. 6H).

FIG. 7 is a photograph of the Western blot that shows detection of myoglobin in extracts of seed derived from individual plants that had been transformed with T-DNAs carrying myoglobin expression constructs pAG5016, pAG5019 and pAG5020.

FIG. 8 is a photograph of the Western blot that shows detection of the mutant form of myoglobin in seed from plants that had been transformed with pAG5026 and pAG5019.

FIG. 9 is a photograph of the Western blot that shows the effect of pH on extraction of myoglobin from corn seed.

FIGS. 10A-10B are photographs of the Western blots that show the effect of detergent on myoglobin extraction efficiency.

FIG. 10A illustrates the effect the following extraction buffers: NaCarb, 30 mM sodium carbonate/bicarbonate, pH10.8; NaCarb Sarkosyl, 30 mM sodium carbonate/bicarbonate, pH10.8, 1% sarkosyl; TE Sarkosyl, 100 mM Tris 10 mM EDTA, pH8, 1% sarkosyl; TE Tween, 100 mM Tris 10 mM EDTA, pH8, 1% Tween-20; TE, 100 mM Tris 10 mM EDTA, pH8.

FIG. 10B illustrates the effect of the following extraction buffers: TE sarkosyl, 100 mM Tris 10 mM EDTA, pH8, 1% sarkosyl; Tris Sarkosyl, 100 mM Tris, pH8, 1% sarkosyl; TE Brij, 100 mM Tris 10 mM EDTA, pH8, 0.03% Brij-35; Tris Brij, 100 mM Tris, pH8, 0.03% Brij-35; Tris Triton, 100 mM Tris, pH8, 0.05% Triton X-100; Tris SDS, 100 mM Tris, pH8, 1% sodium dodecyl sulfate.

FIG. 11 are photographs that show quantitation of myoglobin in seed from pAG5026 candidates #31 and #36. The photograph to the left shows various amounts of purified myoglobin loaded as references; the photograph in the middle shows concentration of myoglobin in diluted samples of the candidate #31; and the photograph to the right shows concentration of myoglobin in diluted samples of the candidate #36.

FIG. 12 is a photograph that shows the effect of genes encoding heme biosynthetic enzymes on myoglobin accumulation.

### DETAILED DESCRIPTION OF THE PREFERRED EMBODIMENTS

Certain terminology is used in the following description for convenience only and is not limiting. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise.

The phrase “at least one” followed by a list of two or more items, such as “A, B, or C,” means any individual one of A, B or C as well as any combination thereof.

The words “right,” “left,” “top,” and “bottom” designate directions in the drawings to which reference is made.

Although methods and materials similar or equivalent to those described herein can be used in the practice or testing of this disclosure, suitable methods and materials are described below.

“Synthetic nucleic acid sequence,” “synthetic polynucleotide,” “synthetic oligonucleotide,” “synthetic DNA,” or “synthetic RNA” as used herein refers to a nucleic acid sequence, a polynucleotide, an oligonucleotide, DNA, or RNA that differs from one found in nature by having a different sequence that one found in nature or a chemical modification not found in nature. The definition of synthetic nucleic acid includes but is not limited to a DNA sequence created using biotechnology tools. Such tools include but are not limited to recombinant DNA technology, chemical synthesis, or directed use of nucleases (so called “genome editing” or “gene optimizing” technologies).

“Synthetic protein,” “synthetic polypeptide,” “synthetic oligopeptide,” “synthetic peptide”, “alternative protein”, “alternative polypeptide”, “alternative oligopeptide”, “alternative peptide”, “target protein”, “target polypeptide”, “target oligopeptide”, “target peptide”, “recombinant protein”, “recombinant polypeptide”, “recombinant oligopeptide”, “recombinant peptide”, as used herein refers to a protein, polypeptide, oligopeptide or peptide that was made through a synthetic process. The synthetic process includes but is not limited to chemical synthesis or recombinant technology.

As used herein, “variant” refers to a molecule that retains a biological activity that is the same or substantially similar to that of the original molecule. The variant may be from the same or different species or be a synthetic sequence based on a natural or prior molecule.

As used herein, the phrase “proteins of animal origin” refers to casein, chymosin, myoglobin, hemoglobin, and actin. This phrase also refers to variants, such as the mutant myoglobin protein.

“Casein” refers collectively to the commonly recognized family of casein molecules, including the alpha S1 (CasA1), alpha S2 (CasA2), beta (CasB), and kappa (CasK) proteins found in mammalian milk. “Casein” may refer to any one of these molecules, or all of them together, and may refer to variants that include signal peptides or tags for plant expression such as the gamma zein signal peptide and KDEL tag. Specific casein molecules, such as alpha S1, alpha S2, beta, and kappa, will be designated as such when discussing those specifically.

“Chymosin”, or renin, is a protease used in making cheese, where it cleaves casein. “Chymosin” refers collectively to both chymosin A and chymosin B, the latter of which is the more commonly used form of the enzyme for cheese making.

“Myoglobin” is heme-containing globular protein found in the cardiac and skeletal muscle tissue of vertebrates in general and in almost all mammals.

“Hemoglobin” is the iron-containing oxygen-transport protein present in red blood cells (erythrocytes) of almost all vertebrates as well as the tissues of some invertebrate animals. Myoglobin and hemoglobin are important nutritional sources of bioavailable iron, and the meat flavor in vegetarian foods. Myoglobin is associated with meat color, and for this reason is often added to plant-based meats.

“Actin” refers collectively to a family of globular multi-functional proteins that form the thin filaments in muscle fibrils. Actin is found in essentially all eukaryotic cells, where it may be present at a concentration of over 100 μM; its mass is roughly 42 kDa, with a diameter of 4 to 7 nm.

“Glutamyl-tRNA reductase” and “ferrochelatase” are enzymes participating in the biosynthesis of heme. “Glutamyl-tRNA reductase” is an enzyme that converts glutamyl-tRNA in an NADPH-dependent reaction into the labile intermediate glutamate-1-semialdehyde, which is a precursor to 5-aminolevulinate. “Ferrochelatase” catalyzes the terminal step in the biosynthesis of heme, converting protoporphyrin IX into heme B.

### Animal Proteins

In an embodiment, one or more myoglobin proteins are provided. The myoglobin protein may comprise an amino acid sequence with at least 70, 75, 80, 85, 90, 91, 92, 93, 94, 95, 96, 97, 98, 99, or 100% identity to a reference sequence selected from the group consisting of: SEQ ID NOS: 1-3, 20, 40 and 58. The myoglobin protein may comprise an amino acid sequence with at least 95% identity to a reference sequence selected from the group consisting of: SEQ ID NOS: 1-3, 20, 40 and 58. The myoglobin protein having less than 100% identity to its corresponding amino acid sequences of SEQ ID NO: 1-3, 20, 40 and 58 may be a variant of the referenced protein. In an embodiment, an isolated protein having a sequence with at least 70, 75, 80, 85, 90, 91, 92, 93, 94, 95, 96, 97, 98, 99, or 100% identity to a protein having the sequence of any one of SEQ ID NO: 1-3, 20, 40 and 58 along 7 to 10, 7 to 15, 7 to 30, 7 to 40, 7 to 50, 7 to 100, 7 to 150 or 7 to all amino acids of a protein having the sequence of any one of the SEQ ID NO: 1-3, 20, 40 and 58 are provided. The list of sequence lengths encompasses every full length protein in SEQ ID NO: 1-3, 20, 40 and 58 and every smaller length within the list, even for peptides that do not include over 50 amino acids. For example, the lengths of 7 to 10, 7 to 20, 7 to 30, 7 to 50, 7 to 100, 7 to 150, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NOS: 1-3, 20, 40 and 58 corresponding to the recited length of amino acids. A less than full length amino acid sequence may be selected from a portion of any one of SEQ ID NOS: 1-3, 20, 40 and 58.

In an embodiment, one or more hemoglobin proteins are provided. The hemoglobin protein comprising an amino acid sequence 70, 75, 80, 85, 90, 91, 92, 93, 94, 95, 96, 97, 98, 99, or 100% identity to a reference sequence selected from the group consisting of: SEQ ID NOS: 4-6, 21, and 41. The hemoglobin protein comprising an amino acid sequence 95% identity to a reference sequence selected from the group consisting of: SEQ ID NOS: 4-6, 21, and 41. The hemoglobin protein having less than 100% identity to its corresponding amino acid sequences of SEQ ID NO: 4-6, 21, and 41 may be a variant of the referenced protein. In an embodiment, an isolated protein having a sequence with at least 70, 75, 80, 85, 90, 91, 92, 93, 94, 95, 96, 97, 98, 99, or 100% identity to a protein having the sequence of any one of SEQ ID NO: 4-6, 21, and 41 along 7 to 10, 7 to 15, 7 to 30, 7 to 40, 7 to 50, 7 to 100, or 7 to all amino acids of a protein having the sequence of any one of the SEQ ID NO: 4-6, 21, and 41 are provided. The list of sequence lengths encompasses every full length protein in SEQ ID NO: 4-6, 21, and 41 and every smaller length within the list, even for peptides that do not include over 50 amino acids. For example, the lengths of 7 to 10, 7 to 20, 7 to 30, 7 to 50, 7 to 100, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NOS: 4-6, 21, and 41 corresponding to the recited length of amino acids. A less than full length amino acid sequence may be selected from a portion of any one of SEQ ID NOS: 4-6, 21, and 41.

In an embodiment, one or more actin proteins are provided. The actin protein may comprise an amino acid sequence with at least 70, 75, 80, 85, 90, 91, 92, 93, 94, 95, 96, 97, 98, 99, or 100% identity to a reference sequence selected from the group consisting of: SEQ ID NOS: 7-9, 22, and 42. The actin protein may comprise an amino acid sequence with at least 95% identity to a reference sequence selected from the group consisting of: SEQ ID NOS: 7-9, 22, and 42. The actin protein having less than 100% identity to its corresponding amino acid sequences of SEQ ID NO: 7-9, 22, and 42 may be a variant of the referenced protein. In an embodiment, an isolated protein having a sequence with at least 70, 75, 80, 85, 90, 91, 92, 93, 94, 95, 96, 97, 98, 99, or 100% identity to a protein having the sequence of any one of SEQ ID NO: 7-9, 22, and 42 along 7 to 10, 7 to 15, 7 to 30, 7 to 40, 7 to 50, 7 to 100, 7 to 150, 7 to 200, 7 to 250, 7 to 300, or 7 to all amino acids of a protein having the sequence of any one of the SEQ ID NO: 7-9, 22, and 42 are provided. The list of sequence lengths encompasses every full length protein in SEQ ID NO: 7-9, 22, and 42 and every smaller length within the list, even for peptides that do not include over 50 amino acids. For example, the lengths of 7 to 10, 7 to 20, 7 to 30, 7 to 50, 7 to 100, 7 to 150, 7 to 200, 7 to 250, 7 to 300, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NOS: 7-9, 22, and 42 corresponding to the recited length of amino acids. A less than full length amino acid sequence may be selected from a portion of any one of SEQ ID NOS: 7-9, 22, and 42.

In an embodiment, one or more casein proteins are provided. The casein protein may comprise an amino acid sequence with at least 70, 75, 80, 85, 90, 91, 92, 93, 94, 95, 96, 97, 98, 99, or 100% identity to a reference sequence selected from the group consisting of: SEQ ID NOS: 10-13, 15-18, and 35-38. The casein protein may comprise an amino acid sequence with at least 95% identity to a reference sequence selected from the group consisting of: SEQ ID NOS: 10-13, 15-18, and 35-38. The casein protein having less than 100% identity to its corresponding amino acid sequences of SEQ ID NO: 10-13, 15-18, and 35-38 may be a variant of the referenced protein. In an embodiment, an isolated protein having a sequence with at least 70, 75, 80, 85, 90, 91, 92, 93, 94, 95, 96, 97, 98, 99, or 100% identity to a protein having the sequence of any one of SEQ ID NO: 10-13, 15-18, and 35-38 along 7 to 10, 7 to 15, 7 to 30, 7 to 40, 7 to 50, 7 to 100, 7 to 150, or 7 to all amino acids of a protein having the sequence of any one of the SEQ ID NO: 10-13, 15-18, and 35-38 are provided. The list of sequence lengths encompasses every full length protein in SEQ ID NO: 10-13, 15-18, and 35-38 and every smaller length within the list, even for peptides that do not include over 50 amino acids. For example, the lengths of 7 to 10, 7 to 20, 7 to 30, 7 to 50, 7 to 100, 7 to 150, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NOS: 10-13, 15-18, and 35-38 corresponding to the recited length of amino acids. A less than full length amino acid sequence may be selected from a portion of any one of SEQ ID NOS: 10-13, 15-18, and 35-38.

In an embodiment, one or more chymosin proteins are provided. The chymosin protein may comprise an amino acid sequence with at least 70, 75, 80, 85, 90, 91, 92, 93, 94, 95, 96, 97, 98, 99, or 100% identity to a reference sequence selected from the group consisting of: SEQ ID NOS: 14, 19, and 39. The chymosin protein may comprise an amino acid sequence with at least 95% identity to a reference sequence selected from the group consisting of: SEQ ID NOS: 14, 19, and 39. The chymosin protein having less than 100% identity to its corresponding amino acid sequences of SEQ ID NO: 14, 19, and 39 may be a variant of the referenced protein. In an embodiment, an isolated protein having a sequence with at least 70, 75, 80, 85, 90, 91, 92, 93, 94, 95, 96, 97, 98, 99, or 100% identity to a protein having the sequence of any one of SEQ ID NO: 14, 19, and 39 along 7 to 10, 7 to 15, 7 to 30, 7 to 40, 7 to 50, 7 to 100, 7 to 150, 7 to 200, 7 to 250, 7 to 300, or 7 to all amino acids of a protein having the sequence of any one of the SEQ ID NO: 14, 19, and 39 are provided. The list of sequence lengths encompasses every full length protein in SEQ ID NO: 14, 19, and 39 and every smaller length within the list, even for peptides that do not include over 50 amino acids. For example, the lengths of 7 to 10, 7 to 20, 7 to 30, 7 to 50, 7 to 100, 7 to 150, 7 to 200, 7 to 250, 7 to 300, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NOS: 14, 19, and 39 corresponding to the recited length of amino acids. A less than full length amino acid sequence may be selected from a portion of any one of SEQ ID NOS: 14, 19, and 39.

### Proteins Increasing Heme Biosynthesis

In an embodiment, one or more glutamyl-tRNA reductases are provided. The glutamyl-tRNA reductase may comprise an amino acid sequence with at least 70, 75, 80, 85, 90, 91, 92, 93, 94, 95, 96, 97, 98, 99, or 100% identity to a reference sequence of SEQ ID NO: 59. The glutamyl-tRNA reductase may comprise an amino acid sequence with at least 95% identity to a reference sequence of SEQ ID NO: 59. The glutamyl-tRNA reductase having less than 100% identity to its corresponding amino acid sequences of SEQ ID NO: 59 may be a variant of the referenced protein. In an embodiment, an isolated protein having a sequence with at least 70, 75, 80, 85, 90, 91, 92, 93, 94, 95, 96, 97, 98, 99, or 100% identity to a protein having the sequence of any one of SEQ ID NO: 59 along 7 to 10, 7 to 15, 7 to 30, 7 to 40, 7 to 50, 7 to 100, 7 to 150, 7 to 200, 7 to 250, 7 to 300, 7 to 350 or 7 to all amino acids of a protein having the sequence of any one of the SEQ ID NO: 59 are provided. The list of sequence lengths encompasses every full length protein in SEQ ID NO: 59 and every smaller length within the list, even for peptides that do not include over 50 amino acids. For example, the lengths of 7 to 10, 7 to 20, 7 to 30, 7 to 50, 7 to 100, 7 to 150, 7 to 200, 7 to 250, 7 to 300, 7 to 350, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NO: 59 corresponding to the recited length of amino acids. A less than full length amino acid sequence may be selected from a portion of any one of SEQ ID NO: 59.

In an embodiment, one or more ferrochelatases are provided. The ferrochelatase may comprise an amino acid sequence with at least 70, 75, 80, 85, 90, 91, 92, 93, 94, 95, 96, 97, 98, 99, or 100% identity to a reference sequence of SEQ ID NO: 61. The ferrochelatase may comprise an amino acid sequence with at least 95% identity to a reference sequence of SEQ ID NO: 61. The ferrochelatase having less than 100% identity to its corresponding amino acid sequences of SEQ ID NO: 61 may be a variant of the referenced protein. In an embodiment, an isolated protein having a sequence with at least 70, 75, 80, 85, 90, 91, 92, 93, 94, 95, 96, 97, 98, 99, or 100% identity to a protein having the sequence of any one of SEQ ID NO: 61 along 7 to 10, 7 to 15, 7 to 30, 7 to 40, 7 to 50, 7 to 100, 7 to 150, 7 to 200, 7 to 250, 7 to 300, 7 to 350 7 to 400 or 7 to all amino acids of a protein having the sequence of any one of the SEQ ID NO: 61 are provided. The list of sequence lengths encompasses every full length protein in SEQ ID NO: 61 and every smaller length within the list, even for peptides that do not include over 50 amino acids. For example, the lengths of 7 to 10, 7 to 20, 7 to 30, 7 to 50, 7 to 100, 7 to 150, 7 to 200, 7 to 250, 7 to 300, 7 to 350, 7 to 400, and 7 to all amino acids would apply to a sequence with 50 amino acids. A range of amino acid sequence lengths recited herein includes every length of amino sequence within the range, endpoints inclusive. The recited length of amino acids may start at any single position within a reference sequence where enough amino acids follow the single position to accommodate the recited length. The fragment may have 5, 7, 8, 9, 10, 15, 20, 25, 30, 35, 40, 45 or 50 amino acids. The fragments may include 5, 7, 8, 9, 10, 15, 20, 25, 30, 35, 40, 45 or 50 contiguous amino acids. Embodiments also include nucleic acids or polynucleotides, encoding said amino acid sequences. A less than full length amino acid sequence may be selected from any portion of one of the sequences of SEQ ID NO: 61 corresponding to the recited length of amino acids. A less than full length amino acid sequence may be selected from a portion of any one of SEQ ID NO: 61.

### Nucleic Acids Encoding Animal Proteins

An embodiment provides one or more nucleic acids encoding the myoglobin protein or its variants described herein. The one or more nucleic acids may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 32, or 78. The one or more nucleic acids may be included in the expression cassette to be expressed in a host. The host may be but is not limited to a microorganism, a plant cell, a phage, a virus, a mammalian cell, or an insect cell. The one or more nucleic acids may be codon optimized for expression in the host. The one or more nucleic acids may be codon optimized for plant expression.

An embodiment provides one or more nucleic acids encoding the actin protein or its variants described herein. The one or more nucleic acids may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 34.

An embodiment provides one or more nucleic acids encoding the casein proteins or their variants described herein. The one or more nucleic acids may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 27-30.

An embodiment provides one or more nucleic acids encoding the chymosin proteins or its variants described herein. The one or more nucleic acids may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 31.

An embodiment provides one or more nucleic acids the hemoglobin proteins or its variants described herein. The one or more nucleic acids may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 33.

### Nucleic Acids Encoding Proteins Increasing Heme Biosynthesis

An embodiment provides one or more nucleic acids encoding the glutamyl-tRNA reductase or its variants described herein. The one or more nucleic acids may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 79.

An embodiment provides one or more nucleic acids encoding the ferrochelatase or its variants described herein. The one or more nucleic acids may comprise, consist essentially of, or consist of a sequence with at least 70, 72, 75, 80, 85, 90, 91, 92, 93, 94, 95, 96, 97, 98, 99, or 100% identity to a reference sequence of SEQ ID NO: 81.

The one or more nucleic acids may be included in the expression cassette to be expressed in a host. The host may be but is not limited to a microorganism, a plant cell, a phage, a virus, a mammalian cell, or an insect cell. The one or more nucleic acids may be codon optimized for expression in the host. The one or more nucleic acids may be codon optimized for plant expression.

### Expression Cassettes

An embodiment provides an expression cassette comprising a synthetic polynucleotide that comprises one or more nucleic acids encoding the animal-derived proteins described herein.

A polynucleotide sequence in an expression cassette, isolated nucleic acid, vector, or any other DNA construct herein, or utilized in a method herein may be operably connected to one or more regulatory elements. A regulatory element included may be a promoter. The promoter may be a constitutive promoter that provides transcription of the polynucleotide sequences throughout the plant in most cells, tissues and organs and during many but not necessarily all stages of development. The promoter may be an inducible promoter, which initiates transcription of the polynucleotide sequences only when exposed to a particular chemical or environmental stimulus. The promoter may be specific to a host. The promoter may be suitable for expression of the polynucleotide in a plant, a bacterium, yeast, a mammalian cell, or an insect cell. The promoter may be a plant specific promoter. The promoter may be specific to a particular developmental stage, organ, tissue, or derived from a specific plant species. A tissue specific promoter may be capable of initiating transcription in a particular plant tissue. Plant tissue that may be targeted by a tissue specific promoter may be but is not limited to a stem, leaves, trichomes, anthers, pollen, seed, embryo, or endosperm. A constitutive promoter herein may be the maize Ubiquitin promoter, the rice Ubiquitin 3 promoter (OsUbi3P), the switchgrass ubiquitin promoter, the PEPC promoter, the maize Actin promoter, or the rice Actin 1 promoter. The constitutive promoter may be an aleurone specific promoter. The aleurone specific promoter may be a maize A19 prZmAI9 promoter, or barley Ltp2 gene HvLtp2 promoter. The promoter may comprise a nucleic acid sequence with at least 70, 72, 75, 80, 85, 90, 91, 92, 93, 94, 95, 96, 97, 98, 99, or 100% identity to a reference of SEQ ID NO: 83 or 84.

Other known constitutive promoters may be used, and include but are not limited to Cauliflower Mosaic Virus (CAMV) 35S promoter, the Cestrum Yellow Leaf Curling Virus promoter (CMP) or the CMP short version (CMPS), and the Rubisco small subunit promoter.

The tissue specific promoter may include the seed-specific promoter. The seed-specific promoter may be an embryo-specific promoter or an endosperm-specific promoter. The seed specific promoter may be but is not limited to the maize zein promoter, the rice glutelin (GluB4) promoter, the maize oleosin promoter, or the maize globulin promoter or soybean alpha′ subunit beta-conglycinin promoter. The promoter may be a soybean prGmCG1 promoter. The promoter may comprise a nucleic acid sequence with at least 70, 72, 75, 80, 85, 90, 91, 92, 93, 94, 95, 96, 97, 98, 99, or 100% identity to a reference of SEQ ID NO: 85, 87 or 88.

The promoter may be a promoter homolog to any one of the previously listed promoters derived from other species, or promoter variants to the previously listed promoters with greater than 80% identity.

The promoter may be suitable for expressing the one or more polynucleotides in a bacterium. The promoter may be the T7 RNA polymerase promoter, the LAC promoter or the arabinose promoter. The promoter may be suitable for expressing the polynucleotide in a yeast. The promoter may be the GAL promoter or the glucose promoter. The promoter may be any prokaryotic promoter. The prokaryotic promoter may be a bacterial promoter, or phage promoter that is active in bacteria. The prokaryotic promoter may be any inducible promoter that is active in bacteria, or any other promoter that is active in bacteria.

Another regulatory element that may be provided is a terminator sequence, which terminates transcription. A terminator sequence may be included at the 3′ end of a transcriptional unit of the expression cassette. The terminator may be derived from a variety of genes. The terminator may be from a eukaryote, such as a plant or mammalian cell, or a prokaryote. The terminator may be a terminator sequence from the nopaline synthase oroctopine synthase genes of Agrobacterium tumefaciens. The terminator may be a terminator from Cauliflower Mosaic Virus (CaMV) 35S. The terminator may be a maize gamma zein 27 terminator. The terminator may be a maize A19 terminator. The terminator may comprise a nucleic acid sequence with at least 70, 72, 75, 80, 85, 90, 91, 92, 93, 94, 95, 96, 97, 98, 99, or 100% identity to a reference sequence of SEQ ID NO: 86. The terminator may be any other terminator sequence.

The one or more synthetic polynucleotide may further include one or more signal polynucleotide sequence encoding any one of the signal peptides described herein. The signal polynucleotide may comprise a nucleic acid sequence with at least 70, 72, 75, 80, 85, 90, 91, 92, 93, 94, 95, 96, 97, 98, 99, or 100% identity to a reference sequence selected from the group consisting of SEQ ID NOS: 24, 26, 80, and 82.

The expression cassette may comprise, consist essentially of, or consist of a synthetic polynucleotide sequence with at least 70, 72, 75, 80, 85, 90, 91, 92, 93, 94, 95, 96, 97, 98, 99, or 100% identity to a reference sequence of SEQ ID NO: 43-52, 56 and 64-77.

In an embodiment, the synthetic polynucleotide may comprise one or more nucleic acids encoding a myoglobin protein or its variants described herein. The synthetic polynucleotide may comprise a sequence with at least 70, 75, 80, 85, 90, 91, 92, 93, 94, 95, 96, 97, 98, 99, or 100% identity to a reference sequence selected from the group consisting of: SEQ ID NO: 48, 51-52, 64-66, 70, 72, and 74-77.

In an embodiment, the synthetic polynucleotide may comprise one or more nucleic acids encoding the hemoglobin proteins or their variants described herein. The synthetic polynucleotide may comprise a sequence with at least 70, 75, 80, 85, 90, 91, 92, 93, 94, 95, 96, 97, 98, 99, or 100% identity to the sequence of SEQ ID NO: 49.

In an embodiment, the synthetic polynucleotide may comprise one or more nucleic acids encoding proteins that increase availability of heme in seed endosperm. The synthetic polynucleotide may comprise one or more nucleic acids encoding the glutamyl-tRNA reductase or its variants described herein. The synthetic polynucleotide may comprise a sequence with at least 70, 75, 80, 85, 90, 91, 92, 93, 94, 95, 96, 97, 98, 99, or 100% identity to a reference sequence selected from the group consisting of: SEQ ID NO: 67.

The synthetic polynucleotide may comprise one or more nucleic acids encoding the ferrochelatase. The synthetic polynucleotide may comprise a sequence with at least 70, 75, 80, 85, 90, 91, 92, 93, 94, 95, 96, 97, 98, 99, or 100% identity to a reference sequence selected from the group consisting of: SEQ ID NO: 68 or 69.

In an embodiment, the synthetic polynucleotide may comprise one or more nucleic acids encoding an actin protein or its variants described herein. The synthetic polynucleotide may comprise a sequence with at least 70, 75, 80, 85, 90, 91, 92, 93, 94, 95, 96, 97, 98, 99, or 100% identity to the sequence of SEQ ID NO: 50.

In an embodiment, the synthetic polynucleotide may comprise one or more nucleic acids encoding the casein proteins or their variants described herein. The synthetic polynucleotide may comprise a sequence with at least 70, 75, 80, 85, 90, 91, 92, 93, 94, 95, 96, 97, 98, 99, or 100% identity to the sequence selected from the group consisting of SEQ ID NO: 44-47.

In an embodiment, the synthetic polynucleotide may comprise one or more nucleic acids encoding the chymosin proteins or their variants described herein. The synthetic polynucleotide may comprise a sequence with at least 70, 75, 80, 85, 90, 91, 92, 93, 94, 95, 96, 97, 98, 99, or 100% identity to the sequence of SEQ ID NO: 43.

An embodiment provides a synthetic polynucleotide encoding at least one expression cassette for producing at least one animal-derived protein to be expressed in a transgenic plant.

The expression cassette may be a cassette for producing at least one myoglobin protein. The expression cassette may be a cassette for producing at least one hemoglobin protein. The expression cassette may be a cassette for producing at least one chymosin protein. The expression cassette may be a cassette for producing at least one casein protein. The expression cassette may be a cassette for producing at least one actin protein.

The expression cassette may be a cassette for expression two or more animal proteins described herein. The expression cassette may be a cassette for producing at least ono hemoglobin and at least one myoglobin proteins. The expression cassette may be a cassette for producing at least ono casein and at least one chymosin proteins. The expression cassette may be a cassette for producing any one of the animal-derived proteins in any combination.

The expression cassette including the one or more synthetic polynucleotides may be included in a vector.

### Vectors

An embodiment comprises a vector containing the expression cassette including one or more synthetic polynucleotides encoding a myoglobin, actin, casein, or chymosin protein of any of the above embodiments. The vector may contain any one of the expression cassettes described in any of the embodiments herein. The vector may be a vector used in plant transformation and that can deliver its DNA into the genome of plant cells. The vector may be a vector used for yeast and fungal expression. The vector may be a vector for expression of the peptides, concatenated peptides or antibodies described herein used in bacterial expression. The vector may be a vector used for mammalian or insect cell expression.

An embodiment comprises a polynucleotide comprising, consisting essentially of, or consisting of a sequence that has at least 70, 72, 75, 80, 85, 90, 91, 92, 93, 94, 95, 96, 97, 98, 99, or 100% identity along its length to a contiguous portion of a polynucleotide having any one of the sequences set forth herein or the complements thereof. The contiguous portion may be any length up to the entire length of a sequence set forth herein or the complement thereof.

Determining percent identity of two amino acid sequences or two nucleic acid sequences may include aligning and comparing the amino acid residues or nucleotides at corresponding positions in the two sequences. If all positions in two sequences are occupied by identical amino acid residues or nucleotides then the sequences are said to be 100% identical. Percent identity is measured by the Smith Waterman algorithm (Smith T F, Waterman M S 1981 “Identification of Common Molecular Subsequences,” J Mol Biol 147: 195-197, which is incorporated herein by reference as if fully set forth).

### Transgenic Plants

In an embodiment, a transgenic plant comprising any one of the nucleic acids and synthetic polynucleotides described herein and expressing any one of the myoglobin, hemoglobin, actin, chymosin, casein, glutamyl-tRNA reductase, ferrochelatase or proteins described herein is provided. As used herein, the term “transgenic plants” describes plants transformed with DNA that enables the plant containing the transformed DNA to perform a novel function; usually the transcription of the DNA, potentially at a level different from the level in wild-type plants, and potentially the translation of the transcript into a protein, which may be a novel protein to the plant. The transgenic plant may refer to a whole transgenic plant or tissues thereof. The tissues of transgenic plants may be any portion of a transgenic plant, including but not limited to leaves, stems, flowers, buds, petals, grain, seed, embryo, endosperm, leaves, stalks, roots, pollen, or anthers. The tissues may also refer to liquid extracts made by fractionating any portion of a transgenic plant in an organic or aqueous liquid (for example, extracting protein from transgenic seeds or grain and using the extract as a source of the transgenic protein). The tissue may be callus from a transgenic plant. The tissue may be seeds from a transgenic plant that accumulate peptides, myoglobin, actin, chymosin, or casein proteins described herein. A transgenic plant may be regenerated from tissues of a transgenic plant. A transgenic plant may be a product of sexual crossing of a first transgenic plant and a second transgenic plant, or a non-transgenic plant, where the product plant retains an engineered nucleic acid introduced to the first transgenic plant. A transgenic plant may be a product of self-pollination of a first transgenic plant with itself.

A transgenic plants may be a monocotyledonous plant. As used herein, the term “monocotyledonous plant,” or “monocot,” refers to grass and grass-like flowering plants (angiosperms), the seeds of which typically contain only one embryonic leaf, or cotyledon. The monocotyledonous plants may include, but not be limited to, maize, rice, wheat, sorghum, switchgrass, millet, sugarcane, or other plants.

In an embodiment, the transgenic plant may be a corn plant. The tissue of the corn plant may be corn grain. In an embodiment, the at least one animal-derived protein may be expressed in corn grain. The at least one animal-derived protein may be expressed in any portion of the corn grain. For example, the at least one animal-derived protein may be expressed in an endosperm of the corn grain. Alternatively, the at least one animal-derived protein may be expressed in an embryo of the corn grain.

In an embodiment, the at least one animal-derived protein may be expressed in the corn grain at a level in the range from 0.01 mg to 24.0 mg of a recombinant protein per gram of grain. The at least one animal-derived protein may be expressed at a level of at least 0.01 mg, 0.10 mg, 1.0 mg, 2.0 mg, 3.0 mg, 4.0 mg, 5.0 mg, 6.0 mg, 7.0 mg, 8.0 mg. 9.0 mg, 10.0 mg, 11.0 mg, 12.0 mg, 13.0 mg, 14.0 mg, 15.0 mg, 16.0 mg, 17.0 mg, 18.0 mg, 19.0 mg, 20.0 mg, 21.0 mg, 22.0 mg, 23.0 mg, 24.0 mg, or any value between any two of the foregoing expression level points of a recombinant protein per gram of grain.

In an embodiment, the at least one animal-derived protein may be expressed in the endosperm of the corn grain at a level in the range from 0.01 mg to 24.0 mg of a recombinant protein per gram of grain. The at least one animal-derived protein may be expressed at a level of at least 0.01 mg, 0.10 mg, 1.0 mg, 2.0 mg, 3.0 mg, 4.0 mg, 5.0 mg, 6.0 mg, 7.0 mg, 8.0 mg. 9.0 mg, 10.0 mg, 11.0 mg, 12.0 mg, 13.0 mg, 14.0 mg, 15.0 mg, 16.0 mg, 17.0 mg, 18.0 mg, 19.0 mg, 20.0 mg, 21.0 mg, 22.0 mg, 23.0 mg, 24.0 mg, or any value between any two of the foregoing expression level points of a recombinant protein per gram of grain.

In an embodiment, the at least one animal-derived protein may be expressed in the embryo of the corn grain at a level in the range from 0.01 mg to 24.0 mg of a recombinant protein per gram of grain. The at least one animal-derived protein may be expressed at a level of at least 0.01 mg, 0.10 mg, 1.0 mg, 2.0 mg, 3.0 mg, 4.0 mg, 5.0 mg, 6.0 mg, 7.0 mg, 8.0 mg. 9.0 mg, 10.0 mg, 11.0 mg, 12.0 mg, 13.0 mg, 14.0 mg, 15.0 mg, 16.0 mg, 17.0 mg, 18.0 mg, 19.0 mg, 20.0 mg, 21.0 mg, 22.0 mg, 23.0 mg, 24.0 mg, or any value between any two of the foregoing expression level points of a recombinant protein per gram of grain.

In an embodiment, the at least one animal-derived protein may be expressed in the aleurone cells of the corn grain at a level in the range from 0.01 mg to 24.0 mg of a recombinant protein per gram of grain. The at least one animal-derived protein may be expressed at a level of at least 0.01 mg, 0.10 mg, 1.0 mg, 2.0 mg, 3.0 mg, 4.0 mg, 5.0 mg, 6.0 mg, 7.0 mg, 8.0 mg. 9.0 mg, 10.0 mg, 11.0 mg, 12.0 mg, 13.0 mg, 14.0 mg, 15.0 mg, 16.0 mg, 17.0 mg, 18.0 mg, 19.0 mg, 20.0 mg, 21.0 mg, 22.0 mg, 23.0 mg, 24.0 mg, or any value between any two of the foregoing expression level points of a recombinant protein per gram of grain.

A transgenic plants may be a dicotyledonous plant. As used herein, the term “dicotyledonous plant,” or “dicot,” refers to the group of flowering plants, the seed of which has two embryonic leaves or cotyledons. The dicotyledonous plants may include, but not be limited to, soybeans, legumes (e.g., pea, beans, lentils, peanuts), sugar beet, cabbage, potato, tomato or other plants.

An embodiment provides a progeny of any one of the transgenic plants described herein. The transgenic plant may express any one of the myoglobin, actin, chymosin, or casein proteins described herein. The targeted endogenous molecules may be peptides or related molecules. The transgenic plant may contain at least one of the expression cassettes that are described herein. The transgenic plant may be produced using the vectors described herein. The transgenic plant may be capable of producing any one of the peptides, myoglobin, actin, chymosin, or casein proteins described herein. The transgenic plant expressing peptides, myoglobin, actin, chymosin, or casein proteins described herein, may be but is not limited to tobacco plant, corn plants, soy bean plants, or any other plant commonly eaten by animals.

### Methods of Making Transgenic Plants

In an embodiment, a method of making any one of the transgenic plants described herein is provided. The method may comprise culturing explants from a target plant and contacting them with a vector that contains at least one expression cassette described herein. The target plant may be a corn or soy bean plant, or it may be pea, wheat, rice, sorghum, tobacco, canola, cotton, switchgrass, or another plant. The method may include contacting the vector with the plant explant, for example, by using biolistic transformation or by using Agrobacterium transformation. Once the explant has been contacted by the vector, methods of selecting and regenerating whole plants may be used that are known in the art.

### Processes for Isolating Animal Proteins

In an embodiment, any one of the myoglobin, hemoglobin, actin, chymosin, or casein proteins may be isolated from the transgenic plant or plant tissue described herein.

In an embodiment, the specific recombinant or engineered molecules described herein may be isolated from other expression hosts.

In an embodiment, a process for isolating any one of the animal-derived proteins from a transgenic plant or tissues thereof described herein is provided.

The process may be a modified grain process that uses grain as a raw material to make other products. The modified grain process may be a modified wet milling, dry milling, or dry-grind process. Dry milling typically uses a mill to reduce the size of grain or other plant material. For example, grains expressing animal-derived proteins may be milled to produce flour prior to further use of buffer extraction of the animal-derived proteins. Wet milling, also known as wet grinding, is a process through which particles that are suspended in a liquid slurry are subsequently reduced in size by shearing or crushing. This process may include steeping of the plant material prior to protein isolation.

Once the milling process is complete, these particles are ready for use or can be dried and separated for incorporation into additional products.

In an embodiment, the isolation may be performed by using an organic solvent extraction. Alternatively, the isolation may be performed by using an aqueous extraction. The aqueous extraction may be performed by using an aqueous buffer. The aqueous buffer may comprise a detergent. The aqueous buffer may be selected from the group consisting of: Britton-Robinson (BR) polybuffer (Britton & Robinson, 1931), pH4 to pH9; deionized water; 100 mM Tris, 10 mM EDTA; 100 mM sodium phosphate, pH6.5; 30 mM sodium carbonate/bicarbonate, pH10.8. The aqueous buffer may comprise a detergent. The detergent may be selected from the group consisting of: Sarkosyl, Tween, or Sodium Dodecyl Sulfate. The concentration of the detergent may be 3% (v/v), 2.5% (v/v), 2.0 (v/v), 1.5 (v/v), 1.0 (v/v), 0.5 (v/v) or 0.01% (v/v), or any value between any two of the foregoing concentration points of the detergent per buffer. The percent detergent listed here refers to the percent of stock detergent in comparison to the final volume of the aqueous buffer.

In an embodiment, the isolation may be performed from the transgenic monocotyledonous plant. For example, the isolation may be performed from a corn plant following milling of the corn grain. Depending on the promoter used for production of the transgenic corn plant, the one or more animal-derived proteins may be expressed in an endosperm or embryo of the corn grain. If the animal-derived proteins are expressed in the endosperm, the process may comprise the step of deembryonization or degermination of the corn grain. The subsequent isolation may be performed from the remaining fiber and endosperm. The isolation of the protein may also occur by extraction from the corn gluten feed, or from the corn gluten meal.

Following the extraction, the animal-derived protein may be further purified using heating of the extract, centrifugation, evaporation, filtration, pressed to remove liquid, spray drying, or drying.

Thus isolated animal-derived proteins, i.e., myoglobin, actin, chymosin, casein and/or hemoglobin proteins, may have improved thermal stability and may remain soluble after being exposed to elevated temperatures above room temperature up to 40° C.

### Plant-Based Meat Compositions

An embodiment provides a plant-based meat composition comprising any one of animal-derived proteins described herein, or any combination thereof. The plant-based meat composition may comprise any of the transgenic plants or tissues thereof comprising animal derived proteins described herein or any combination thereof. The plant-based meat composition may comprise any one of the animal-derived proteins isolated from the transgenic plants or tissues thereof described herein. The plant-based meat composition may comprise any one of the myoglobin proteins isolated from the transgenic plants or tissues thereof described herein. The plant-based meat composition may comprise any one of the hemoglobin proteins isolated from the transgenic plants or tissues thereof described herein. The plant-based meat composition may comprise any one of the actin proteins isolated from the transgenic plants or tissues thereof described herein. The plant-based meat composition may comprise a combination of any one of the hemoglobin and myoglobin proteins isolated from the transgenic plants or tissues thereof described herein. The plant-based meat composition may comprise a combination of any one of the hemoglobin, myoglobin and actin proteins isolated from the transgenic plants or tissues thereof described herein. The plant-based meat composition may comprise a casein protein isolated from the transgenic plants or tissues described herein. The plant-based meat composition may comprise a chymosin protein isolated from the transgenic plants or tissues described herein.

The plant-based meat composition may comprise a combination of any one of the myoglobin and animal-derived proteins isolated from the transgenic plants or tissues thereof described herein.

The term “plant-based meat” refers to a composition comprising plant tissue prepared to resemble animal tissue when used in food. The plant-based meat compositions may comprise additives, e.g., thickening stabilizers, starches, water-soluble soybean polysaccharides, and/or preservatives. The plant-based meat composition may comprise one or more gelling agents, such as guar gum, xanthan gum, locust bean gum, or agar. The plant-based meat composition may comprise emulsifiers such as lecithin, fatty acid esters and organic monoglycerides. The plant-based meat composition may comprise plant or vegetable oils that include, but are not limited to, olive oil, canola oil, sunflower oil, flax seed oil, grape seed oil, peanut oil, or any other oil derived from plant sources. The plant-based meat compositions may further contain seasonings, sodium glutamate, spices, plant derived purees and powders. The plant-based meat composition may comprise coloring agents. The plant-based meat composition may have an appearance and flavor of a meat cut, meat patty or ground meat.

### Plant-Based Cheese Compositions

An embodiment provides a plant-based cheese composition comprising at least one of any of the transgenic plants or tissues thereof described herein.

In an embodiment, the plant-based cheese composition may comprise any of the casein proteins isolated from the transgenic plants or tissues thereof described herein. In an embodiment, the plant-based cheese composition may comprise any of the chymosin proteins isolated from the transgenic plants or tissues thereof described herein. In an embodiment, the plant-based cheese composition may comprise a combination of the chymosin or casein proteins isolated from the transgenic plants or tissues thereof described herein. The plant-based cheese composition may also comprise a myoglobin protein isolated from the transgenic plants or tissues thereof described herein. The plant-based cheese composition may comprise an actin protein isolated from the transgenic plants or tissues thereof described herein. The plant-based cheese composition may comprise a hemoglobin protein isolated from the transgenic plants or tissues thereof described herein.

In an embodiment, the plant-based cheese composition may contain additives, e.g., thickening stabilizers, starches, water-soluble soybean polysaccharides, and/or preservatives. The composition may comprise one or more gelling agents, such as guar gum, xanthan gum, locust bean gum, or agar. The composition may comprise emulsifiers such as lecithin, fatty acid esters ad organic monoglycerides. The compositions may further contain seasoning sodium glutamate, spices, milk flavor agents, plant derived purees and powders, sweeteners such as glucose, sucrose, aspartame, stevia. The plant-based cheese compositions may be hard cheeses, spreads, fillings, fresh creams, cheese cakes or puddings.

### Other Food or Feed Compositions

An embodiment provides a food or feed composition comprising any one of animal-derived proteins described herein, or any combination thereof. The food or feed composition may comprise any of the transgenic plants or tissues thereof comprising animal derived proteins described herein or any combination thereof. The food or feed composition may comprise any one of the animal-derived proteins isolated from the transgenic plants or tissues thereof described herein. The food or feed composition may comprise any one of the myoglobin proteins isolated from the transgenic plants or tissues thereof described herein. The food or feed composition may comprise any one of the hemoglobin proteins isolated from the transgenic plants or tissues thereof described herein. The food or feed composition may comprise any one of the actin proteins isolated from the transgenic plants or tissues thereof described herein. The food or feed composition may comprise a combination of any one of the hemoglobin and myoglobin proteins isolated from the transgenic plants or tissues thereof described herein. The food or feed composition may comprise a combination of any one of the hemoglobin, myoglobin and actin proteins isolated from the transgenic plants or tissues thereof described herein. The food or feed composition may comprise a casein protein isolated from the transgenic plants or tissues described herein. The food or feed composition may comprise a chymosin protein isolated from the transgenic plants or tissues described herein.

The food or feed composition may comprise a combination of any one of the myoglobin and animal-derived proteins isolated from the transgenic plants or tissues thereof described herein.

The term “food or feed” refers to a composition comprising plant tissue prepared to be eaten by humans or animals. The food or feed compositions may comprise additives, e.g., thickening stabilizers, starches, water-soluble soybean polysaccharides, and/or preservatives. The food or feed composition may comprise one or more gelling agents, such as guar gum, xanthan gum, locust bean gum, collagen, collagen peptides, gelatin, or agar. The food or feed composition may comprise emulsifiers such as lecithin, fatty acid esters and organic monoglycerides. The food or feed composition may comprise plant or vegetable oils that include, but are not limited to, olive oil, canola oil, sunflower oil, flax seed oil, grape seed oil, peanut oil, or any other oil derived from plant sources. The food or feed compositions may further contain seasonings, sodium glutamate, spices, plant derived purees and powders. The food or feed composition may comprise coloring agents. The food or feed composition may have an appearance and flavor of a meat cut, meat patty or ground meat.

The following list includes particular embodiments of the present invention. But the list is not limiting and does not exclude alternate embodiments, or embodiments otherwise described herein. Percent identity described in the following embodiments list refers to the identity of the recited sequence along the entire length of the reference sequence.

### EMBODIMENTS

1. A transgenic plant or tissue thereof comprising a synthetic polynucleotide encoding a myoglobin protein.

2. The transgenic plant or tissues thereof of embodiment 1, wherein the transgenic plant is a dicotyledonous plant.

3. The transgenic plant or tissues thereof of one or both embodiments 1 and 2, wherein the dicotyledonous plant is selected from the group consisting of soybeans, legumes, such as pea, beans, lentils, and peanuts, sugar beet, cabbage, potato, and tomato.

4. The transgenic plant or tissues thereof of embodiment 1, wherein the transgenic plant is a monocotyledonous plant.

5. The transgenic plant or tissues thereof of any one or both embodiments 1 and 4, wherein the monocotyledonous plant is selected from the group consisting of: corn, rice, wheat, oat, barley and millet.

6. The transgenic plant or tissues thereof any one or more of embodiments 1 and 4-5, wherein the tissue of the monocotyledonous is grain.

7. The transgenic plant or tissues thereof any one or more of embodiments 1 and 4-6, wherein the myoglobin protein is expressed at a level in a range from 0.01 mg to 24.0 mg of the myoglobin protein per gram of grain.

8. The transgenic plant or tissues thereof any one or more of embodiments 1 and 4-7, wherein the monocotyledonous plant is corn, and the tissue thereof is a corn grain.

9. The transgenic plant or tissues thereof of any one or more of embodiments 1 and 4-8, wherein myoglobin protein is expressed in the corn grain at a level in the range from 0.01 mg to 24.0 mg of the myoglobin protein per gram of corn grain.

10. The transgenic plant or tissues thereof of any one or more of embodiments 1 and 4-9, wherein the myoglobin protein is expressed in an endosperm or aleurone cells of the corn grain.

11. The transgenic plant or tissues thereof any one or more of embodiments 1 and 4-10, wherein the myoglobin protein is expressed in the endosperm or aleurone cells of the corn grain at a level in the range from 0.01 mg to 24.0 mg of the animal-derived protein per gram of corn grain.

12. The transgenic plant or tissues thereof of any one or more of embodiments 1 and 4-9, wherein myoglobin protein is expressed in an embryo of the corn grain.

13. The transgenic plant or tissues thereof of any one or more of embodiments 1, 4-9 and 12, wherein the myoglobin protein is expressed in the embryo of the corn grain at a level in the range from 0.01 mg to 24.0 mg of the myoglobin protein per gram of grain.

14. The transgenic plant or tissues thereof of any one or more of embodiments 1-13, wherein the nucleic acid encoding the myoglobin protein comprises a sequence with at least 90% identity to a reference sequence of SEQ ID NO: 32 or 78.

15. The transgenic plant or tissues thereof of any one or more of embodiments 1-14, wherein the myoglobin protein comprises an amino acid sequence with at least 95% identity to a reference sequence selected from the group consisting of: SEQ ID NOS: 1-3, 20, 40 and 58.

16. A transgenic plant or tissue thereof comprising a synthetic polynucleotide encoding at least one animal-derived protein, wherein the animal-derived protein is selected from the group consisting of a myoglobin, hemoglobin, actin, chymosin, and casein proteins.

17. The transgenic plant or tissues thereof of embodiment 16, wherein the transgenic plant is a dicotyledonous plant.

18. The transgenic plant or tissues thereof of any one or both embodiments 16 and 17, wherein the dicotyledonous plant is selected from the group consisting of soybeans, legumes, such as pea, beans, lentils, and peanut, sugar beet, cabbage, potato, and tomato.

19. The transgenic plant or tissues thereof of embodiment 16, wherein the transgenic plant is a monocotyledonous plant.

20. The transgenic plant or tissues thereof of any one or both of embodiments 16 and 19, wherein the monocotyledonous plant is selected from the group consisting of: corn, rice, wheat, oat, barley and millet.

21. The transgenic plant or tissues thereof of any one or more of embodiments 16, and 19-20, wherein the tissue of the monocotyledonous is grain.

22. The transgenic plant or tissues thereof of any one or more of embodiments 16, and 19-21, wherein the at least one animal-derived protein is expressed at a level in a range from 0.01 mg to 24.0 mg of the animal-derived protein per gram of grain.

23. The transgenic plant or tissues thereof of any one or more of embodiments 16, and 19-22, wherein the monocotyledonous plant is corn, and the tissue thereof is a corn grain.

24. The transgenic plant or tissues thereof of any one or more of embodiments 16, and 19-23, wherein the at least one animal-derived protein is expressed in the corn grain at a level in the range from 0.01 mg to 24.0 mg of the animal-derived protein per gram of corn grain.

25. The transgenic plant or tissues thereof of any one or more of embodiments 16, and 19-24, wherein the at least one animal-derived protein is expressed in an endosperm or aleurone cells of the corn grain.

26. The transgenic plant or tissues thereof of any one or more of embodiments 16, and 19-25, wherein the at least one animal-derived protein is expressed in the endosperm or aleurone cells of the corn grain at a level in the range from 0.01 mg to 24.0 mg of the animal-derived protein per gram of corn grain.

27. The transgenic plant or tissues thereof of any one or more of embodiments 16, and 19-24, wherein the at least one animal-derived protein is expressed in an embryo of the corn grain.

28. The transgenic plant or tissues thereof of any one or more of embodiments 16, 9-24, and 27, wherein the at least one animal-derived protein is expressed in the embryo of the corn grain at a level in the range from 0.01 mg to 24.0 mg of the animal-derived protein per gram of grain.

29. The transgenic plant or tissues thereof of any one or more of embodiments 16-28, wherein the synthetic polynucleotide comprises a nucleic acid encoding a myoglobin protein.

30. The transgenic plant or tissues thereof of any one or more of embodiments 16-29, wherein the nucleic acid encoding the myoglobin protein comprises a sequence with at least 90% identity to a reference sequence of SEQ ID NO: 32 or 78.

31. The transgenic plant or tissues thereof of any one or more of embodiments 16-30, wherein the myoglobin protein comprises an amino acid sequence with at least 95% identity to a reference sequence selected from the group consisting of: SEQ ID NOS: 1-3, 20, 40 and 58.

32. The transgenic plant or tissues thereof of any one or more of embodiments 16-28, wherein the synthetic polynucleotide comprises a nucleic acid encoding a hemoglobin protein.

33. The transgenic plant or tissues thereof of any one or more of embodiments 16-28 and 32, wherein the nucleic acid encoding the hemoglobin protein comprises a sequence with at least 90% identity to a reference sequence of SEQ ID NO: 33.

34. The transgenic plant or tissues thereof of any one or more of embodiments 16-28, and 32-33, wherein the hemoglobin protein comprises an amino acid sequence with at least 95% identity to a reference sequence selected from the group consisting of: SEQ ID NOS: 4-6, 21, and 41.

35. The transgenic plant or tissues thereof any one or more of embodiments 16-28, wherein the synthetic polynucleotide comprises a nucleic acid encoding an actin protein.

36. The transgenic plant or tissues thereof of any one or more of embodiments 16-28, and 35, wherein the synthetic nucleic acid encoding the actin protein comprises a sequence with at least 90% identity to a reference sequence of SEQ ID NO: 34.

37. The transgenic plant or tissues thereof of any one or more of embodiments 16-28, and 35-36, wherein the actin protein comprises an amino acid sequence with at least 95% identity to a reference sequence selected from the group consisting of: SEQ ID NOS: 7-9, 22, and 42.

38. The transgenic plant or tissues thereof of any one or more of embodiments 16-28, wherein the synthetic polynucleotide comprises a nucleic acid encoding a chymosin protein.

39. The transgenic plant or tissues thereof of any one or more of embodiments 16-28, and 38, wherein the nucleic acid encoding the chymosin protein comprises a sequence with at least 90% identity to a reference sequence of SEQ ID NO: 31.

40. The transgenic plant or tissues thereof of any one or more of embodiments 16-28, and 38-39, wherein the chymosin protein comprises an amino acid sequence with at least 95% identity to a reference sequence selected from the group consisting of: SEQ ID NOS: 14, 19, and 39.

41. The transgenic plant or tissues thereof of any one or more of embodiments 16-28, wherein the synthetic polynucleotide comprises a nucleic acid encoding a casein protein.

42. The transgenic plant or tissues thereof of any one or more of embodiments 16-28, and 41, wherein the nucleic acid encoding the casein protein comprises a sequence with at least 90% identity to a reference sequence selected from the group consisting of SEQ ID NOS: 27-30.

43. The transgenic plant or tissues thereof of any one or more of embodiments 16-28, and 41-43, wherein the casein protein comprises an amino acid sequence with at least 95% identity to a reference sequence selected from the group consisting of: SEQ ID NOS: 10-13, 15-18, and 35-38.

44. The transgenic plant or tissues thereof of any one or more of embodiments 16-28, wherein the synthetic polynucleotide comprises a nucleic acid encoding a glutamyl-tRNA reductase.

45. The transgenic plant or tissues thereof of any one or more of embodiments 16-28, and 44, wherein the nucleic acid encoding the glutamyl-tRNA reductase comprises a sequence with at least 90% identity to a reference sequence of SEQ ID NO: 79.

46. The transgenic plant or tissues thereof of any one or more of embodiments 16-28, and 44-45, wherein the glutamyl-tRNA reductase comprises an amino acid sequence with at least 90% identity to a reference sequence of SEQ ID NO: 59.

47. The transgenic plant or tissues thereof of any one or more of embodiments 16-28, wherein the synthetic polynucleotide comprises a nucleic acid encoding a ferrochelatase.

48. The transgenic plant or tissues thereof of any one or more of embodiments 16-28, and 47, wherein the nucleic acid encoding the ferrochelatase comprises a sequence with at least 90% identity to a reference sequence of SEQ ID NO: 81.

49. The transgenic plant or tissues thereof of any one or more of embodiments 16-28, and 47-48, wherein the ferrochelatase comprises an amino acid sequence with at least 90% identity to a reference sequence of: SEQ ID NO: 61.

50. An expression cassette comprising a synthetic nucleic acid encoding at least one animal-derived protein selected from the group consisting of a myoglobin, actin, hemoglobin, chymosin, and casein proteins.

51. The expression cassette of embodiment 50, further comprising at least one regulatory element selected from the group consisting of a promoter, signal peptide and terminator.

52. The expression cassette of any one or both of embodiments 50 and 51, wherein the synthetic nucleic acid encodes the myoglobin protein and comprises a sequence with at least 90% identity to the reference sequence of SEQ ID NO: 32, or 78.

53. The expression cassette of any one or more of embodiments 50-52, wherein the expression cassette comprises a synthetic nucleic acid that encodes the myoglobin protein and comprises a sequence with at least 90% identity to a reference sequence selected from the group consisting of: SEQ ID NOs: 48, 51-52, 64-66, 70, 72, and 74-77.

54. The expression cassette of any one or more of embodiments 50-53, wherein the expression cassette comprises a synthetic nucleic acid that encodes the glutamyl-tRNA reductase and comprises a sequence with at least 90% identity to a reference sequence of: SEQ ID NO: 67, 68 or 79.

55. The expression cassette of any one or more of embodiments 50-54, wherein the expression cassette comprises a synthetic nucleic acid that encodes the ferrochelatase and comprises a sequence with at least 90% identity to a reference sequence of: SEQ ID NO: 68, 69 or 81.

56. The expression cassette of any one or both of embodiments 50 and 51, wherein the synthetic nucleic acid encodes the hemoglobin protein and comprises a sequence with at least 90% identity to a reference sequence of SEQ ID NO: 33.

57. The expression cassette of any one or more of embodiments 50-51, and 56, wherein the expression cassette comprises the synthetic nucleic acid that encodes the hemoglobin protein and comprises a sequence with at least 90% identity to the sequence of SEQ ID NO: 49.

58. The expression cassette of any one or both of embodiments 50 and 51, wherein the synthetic nucleic acid encodes the actin protein and comprising a sequence with at least 90% identity to a reference sequence of SEQ ID NO: 34.

59. The expression cassette of any one or more of embodiments 50-51, and 58, wherein the expression cassette comprises the synthetic nucleic acid that encodes at the actin protein and comprises a sequence with at least 90% identity to the sequence of SEQ ID NO: 50.

60. The expression cassette of any one or both of embodiments 50 and 51, wherein the synthetic nucleic acid encodes the chymosin protein and comprises a sequence with at least 90% identity to the reference sequence of SEQ ID NO: 31.

61. The expression cassette of any one or more of embodiments 50-51, and 60, wherein the expression cassette comprises the synthetic nucleic acid that encodes the chymosin protein and comprises a sequence with at least 90% identity to the sequence of SEQ ID NO: 43.

62. The expression cassette of any one or both of embodiments 50 and 51, wherein the synthetic nucleic acid encodes the casein protein, and comprises a sequence with at least 90% identity to a reference sequence selected from the group consisting of SEQ ID NOS: 27-30.

63. The expression cassette of any one or more of embodiments 50-51, and 62 wherein the expression cassette comprises the synthetic nucleic acid that encodes the casein protein and comprises a sequence with at least 90% identity to a reference sequence selected from the group consisting of: SEQ ID NO: 44-47.

64. A plant-based meat composition comprising at least one of the transgenic plants or tissues thereof of any one or more of embodiments 1-49.

65. A plant-based meat composition comprising any one of the animal-derived proteins isolated from transgenic plants or tissues thereof of any one or more of embodiments 1-49.

66. The plant-based meat composition of embodiment 65, wherein the animal derived protein comprises the myoglobin protein encoded by the synthetic nucleic acid sequence with at least 90% identity to the reference sequence selected from the group consisting of SEQ ID NO: 32.

67. The plant-based meat composition of any one or more of embodiments 65-66, wherein the animal-derived protein comprises the hemoglobin encoded by the synthetic nucleic acid sequence with at least 90% identity to the reference sequence of SEQ ID NO: 33.

68. The plant-based meat composition of any one or more of embodiments 65-67, wherein the animal-derived protein comprises the actin encoded by the synthetic nucleic acid sequence with at least 90% identity to the reference sequence of SEQ ID NO: 34.

69. The plant-based meat composition of any one or more of embodiments 65-68, wherein the animal-derived protein comprises a combination of myoglobin, hemoglobin and actin proteins.

70. A plant-based cheese composition comprising at least one of any of the transgenic plants or tissues thereof of any one or more of embodiments 1-49.

71. A plant-based cheese composition comprising an animal-based protein isolated from transgenic plants or tissues thereof of any one or more of embodiments 1-49.

72. The plant-based cheese composition of embodiment 71, wherein the animal-derived protein comprises the chymosin encoded by the synthetic nucleic acid sequence with at least 90% identity to the reference sequence of SEQ ID NO: 31.

73. The plant-based cheese composition of any one or both of embodiments 71 and 72, wherein the animal-derived protein comprises the casein protein encoded by the synthetic nucleic acid sequence with at least 90% identity to the reference sequence selected from the group consisting of: SEQ ID NOs: 27-30.

74. The plant-based cheese composition of any one or more of embodiments 71-73, wherein the animal-derived protein comprises a combination of the chymosin protein and at least one casein proteins.

75. A process for isolating at least one animal-derived protein from a transgenic plant or tissues thereof of any one or more of embodiments 1-49.

76. The process of embodiment 75, wherein the isolation is a modified wet milling or dry milling process.

77. The process of any one or both of embodiments 75 and 76, wherein the transgenic plant is a dicotyledonous plant.

78. The process of any one or more of embodiments 75-76, wherein the dicotyledonous plant is selected from the group consisting of soybeans, legumes, such as pea, beans, lentils, and peanuts, sugar beet, cabbage, potato, and tomato.

79. The process of any one or both of embodiments 75 and 76, wherein the transgenic plant is a monocotyledonous plant.

80. The process of any one or more of embodiments 75, 76, and 79, wherein the monocotyledonous plant is corn, and the tissue is a corn grain.

81. The process of any one or more of embodiments 75-80, wherein the process is the modified wet milling.

82. The process of any one or more of embodiments 75-81, wherein the isolation is performed by using an organic solvent extraction.

83. The process of any one or more of embodiments 75-81, wherein the isolation is performed by using an aqueous extraction.

84. The process of any one or more of embodiments 75, 76, and 80-83, wherein the at least one animal-derived protein is isolated from an embryo of the corn grain.

85. The process of any one or more of embodiments 75, 76, and 80-83, wherein the at least one animal-derived protein is isolated from an endosperm of the corn grain.

86. The process of any one or more of embodiments 75, 76, and 80-85, wherein prior to isolation the process comprises the step of deembryonization or degermination of the corn grain.

87. The process of any one or more of embodiments 75, 76, and 80-86, wherein upon the deembryonization or degermination, the isolation comprises extraction of the animal-derived protein from the remaining fiber and endosperm.

88. The process of any one or more of embodiments 75, 76, and 80-87, wherein prior to isolation the process comprises steeping of the corn grain in a steep water.

89. The process of any one or more of embodiments 75, 76, and 80-88, wherein the at least one animal-derived protein is removed from the corn grain in the steep water.

90. The process of any one or more of embodiments 75, 76, and 80-89, wherein the isolation occurs by extraction of the at least one animal-derived protein from the corn gluten feed, or from the corn gluten meal.

91. The process of any one or more of embodiments 75-90, wherein the animal-derived protein is further purified using heating of the extract, centrifugation, evaporation, filtration, pressed to remove liquid, or drying.

92. The process of any one or more of embodiments 75-91, wherein the aqueous extraction is performed by using an aqueous buffer.

93. The process of any one or more of embodiments 75-92, wherein the aqueous buffer comprises a detergent.

94. The process of any one or more of embodiments 75-93, wherein the aqueous buffer is selected from the group consisting of: Britton-Robinson (BR) polybuffer, pH4 to pH9; deionized water; 100 mM Tris, 10 mM EDTA; 100 mM sodium phosphate, pH6.5; 30 mM sodium carbonate/bicarbonate, pH10.8. M.

95. The process of any one or more of embodiments 75-94, wherein the aqueous buffer comprises a detergent selected from the group consisting of: Sarkosyl, Tween, or Sodium Dodecyl Sulfate.

96. The process of any one or more of embodiments 75-95, wherein the concentration of the detergent is less than 3% (v/v) of the detergent per buffer.

Further embodiments herein may be formed by supplementing an embodiment with one or more elements from any one or more other embodiments herein, and/or substituting one or more elements from one embodiment with one or more elements from one or more other embodiments.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1. Descriptions of Sequences and Constructs for Expressing Casein, Chymosin, Myoglobin, Hemoglobin and Actin in Maize

Casein, chymosin, myoglobin, hemoglobin, and actin are normally produced by animals, where they play important biological roles. Engineering plants to make these molecules provides a novel source that does not require the native animal host and enables novel processes for obtaining these molecules to be used as very specific, plant-derived (or, equivalently, non-animal derived) new food ingredients. These processes would not be otherwise possible without the engineered plant as a raw material, and isolation of the recombinant casein, chymosin, myoglobin, hemoglobin, and actin from plants enables their use in new food compositions that can be completely plant derived and free of any animal derived components.

Over-expression of casein, chymosin, myoglobin, hemoglobin, or actin in plants can be used to increase the value of the resulting grain, and protein by-products derived from grain processing, when used as a raw material in modified wet-milling, dry milling, or dry-grind processes that are designed to isolate the recombinant protein. Expressing casein, chymosin, myoglobin, hemoglobin and actin in plants presents fewer risks (e.g., adventitious presence of viral pathogens) to the food chain than sourcing them from animals who may harbor zoonotic pathogens; further, it also presents fewer risks than microbial or mammalian-based production, which can propagate contaminating viral or bacterial pathogens in a culture. Plant expression is considered more safe as plants do not harbor human pathogens, and those that may grow on the plant are easily removed by washing of the plant material and processing. Since the target proteins (casein, chymosin, myoglobin, hemoglobin, and actin) are already present in the food chain and there is a long history of their tolerance and safe use, as well as that of the plant production host, plant production represents a significant reduction in production risks for these food ingredients.

The bovine, chicken and swine protein sequences for myoglobin, hemoglobin and actin were retrieved from the UniProt database of protein sequences (https://www.uniprot.org/) and shown in Table 1. Multiple sequence alignments of these proteins were performed with the Clustal Omega software (version 1.2.4) that is available online (https://www.ebi.ac.uk/Tools/msa/clustalo/). Bovine myoglobin (P02192) was used for developing expression vectors (described below) and the sequence alignments with chicken (P02197) and swine (P02189) homologs are shown below.

| | |
|---|---|
| P02197 | MGLSDQEWQQVLTIWGKVEADIAGHGHEVLMRLFHDHPETLDREDKFKGLKTPDQMKGSE 60 |
| P02192 | MGLSDGEWQLVLNAWGKVEADVAGHGQEVLIRLFTGHPETLEKEDKFKHLKTEAEMKASE 60 |
| P02189 | MGLSDGEWQLVLNVWGKVEADVAGHGQEVLIRLFKGHPETLEKEDKFKHLKSEDEMKASE 60 |
| | ***** *** **. *******:****:***:*** .*****::***** **: :**.** |
| P02197 | DLKKHGATVLTQLGKILKQKGNHESELKPLAQTHATKHKIPVKYLEFISEVIIKVIAEKH 120 |
| P02192 | DLKKHGNTVLTALGGILKKKGHHEAEVKHLAESHANKHKIPVKYLEFISDAIIHVLHAKH 120 |
| P02189 | DLKKHGNTVLTALGGILKKKGHHEAELTPLAQSHATKHKIPVKYLEFISEAIIQVLQSKH 120 |
| | ****** **** ** ***:**:**:*:. **::**.*************:.**:*: ** |
| P02197 | AADFGADSQAAMKKALELFRNDMASKYKEFGFQG 154 [SEQ ID NO: 2] |
| P02192 | PSDFGADAQAAMSKALELFRNDMAAQYKVLGFHG 154 [SEQ ID NO: 1] |
| P02189 | PGDFGADAQGAMSKALELFRNDMAAKYKELGFQG 154 [SEQ ID NO: 3] |
| | .******:*.**.***********::** :**:* |

| Percent Identity Matrix: | | | |
|---|---|---|---|
| 1: P02197 | 100.00 | 72.73 | 76.62 |
| 2: P02192 | 72.73 | 100.00 | 88.31 |
| 3: P02189 | 76.62 | 88.31 | 100.00 |

The alpha subunit of bovine hemoglobin (P01966) was used for developing expression vectors (described below) and the sequence alignments with chicken (P01994) and swine (P01965) homologs are shown below.

| | |
|---|---|
| P01994 | MVLSAADKNNVKGIFTKIAGHAEEYGAETLERMFTTYPPTKTYFPHFDLSHGSAQIKGHG 60 |
| P01966 | MVLSAADKGNVKAAWGKVGGHAAEYGAEALERMFLSFPTTKTYFPHFDLSHGSAQVKGHG 60 |
| P01965 | MVLSAADKANVKAAWGKVGGQAGAHGAEALERMFLGFPTTKTYFPHFNLSHGSDQVKAHG 60 |
| | ******** ***. : *:.*:* :***:***** :* ********:***** *:*.** |
| P01994 | KKVVAALIEAANHIDDIAGTLSKLSDLHAHKLRVDPVNFKLLGQCFLVVVAIHHPAALTP 120 |
| P01966 | AKVAAALTKAVEHLDDLPGALSELSDLHAHKLRVDPVNFKLLSHSLLVTLASHLPSDFTP 120 |
| P01965 | QKVADALTKAVGHLDDLPGALSALSDLHAHKLRVDPVNFKLLSHCLLVTLAAHHPDDENP 120 |
| | **. ***:*. *:**: * ** *******************.:.:**.:* * * .:* |
| P01994 | EVHASLDKFLCAVGTVLTAKYR 142 [SEQ ID NO: 5] |
| P01966 | AVHASLDKFLANVSTVLTSKYR 142 [SEQ ID NO: 4] |
| P01965 | SVHASLDKFLANVSTVLTSKYR 142 [SEQ ID NO: 6] |
| | ********************** |

| Percent Identity Matrix: | | | |
|---|---|---|---|
| 1: P01994 | 100.00 | 71.13 | 66.90 |
| 2: P01966 | 71.13 | 100.00 | 86.62 |
| 3: P01965 | 66.90 | 86.62 | 100.00 |

Similarly, the alpha subunit of bovine (P68138) actin was used for developing expression vectors (described below) and the sequence alignments with chicken (P68139) and swine (P68137) homologs are shown below.

| | |
|---|---|
| P68138 | MCDEDETTALVCDNGSGLVKAGFAGDDAPRAVFPSIVGRPRHQGVMVGMGQKDSYVGDEA 60 |
| P68139 | MCDEDETTALVCDNGSGLVKAGFAGDDAPRAVFPSIVGRPRHQGVMVGMGQKDSYVGDEA 60 |
| P68137 | MCDEDETTALVCDNGSGLVKAGFAGDDAPRAVFPSIVGRPRHQGVMVGMGQKDSYVGDEA 60 |
| | ************************************************************ |
| P68138 | QSKRGILTLKYPIEHGIITNWDDMEKIWHHTFYNELRVAPEEHPTLLTEAPLNPKANREK 120 |
| P68139 | QSKRGILTLKYPIEHGIITNWDDMEKIWHHTFYNELRVAPEEHPTLLTEAPLNPKANREK 120 |
| P68137 | QSKRGILTLKYPIEHGIITNWDDMEKIWHHTFYNELRVAPEEHPTLLTEAPLNPKANREK 120 |
| | ************************************************************ |
| P68138 | MTQIMFETFNVPAMYVAIQAVLSLYASGRTTGIVLDSGDGVTHNVPIYEGYALPHAIMRL 180 |
| P68139 | MTQIMFETFNVPAMYVAIQAVLSLYASGRTTGIVLDSGDGVTHNVPIYEGYALPHAIMRL 180 |
| P68137 | MTQIMFETFNVPAMYVAIQAVLSLYASGRTTGIVLDSGDGVTHNVPIYEGYALPHAIMRL 180 |
| | ************************************************************ |
| P68138 | DLAGRDLTDYLMKILTERGYSFVTTAEREIVRDIKEKLCYVALDFENEMATAASSSSLEK 240 |
| P68139 | DLAGRDLTDYLMKILTERGYSFVTTAEREIVRDIKEKLCYVALDFENEMATAASSSSLEK 240 |
| P68137 | DLAGRDLTDYLMKILTERGYSFVTTAEREIVRDIKEKLCYVALDFENEMATAASSSSLEK 240 |
| | ************************************************************ |
| P68138 | SYELPDGQVITIGNERFRCPETLFQPSFIGMESAGIHETTYNSIMKCDIDIRKDLYANNV 300 |
| P68139 | SYELPDGQVITIGNERFRCPETLFQPSFIGMESAGIHETTYNSIMKCDIDIRKDLYANNV 300 |
| P68137 | SYELPDGQVITIGNERFRCPETLFQPSFIGMESAGIHETTYNSIMKCDIDIRKDLYANNV 300 |
| | ************************************************************ |
| P68138 | MSGGTTMYPGIADRMQKEITALAPSTMKIKIIAPPERKYSVWIGGSILASLSTFQQMWIT 360 |
| P68139 | MSGGTTMYPGIADRMQKEITALAPSTMKIKIIAPPERKYSVWIGGSILASLSTFQQMWIT 360 |
| P68137 | MSGGTTMYPGIADRMQKEITALAPSTMKIKIIAPPERKYSVWIGGSILASLSTFQQMWIT 360 |
| | ************************************************************ |
| P68138 | KQEYDEAGPSIVHRKCF 377 [SEQ ID NO: 7] |
| P68139 | KQEYDEAGPSIVHRKCF 377 [SEQ ID NO: 8] |
| P68137 | KQEYDEAGPSIVHRKCF 377 [SEQ ID NO: 9] |
| | ***************** |

| Percent Identity Matrix: | | | |
|---|---|---|---|
| 1: P68138 | 100.00 | 100.00 | 100.00 |
| 2: P68139 | 100.00 | 100.00 | 100.00 |
| 3: P68137 | 100.00 | 100.00 | 100.00 |

Bovine (Bos Taurus) protein sequences of casein (alphaS1-, alphaS2-, beta-, and kappa-subunits), chymosin B, myoglobin (MYG; bovine (Bos taurus) myoglobin is referred to as btMYG), hemoglobin (alpha subunit, HBA; bovine (Bos taurus) hemoglobin is designated as btHBA), and actin (alpha subunit) were retrieved from protein databases available either at the National Center for Biotechnology Information (NCBI) (https://www.nbci.nlm.nih.gov/) or at the UniProt Knowledgebase (UniProtKB) (https://www.uniprot.org/). These original bovine protein sequences, where underlined sequences at their N-terminal ends denote signal peptide, are listed in Table 1:

TABLE 1
Native amino acid sequences of food-related proteins
identified in public databases.

| SEQ | | Accession | |
|---|---|---|---|
| ID | | Number | |
| NO | Protein name | | Amino acid sequence |
| 1 | Bovine myoglobin | P02192 | MGLSDGEWQLVLNAWGKVEADVAGHGQEVLIR |
| | | | LFTGHPETLEKFDKFKHLKTEAEMKASEDLKKH |
| | | | GNTVLTALGGILKKKGHHEAEVKHLAESHANKH |
| | | | KIPVKYLEFISDAIIHVLHAKHPSDFGADAQAAMS |
| | | | KALELFRNDMAAQYKVLGFHG |
| 2 | Chicken | P02197 | MGLSDQEWQQVLTIWGKVEADIAGHGHEVLMR |
| | myoglobin | | LFHDHPETLDRFDKFKGLKTPDQMKGSEDLKKH |
| | | | GATVLTQLGKILKQKGNHESELKPLAQTHATKH |
| | | | KIPVKYLEFISEVIIKVIAEKHAADFGADSQAAMK |
| | | | KALELFRNDMASKYKEFGFQG |
| 3 | Swine myoglobin | P02189 | MGLSDGEWQLVLNVWGKVEADVAGHGQEVLIR |
| | | | LFKGHPETLEKFDKFKHLKSEDEMKASEDLKKH |
| | | | GNTVLTALGGILKKKGHHEAELTPLAQSHATKH |
| | | | KIPVKYLEFISEAIIQVLQSKHPGDFGADAQGAM |
| | | | SKALELFRNDMAAKYKELGFQG |
| 4 | Bovine alpha | P01966 | MVLSAADKGNVKAAWGKVGGHAAEYGAEALE |
| | hemoglobin | | RMFLSFPTTKTYFPHFDLSHGSAQVKGHGAKVA |
| | | | AALTKAVEHLDDLPGALSELSDLHAHKLRVDPV |
| | | | NFKLLSHSLLVTLASHLPSDFTPAVHASLDKFLA |
| | | | NVSTVLTSKYR |
| 5 | Chicken | P01994 | MVLSAADKNNVKGIFTKIAGHAEEYGAETLERM |
| | hemoglobin | | FTTYPPTKTYFPHFDLSHGSAQIKGHGKKVVAA |
| | | | LIEAANHIDDIAGTLSKLSDLHAHKLRVDPVNFKL |
| | | | LGQCFLVVVAIHHPAALTPEVHASLDKFLCAVGT |
| | | | VLTAKYR |
| 6 | Swine hemoglobin | P01965 | MVLSAADKANVKAAWGKVGGQAGAHGAEALE |
| | | | RMFLGFPTTKTYFPHFNLSHGSDQVKAHGQKV |
| | | | ADALTKAVGHLDDLPGALSALSDLHAHKLRVDP |
| | | | VNFKLLSHCLLVTLAAHHPDDFNPSVHASLDKFL |
| | | | ANVSTVLTSKYR |
| 7 | Bovine alpha actin | P68138 | MCDEDETTALVCDNGSGLVKAGFAGDDAPRAV |
| | | | FPSIVGRPRHQGVMVGMGQKDSYVGDEAQSK |
| | | | RGILTLKYPIEHGIITNWDDMEKIWHHTFYNELRV |
| | | | APEEHPTLLTEAPLNPKANREKMTQIMFETFNV |
| | | | PAMYVAIQAVLSLYASGRTTGIVLDSGDGVTHN |
| | | | VPIYEGYALPHAIMRLDLAGRDLTDYLMKILTER |
| | | | GYSFVTTAEREIVRDIKEKLCYVALDFENEMATA |
| | | | ASSSSLEKSYELPDGQVITIGNERFRCPETLFQP |
| | | | SFIGMESAGIHETTYNSIMKCDIDIRKDLYANNV |
| | | | MSGGTTMYPGIADRMQKEITALAPSTMKIKIIAPP |
| | | | ERKYSVWIGGSILASLSTFQQMWITKQEYDEAG |
| | | | PSIVHRKCF |
| 8 | Chicken actin | P68139 | MCDEDETTALVCDNGSGLVKAGFAGDDAPRAV |
| | | | FPSIVGRPRHQGVMVGMGQKDSYVGDEAQSK |
| | | | RGILTLKYPIEHGIITNWDDMEKIWHHTFYNELRV |
| | | | APEEHPTLLTEAPLNPKANREKMTQIMFETFNV |
| | | | PAMYVAIQAVLSLYASGRTTGIVLDSGDGVTHN |
| | | | VPIYEGYALPHAIMRLDLAGRDLTDYLMKILTER |
| | | | GYSFVTTAEREIVRDIKEKLCYVALDFENEMATA |
| | | | ASSSSLEKSYELPDGQVITIGNERFRCPETLFQP |
| | | | SFIGMESAGIHETTYNSIMKCDIDIRKDLYANNV |
| | | | MSGGTTMYPGIADRMQKEITALAPSTMKIKIIAPP |
| | | | ERKYSVWIGGSILASLSTFQQMWITKQEYDEAG |
| | | | PSIVHRKCF |
| 9 | Swine actin | P68137 | MCDEDETTALVCDNGSGLVKAGFAGDDAPRAV |
| | | | FPSIVGRPRHQGVMVGMGQKDSYVGDEAQSK |
| | | | RGILTLKYPIEHGIITNWDDMEKIWHHTFYNELRV |
| | | | APEEHPTLLTEAPLNPKANREKMTQIMFETFNV |
| | | | PAMYVAIQAVLSLYASGRTTGIVLDSGDGVTHN |
| | | | VPIYEGYALPHAIMRLDLAGRDLTDYLMKILTER |
| | | | GYSFVTTAEREIVRDIKEKLCYVALDFENEMATA |
| | | | ASSSSLEKSYELPDGQVITIGNERFRCPETLFQP |
| | | | SFIGMESAGIHETTYNSIMKCDIDIRKDLYANNV |
| | | | MSGGTTMYPGIADRMQKEITALAPSTMKIKIIAPP |
| | | | ERKYSVWIGGSILASLSTFQQMWITKQEYDEAG |
| | | | PSIVHRKCF |
| 10 | Bovine alpha- | NP_851372.1 | MKLLILTCLVAVALARPKHPIKHQGLPQEVLNEN |
| | S1-casein | | LLRFFVAPFPEVFGKEKVNELSKDIGSESTEDQA |
| | | | MEDIKQMEAESISSSEEIVPNSVEQKHIQKEDVP |
| | | | SERYLGYLEQLLRLKKYKVPQLEIVPNSAEERLH |
| | | | SMKEGIHAQQKEPMIGVNQELAYFYPELFRQFY |
| | | | QLDAYPSGAWYYVPLGTQYTDAPSFSDIPNPIG |
| | | | SENSEKTTMPLW |
| 11 | Bovine alpha- | NP_776953.1 | MKFFIFTCLLAVALAKNTMEHVSSSEESIISQETY |
| | S2-casein | | KQEKNMAINPSKENLCSTFCKEVVRNANEEEYS |
| | | | IGSSSEESAEVATEEVKITVDDKHYQKALNEINQ |
| | | | FYQKFPQYLQYLYQGPIVLNPWDQVKRNAVPIT |
| | | | PTLNREQLSTSEENSKKTVDMESTEVFTKKTKL |
| | | | TEEEKNRLNFLKKISQRYQKFALPQYLKTVYQH |
| | | | QKAMKPWIQPKTKVIPYVRYL |
| 12 | Bovine beta- | AAA30480.1 | MKVLILACLVALALARELEELNVPGEIVESLSSSE |
| | casein | | ESITRINKKIEKFQSEEQQQTEDELQDKIHPFAQ |
| | | | TQSLVYPFPGPIPNSLPQNIPPLTQTPVVVPPFL |
| | | | QPEVMGVSKVKEAMAPKHKEMPFPKYPVEPFT |
| | | | ESQSLTLTDVENLHLPLPLLQSWMHQPHQPLPP |
| | | | TVMFPPQSVLSLSQSKVLPVPQKAVPYPQRDM |
| | | | PIQAFLLYQEPVLGPVRGPFPIIV |
| 13 | Bovine kappa- | NP 776719.1 | MMKSFFLVVTILALTLPFLGAQEQNQEQPIRCEK |
| | casein | | DERFFSDKIAKYIPIQYVLSRYPSYGLNYYQQKP |
| | | | VALINNQFLPYPYYAKPAAVRSPAQILQWQVLS |
| | | | NTVPAKSCQAQPTTMARHPHPHLSFMAIPPKKN |
| | | | QDKTEIPTINTIASGEPTSTPTTEAVESTVATLED |
| | | | SPEVIESPPEINTVQVTSTAV |
| 14 | Bovine | P00794 | MRCLVVLLAVFALSQGAEITRIPLYKGKSLRKAL |
| | chymosin | | KEHGLLEDFLQKQQYGISSKYSGFGEVASVPLT |
| | B | | NYLDSQYFGKIYLGTPPQEFTVLFDTGSSDFWV |
| | | | PSIYCKSNACKNHQRFDPRKSSTFQNLGKPLSI |
| | | | HYGTGSMQGILGYDTVTVSNIVDIQQTVGLSTQ |
| | | | EPGDVFTYAEFDGILGMAYPSLASEYSIPVFDN |
| | | | MMNRHLVAQDLFSVYMDRNGQESMLTLGAIDP |
| | | | SYYTGSLHWVPVTVQQYWQFTVDSVTISGVVV |
| | | | ACEGGCQAILDTGTSKLVGPSSGILNIQQAIGAT |
| | | | QNQYGEFDIDCDNLSYMPTVVFEINGKMYPLTP |
| | | | SAYTSQDQGFCTSGFQSENHSQKWILGDVFIRE |
| | | | YYSVFDRANNLVGLAKAI |

The N-terminal native signal sequences or the first amino acid methionine (M) were removed and resulting amino acid sequences (SEQ ID NOS: 15-22) are listed in Table 2. These protein sequences were used in proteins expressed in plants as described below.

TABLE 2
Amino acid sequences of food-related bovine proteins
expressed in maize

| SEQ | Bovine | | |
|---|---|---|---|
| ID | truncated | Accession | |
| NO | protein name | Number | Amino acid sequence |
| 15 | Alpha-S1-casein | NP_851372.1 | RPKHPIKHQGLPQEVLNENLLRFFVAPFPEVFG |
| | (CasA1; trunc) | | KEKVNELSKDIGSESTEDQAMEDIKQMEAESISS |
| | | | SEEIVPNSVEQKHIQKEDVPSERYLGYLEQLLRL |
| | | | KKYKVPQLEIVPNSAEERLHSMKEGIHAQQKEP |
| | | | MIGVNQELAYFYPELFRQFYQLDAYPSGAWYYV |
| | | | PLGTQYTDAPSFSDIPNPIGSENSEKTTMPLW |
| 16 | Alpha-S2-casein | NP_776953.1 | KNTMEHVSSSEESIISQETYKQEKNMAINPSKEN |
| | (CasA2; trunc) | | LCSTFCKEVVRNANEEEYSIGSSSEESAEVATE |
| | | | EVKITVDDKHYQKALNEINQFYQKFPQYLQYLY |
| | | | QGPIVLNPWDQVKRNAVPITPTLNREQLSTSEE |
| | | | NSKKTVDMESTEVFTKKTKLTEEEKNRLNFLKKI |
| | | | SQRYQKFALPQYLKTVYQHQKAMKPWIQPKTK |
| | | | VIPYVRYL |
| 17 | Beta-casein | AAA30480.1 | RELEELNVPGEIVESLSSSEESITRINKKIEKFQS |
| | (CasB; trunc) | | EEQQQTEDELQDKIHPFAQTQSLVYPFPGPIPN |
| | | | SLPQNIPPLTQTPVVVPPFLQPEVMGVSKVKEA |
| | | | MAPKHKEMPFPKYPVEPFTESQSLTLTDVENLH |
| | | | LPLPLLQSWMHQPHQPLPPTVMFPPQSVLSLS |
| | | | QSKVLPVPQKAVPYPQRDMPIQAFLLYQEPVLG |
| | | | PVRGPFPIIV |
| 18 | Kappa-casein | NP_776719.1 | QEQNQEQPIRCEKDERFFSDKIAKYIPIQYVLSR |
| | (CasK; trunc) | | YPSYGLNYYQQKPVALINNQFLPYPYYAKPAAV |
| | | | RSPAQILQWQVLSNTVPAKSCQAQPTTMARHP |
| | | | HPHLSFMAIPPKKNQDKTEIPTINTIASGEPTSTP |
| | | | TTEAVESTVATLEDSPEVIESPPEINTVQVTSTAV |
| 19 | Chymosin B | P00794 | AEITRIPLYKGKSLRKALKEHGLLEDFLQKQQYGI |
| | (CymB; trunc) | | SSKYSGFGEVASVPLTNYLDSQYFGKIYLGTPP |
| | | | QEFTVLFDTGSSDFWVPSIYCKSNACKNHQRFD |
| | | | PRKSSTFQNLGKPLSIHYGTGSMQGILGYDTVT |
| | | | VSNIVDIQQTVGLSTQEPGDVFTYAEFDGILGMA |
| | | | YPSLASEYSIPVFDNMMNRHLVAQDLFSVYMDR |
| | | | NGQESMLTLGAIDPSYYTGSLHWVPVTVQQYW |
| | | | QFTVDSVTISGVVVACEGGCQAILDTGTSKLVG |
| | | | PSSGILNIQQAIGATQNQYGEFDIDCDNLSYMPT |
| | | | WVFEINGKMYPLTPSAYTSQDQGFCTSGFQSEN |
| | | | HSQKWILGDVFIREYYSVFDRANNLVGLAKAI |
| 20 | Myoglobin (MYG; | P02192 | GLSDGEWQLVLNAWGKVEADVAGHGQEVLIRL |
| | trunc) | | FTGHPETLEKFDKFKHLKTEAEMKASEDLKKHG |
| | | | NTVLTALGGILKKKGHHEAEVKHLAESHANKHKI |
| | | | PVKYLEFISDAIIHVLHAKHPSDFGADAQAAMSK |
| | | | ALELFRNDMAAQYKVLGFHG |
| 21 | Alpha hemoglobin | P01966 | VLSAADKGNVKAAWGKVGGHAAEYGAEALER |
| | (HBA; trunc) | | MFLSFPTTKTYFPHFDLSHGSAQVKGHGAKVAA |
| | | | ALTKAVEHLDDLPGALSELSDLHAHKLRVDPVN |
| | | | FKLLSHSLLVTLASHLPSDFTPAVHASLDKFLAN |
| | | | VSTVLTSKYR |
| 22 | Alpha actin | P68138 | CDEDETTALVCDNGSGLVKAGFAGDDAPRAVF |
| | (ACTA; trunc) | | PSIVGRPRHQGVMVGMGQKDSYVGDEAQSKRG |
| | | | ILTLKYPIEHGIITNWDDMEKIWHHTFYNELRVA |
| | | | PEEHPTLLTEAPLNPKANREKMTQIMFETFNVP |
| | | | AMYVAIQAVLSLYASGRTTGIVLDSGDGVTHNV |
| | | | PIYEGYALPHAIMRLDLAGRDLTDYLMKILTERG |
| | | | YSFVTTAEREIVRDIKEKLCYVALDFENEMATAA |
| | | | SSSSLEKSYELPDGQVITIGNERFRCPETLFQPS |
| | | | FIGMESAGIHETTYNSIMKCDIDIRKDLYANNVM |
| | | | SGGTTMYPGIADRMQKEITALAPSTMKIKIIAPPE |
| | | | RKYSVWIGGSILASLSTFQQMWITKQEYDEAGP |
| | | | SIVHRKCF |

The protein sequences in Table 2 were subjected to maize DNA codon optimization using the GenSmart online tool that is available at the GenScript website (https://www.genescript.com/genesmart-free-gene-codon-optimization.html). This program creates a novel gene coding DNA sequence that expresses at a higher level than the native gene sequence in corn. Maize codon optimized nucleotide sequences encoding food-related bovine proteins are listed in Table 3.

TABLE 3
Maize codon optimized nucleotide sequences of food-related
bovine proteins

| SEQ | | |
|---|---|---|
| ID | | |
| NO | Gene name | Nucleotide sequence |
| 27 | Alpha-S1-casein | CGGCCCAAGCATCCTATAAAACATCAAGGTTTGCCTCAAGAA |
| | (CasA1; trunc) | GTTTTGAATGAAAATTTGCTCCGCTTCTTCGTCGCCCCCTTTC |
| | | CGGAGGTTTTTGGGAAAGAGAAGGTAAATGAGCTCAGCAAG |
| | | GACATTGGTTCTGAAAGTACAGAAGACCAGGCGATGGAGGAT |
| | | ATCAAGCAGATGGAAGCAGAGTCCATATCTTCTTCAGAGGAG |
| | | ATCGTCCCTAACAGCGTGGAGCAAAAGCACATCCAGAAGGA |
| | | GGACGTGCCGTCGGAGCGCTACCTGGGATATTTAGAGCAGC |
| | | TGCTCCGTCTCAAGAAGTACAAGGTGCCACAACTGGAAATTG |
| | | TTCCCAACAGTGCTGAAGAAAGACTTCACTCCATGAAAGAAG |
| | | GCATCCACGCGCAGCAGAAAGAACCAATGATTGGCGTCAAC |
| | | CAAGAGCTTGCCTACTTCTATCCTGAGCTGTTCAGGCAGTTC |
| | | TACCAGCTGGATGCTTATCCGTCCGGCGCGTGGTACTACGTG |
| | | CCGCTGGGCACGCAGTATACTGATGCACCATCATTTTCGGAC |
| | | ATCCCAAATCCAATTGGGAGCGAGAACTCCGAGAAAACCACC |
| | | ATGCCGCTGTGG |
| 28 | Alpha-S2-casein | AAAAACACAATGGAGCATGTTAGTTCTTCGGAGGAGAGCATA |
| | (CasA2; trunc) | ATTTCACAAGAAACATACAAGCAGGAGAAGAACATGGCCATC |
| | | AACCCGTCGAAAGAGAACCTCTGTAGTACTTTCTGCAAGGAA |
| | | GTTGTGAGGAATGCTAATGAAGAGGAGTACAGCATTGGTTCA |
| | | TCCTCTGAGGAAAGCGCGGAGGTTGCTACAGAAGAGGTCAA |
| | | GATCACTGTTGATGACAAGCACTACCAAAAGGCATTGAATGA |
| | | GATAAATCAGTTCTACCAAAAATTTCCGCAGTATTTGCAGTAC |
| | | CTGTATCAAGGCCCCATCGTGCTGAATCCGTGGGACCAGGT |
| | | GAAGCGCAACGCAGTGCCGATCACACCTACTCTTAACCGTGA |
| | | GCAGCTCTCCACGTCTGAAGAAAATAGCAAAAAAACAGTAGA |
| | | CATGGAATCAACTGAGGTCTTCACCAAAAAGACCAAGTTAAC |
| | | CGAGGAGGAAAAGAATAGACTGAACTTCCTCAAGAAAATTTC |
| | | CCAGAGGTATCAAAAGTTTGCGCTGCCACAATATCTTAAAAC |
| | | GGTGTACCAGCACCAGAAGGCCATGAAGCCCTGGATTCAGC |
| | | CAAAGACGAAGGTCATTCCTTATGTCCGCTACCTG |
| 29 | Beta-casein | AGAGAACTGGAGGAGCTGAATGTGCCCGGCGAGATTGTTGA |
| | (CasB; trunc) | GAGCTTGTCGTCGTCAGAAGAATCCATCACCCGGATCAACAA |
| | | GAAGATTGAGAAGTTCCAGTCTGAAGAGCAGCAGCAAACAGA |
| | | GGATGAGCTTCAGGACAAGATACATCCATTCGCCCAGACACA |
| | | ATCTCTCGTCTACCCGTTCCCAGGCCCCATCCCAAACAGTCT |
| | | TCCTCAAAATATTCCGCCGCTGACGCAGACCCCCGTGGTTGT |
| | | TCCTCCCTTCCTGCAGCCGGAGGTGATGGGCGTCAGCAAGG |
| | | TGAAAGAAGCGATGGCCCCCAAGCACAAGGAGATGCCGTTT |
| | | CCAAAATATCCAGTTGAACCTTTTACTGAATCTCAAAGTTTGA |
| | | CGTTAACTGATGTGGAGAACCTCCACCTCCCTCTTCCATTGC |
| | | TACAGTCATGGATGCATCAACCTCACCAGCCTTTGCCGCCGA |
| | | CCGTCATGTTCCCTCCACAGAGCGTGCTGTCCCTCTCCCAAT |
| | | CAAAAGTTCTACCCGTACCCCAGAAAGCTGTGCCATATCCTC |
| | | AGAGGGACATGCCAATACAAGCATTTCTGCTCTACCAGGAGC |
| | | CGGTACTTGGACCTGTCCGCGGGCCATTTCCTATCATTGTC |
| 30 | Kappa-casein | CAAGAACAAAACCAGGAGCAGCCGATACGTTGTGAGAAGGA |
| | (CasK; trunc) | CGAGAGGTTTTTTTCTGATAAAATTGCCAAGTACATCCCTATT |
| | Chymosin B | CAGTACGTTCTTTCGCGCTACCCGTCCTACGGGCTGAACTAC |
| | | TATCAGCAGAAGCCGGTTGCACTCATCAATAATCAGTTCCTC |
| | | CCATATCCATATTATGCCAAACCGGCCGCAGTCAGATCACCA |
| | | GCGCAGATATTGCAGTGGCAGGTCCTTTCCAACACAGTGCCG |
| | | GCTAAGAGCTGCCAAGCTCAACCGACGACAATGGCCCGCCA |
| | | CCCTCATCCACACCTGAGCTTCATGGCGATCCCCCCCAAGAA |
| | | AAATCAAGACAAGACGGAAATTCCGACCATCAATACAATAGCT |
| | | TCAGGCGAACCTACAAGTACTCCTACAACTGAAGCGGTCGAG |
| | | TCGACTGTTGCGACCCTGGAAGATTCTCCCGAGGTGATTGAG |
| | | AGCCCTCCAGAGATCAACACCGTGCAGGTGACGAGTACTGC |
| | | CGTA |
| 31 | (CymB; trunc) | GCGGAAATAACCCGCATCCCGCTCTAGAAGGGGAAAAGTTTG |
| | | CGGAAAGCTCTGAAGGAGCATGGACTGCTGGAGGATTTCCT |
| | | GCAGAAGCAGCAATATGGAATATCCTCAAAGTACTCTGGTTTT |
| | | GGGGAGGTGGCGTCAGTTCCCTTAACCAACTACCTTGATTCA |
| | | CAGTACTTCGGGAAGATTTACCTCGGCACTCCTCCTCAAGAG |
| | | TTTACGGTTCTATTTGATACTGGTTCCTCGGACTTCTGGGTTC |
| | | CGTCCATCTACTGCAAATCTAATGCTTGCAAGAACCACCAGA |
| | | GGTTCGACCCGCGAAAGAGCTCGACGTTTCAAAATCTTGGCA |
| | | AACCTCTCTCTATCCACTACGGCACCGGCTCCATGCAGGGCA |
| | | TACTGGGTTATGATACAGTGACCGTCAGCAATATTGTAGACAT |
| | | TCAACAAACAGTCGGGCTATCTACCCAAGAACCCGGTGATGT |
| | | TTTCACCTATGCAGAGTTCGACGGTATTCTCGGCATGGCCTA |
| | | CCCATCGTTAGCTAGCGAGTATAGCATACCGGTGTTTGATAA |
| | | CATGATGAATCGCCATCTCGTCGCGCAGGACCTGTTCTCCGT |
| | | GTACATGGATAGAAATGGACAGGAGTCCATGCTGACTTTGGG |
| | | TGCTATTGATCCCTCTTATTATACTGGCTCACTTCACTGGGTG |
| | | CCGGTCACGGTGCAGCAGTACTGGCAGTTCACTGTTGATTCT |
| | | GTTACAATCAGCGGCGTCGTCGTAGCATGTGAAGGCGGCTG |
| | | CCAGGCGATTCTTGACACCGGAACAAGTAAATTGGTTGGGCC |
| | | GAGCAGCGACATCCTCAATATCCAGCAGGCCATTGGAGCAAC |
| | | GCAGAACCAGTATGGCGAATTTGATATTGACTGTGACAACTT |
| | | GTCCTACATGCCAACAGTGGTGTTTGAGATCAACGGCAAGAT |
| | | GTATCCACTTACTCCATCGGCATATACATCTCAAGATCAAGGC |
| | | TTCTGCACGTCAGGATTCCAGTCAGAAAATCATAGTCAAAAGT |
| | | GGATCTTGGGCGACGTGTTCATCCGTGAGTACTACAGCGTCT |
| | | TCGACCGCGCCAACAACCTGGTAGGGCTCGCCAAGGCCATA |
| 32 | Myoglobin (MYG; | GGGTTAAGTGATGGAGAATGGCAGCTGGTGCTCAATGCATG |
| | trunc) | GGGCAAGGTGGAGGCAGATGTGGCGGGTCACGGCCAAGAA |
| | | GTTCTCATCAGGCTCTTTACCGGTCATCCAGAGACGCTGGAG |
| | | AAGTTTGACAAGTTCAAGCATCTGAAAACAGAAGCAGAGATG |
| | | AAGGCCAGCGAGGATCTGAAGAAACACGGTAACACTGTCCTT |
| | | ACAGCTCTGGGCGGCATTTTGAAGAAGAAGGGACACCATGAA |
| | | GCCGAGGTCAAACATTTGGCAGAATCTCACGCCAACAAGCAC |
| | | AAGATACCGGTAAAATATCTAGAGTTCATCAGCGACGCCATC |
| | | ATTCACGTCCTGCACGCCAAGCACCCTTCAGACTTCGGCGCT |
| | | GACGCTCAAGCGGCCATGTCCAAAGCGCTGGAGCTGTTCCG |
| | | CAATGATATGGCCGCGCAGTACAAGGTTCTTGGGTTTCATGG |
| | | A |
| 33 | Alpha hemoglobin | GTGCTCTCGGCCGCCGACAAAGGGAACGTCAAAGCGGCGTG |
| | (HBA; trunc) | GGGCAAGGTCGGCGGCCATGCTGCTGAGTATGGTGCTGAAG |
| | | CTCTTGAAAGGATGTTCTTGAGTTTTCCTACAACGAAAACCTA |
| | | CTTCCCACATTTTGATTTGAGTCATGGATCAGCACAGGTGAA |
| | | GGGTCACGGGGCCAAGGTGGCGGCCGCGCTCACAAAAGCA |
| | | GTGGAGCACCTCGACGACCTTCCTGGAGCATTATCGGAGCT |
| | | GAGCGACCTGCACGCGCACAAGCTAAGAGTTGATCCAGTCA |
| | | ACTTCAAGCTGCTGTCCCACAGCCTGCTGGTAACGCTGGCCA |
| | | GCCATTTGCCGTCTGATTTTACCCCCGCCGTCCATGCAAGCC |
| | | TAGACAAGTTCCTCGCTAATGTTTCTACTGTTCTTACTTCCAA |
| | | GTACCGC |
| 34 | Alpha actin | TGCGACGAGGATGAAACCACCGCTCTTGTTTGTGACAACGGC |
| | (ACTA; trunc) | TCGGGCCTCGTGAAGGCCGGGTTTGCTGGTGATGATGCACC |
| | | CCGCGCGGTATTCCCCTCTATTGTCGGGCGGCCGCGCCACC |
| | | AGGGCGTGATGGTTGGCATGGGTCAAAAGGATAGCTACGTG |
| | | GGTGATGAGGCGCAGAGTAAACGTGGCATACTGACCCTAAA |
| | | GTACCCAATTGAACATGGCATCATCACCAACTGGGACGACAT |
| | | GGAAAAAATTTGGCACCACACGTTCTACAATGAGCTTCGAGT |
| | | GGCCCCGGAGGAGCATCCAACTCTCCTAACTGAAGCACCGC |
| | | TCAACCCCAAGGCAAATAGGGAGAAGATGACACAAATAATGT |
| | | TTGAGACATTCAACGTGCCTGCGATGTATGTGGCTATACAAG |
| | | CTGTTTTGTCACTGTACGCCAGTGGAAGAACAACTGGAATTG |
| | | TACTGGACTCAGGTGACGGCGTCACCCACAATGTTCCTATAT |
| | | ATGAAGGCTATGCACTGCCGCACGCCATCATGCGCCTTGATC |
| | | TTGCTGGACGCGACCTCACCGACTACCTAATGAAGATTTTGA |
| | | CAGAAAGAGGATACAGCTTTGTCACTACGGCCGAGAGGGAG |
| | | ATCGTGAGGGATATCAAAGAAAAGCTCTGCTACGTTGCGCTC |
| | | GACTTCGAGAACGAGATGGCCACGGCGGCGAGCTCCTCTAG |
| | | TCTGGAGAAATCTTATGAACTGCCTGATGGGCAGGTGATCAC |
| | | TATAGGAAATGAAAGATTTCGGTGCCCCGAGACCTTGTTTCA |
| | | ACCTAGTTTCATAGGGATGGAGTCAGCGGGCATTCATGAAAC |
| | | GACTTACAATTCCATCATGAAATGTGATATCGATATTAGGAAA |
| | | GACCTGTATGCCAACAATGTCATGTCGGGGGGTACAACGATG |
| | | TACCCTGGGATTGCTGATAGGATGCAGAAGGAGATAACTGCT |
| | | TTGGCACCATCTACCATGAAGATCAAGATTATCGCCCCGCCA |
| | | GAACGTAAATATTCTGTCTGGATTGGAGGGTCGATTCTTGCC |
| | | AGCCTGTCCACATTCCAGCAGATGTGGATCACAAAGCAGGAG |
| | | TACGACGAGGCGGGCCCGTCCATCGTTCATCGGAAGTGCTT |
| | | C |

The DNA sequences encoding the maize gamma zein signal (mZ27; SEQ ID NO: 23; encoded by SEQ ID NO: 24) and KDEL (SEQ ID NO: 25; encoded by SEQ ID NO: 26) peptides were added respectively to the 5′ (amino-, N-terminal sequence of the protein) and the 3′ (carboxy-, C-terminal sequence of the protein) ends of the maize codon optimized DNA sequences. The native bovine protein molecules do not possess either the gamma zein signal peptide, nor the KDEL (SEQ ID NO: 25) sequence and do not accumulate in plants to similar levels when lacking the gamma zein signal and KDEL (SEQ ID NO: 25) sequence. The modified proteins (mZ27:TargetProtein:KDEL (SEQ ID NO: 25), where “TargetProtein” represents the protein sequence (without the native signal peptide) of either the alpha-S1-casein precursor, alphaS2-casein, beta-casein, kappa-casein, chymosin, myoglobin, hemoglobin, or actin alpha subunit as described in Table 2) were maize codon optimized using the GenSmart tool to provide nucleotide sequences encoding these proteins for plant expression. The optimized food-related bovine sequences, where underlined sequences at their N-terminal ends denote signal peptide, are listed in Table 4:

TABLE 4
Amino acid sequences of optimized food-related bovine
proteins expressed in maize

| SEQ | | | |
|---|---|---|---|
| ID | Bovine modified | Accession | |
| NO | protein name | Number | Amino acid sequence |
| 35 | Alpha-S1-casein | NP_851372.1 | MRVLLVALALLALAASATSRPKHPIKHQGLPQ |
| | (CasA1; opt) | | EVLNENLLRFFVAPFPEVFGKEKVNELSKDIG |
| | | | SESTEDQAMEDIKQMEAESISSSEEIVPNSVE |
| | | | QKHIQKEDVPSERYLGYLEQLLRLKKYKVPQL |
| | | | EIVPNSAEERLHSMKEGIHAQQKEPMIGVNQE |
| | | | LAYFYPELFRQFYQLDAYPSGAWYYVPLGTQ |
| | | | YTDAPSFSDIPNPIGSENSEKTTMPLWKDEL |
| 36 | Alpha-S2-casein | NP_776953.1 | MRVLLVALALLALAASATSKNTMEHVSSSEESI |
| | (CasA2; opt) | | ISQETYKQEKNMAINPSKENLCSTFCKEVVRN |
| | | | ANEEEYSIGSSSEESAEVATEEVKITVDDKHY |
| | | | QKALNEINQFYQKFPQYLQYLYQGPIVLNPWD |
| | | | QVKRNAVPITPTLNREQLSTSEENSKKTVDME |
| | | | STEVFTKKTKLTEEEKNRLNFLKKISQRYQKFA |
| | | | LPQYLKTVYQHQKAMKPWIQPKTKVIPYVRYL |
| | | | KDEL |
| 37 | Beta-casein | AAA30480.1 | MRVLLVALALLALAASATSRELEELNVPGEIVE |
| | (CasB; opt) | | SLSSSEESITRINKKIEKFQSEEQQQTEDELQD |
| | | | KIHPFAQTQSLVYPFPGPIPNSLPQNIPPLTQT |
| | | | PVVVPPFLQPEVMGVSKVKEAMAPKHKEMPF |
| | | | PKYPVEPFTESQSLTLTDVENLHLPLPLLQSW |
| | | | MHQPHQPLPPTVMFPPQSVLSLSQSKVLPVP |
| | | | QKAVPYPQRDMPIQAFLLYQEPVLGPVRGPF |
| | | | PIIVKDEL |
| 38 | Kappa-casein | NP_776719.1 | MRVLLVALALLALAASATSQEQNQEQPIRCEK |
| | (CasK; opt) | | DERFFSDKIAKYIPIQYVLSRYPSYGLNYYQQK |
| | | | PVALINNQFLPYPYYAKPAAVRSPAQILQWQV |
| | | | LSNTVPAKSCQAQPTTMARHPHPHLSFMAIPP |
| | | | KKNQDKTEIPTINTIASGEPTSTPTTEAVESTV |
| | | | ATLEDSPEVIESPPEINTVQVTSTAVKDEL |
| 39 | Chymosin B | P00794 | MRVLLVALALLALAASATSAEITRIPLYKGKSLR |
| | (CymB; opt) | | KALKEHGLLEDFLQKQQYGISSKYSGFGEVAS |
| | | | VPLTNYLDSQYFGKIYLGTPPQEFTVLFDTGS |
| | | | SDFWVPSIYCKSNACKNHQRFDPRKSSTFQN |
| | | | LGKPLSIHYGTGSMQGILGYDTVTVSNIVDIQQ |
| | | | TVGLSTQEPGDVFTYAEFDGILGMAYPSLASE |
| | | | YSIPVFDNMMNRHLVAQDLFSVYMDRNGQES |
| | | | MLTLGAIDPSYYTGSLHWVPVTVQQYWQFTV |
| | | | DSVTISGVVVACEGGCQAILDTGTSKLVGPSS |
| | | | GILNIQQAIGATQNQYGEFDIDCDNLSYMPTVV |
| | | | FEINGKMYPLTPSAYTSQDQGFCTSGFQSEN |
| | | | HSQKWILGDVFIREYYSVFDRANNLVGLAKAIK |
| | | | DEL |
| 40 | Myoglobin (MYG; | P02192 | MRVLLVALALLALAASATSGLSDGEWQLVLNA |
| | opt) | | WGKVEADVAGHGQEVLIRLFTGHPETLEKFD |
| | | | KFKHLKTEAEMKASEDLKKHGNTVLTALGGIL |
| | | | KKKGHHEAEVKHLAESHANKHKIPVKYLEFIS |
| | | | DAIIHVLHAKHPSDFGADAQAAMSKALELFRN |
| | | | DMAAQYKVLGFHGKDEL |
| 41 | Alpha hemoglobin | P01966 | MRVLLVALALLALAASATSVLSAADKGNVKAA |
| | (HBA; opt) | | WGKVGGHAAEYGAEALERMFLSFPTTKTYFP |
| | | | HFDLSHGSAQVKGHGAKVAAALTKAVEHLDD |
| | | | LPGALSELSDLHAHKLRVDPVNFKLLSHSLLVT |
| | | | LASHLPSDFTPAVHASLDKFLANVSTVLTSKY |
| | | | RKDEL |
| 42 | Alpha actin | P68138 | MRVLLVALALLALAASATSCDEDETTALVCDN |
| | (ACTA; opt) | | GSGLVKAGFAGDDAPRAVFPSIVGRPRHQGV |
| | | | MVGMGQKDSYVGDEAQSKRGILTLKYPIEHGI |
| | | | ITNWDDMEKIWHHTFYNELRVAPEEHPTLLTE |
| | | | APLNPKANREKMTQIMFETFNVPAMYVAIQAV |
| | | | LSLYASGRTTGIVLDSGDGVTHNVPIYEGYAL |
| | | | PHAIMRLDLAGRDLTDYLMKILTERGYSFVTTA |
| | | | EREIVRDIKEKLCYVALDFENEMATAASSSSLE |
| | | | KSYELPDGQVITIGNERFRCPETLFQPSFIGME |
| | | | SAGIHETTYNSIMKCDIDIRKDLYANNVMSGGT |
| | | | TMYPGIADRMQKEITALAPSTMKIKIIAPPERKY |
| | | | SVWIGGSILASLSTFQQMWITKQEYDEAGPSI |
| | | | VHRKCFKDEL |

Expression constructs were developed that contain maize codon optimized DNA sequences encoding the modified alpha-S1-casein, alphaS2-casein, beta-casein, kappa-casein, chymosin, myoglobin, hemoglobin, and actin alpha proteins, containing mZ27 and KDEL peptides added to the amino- and carboxy-termini of the proteins, respectively. The coding sequences for these proteins were synthesized as BamHI-AvrII DNA fragments and cloned between selected seed specific gene promoters such as the rice glutelin-1 (Gtl1), the rice glutelin B-4 (GluB4), or the maize gamma zein 27 kDa (gZein) promoter, and transcriptional termination sequences from either nopaline synthase (NOS) or cauliflower mosaic virus (T35S). The resulting expression cassettes (SEQ ID NO: 43-52) are described in Tables 5 and 6.

TABLE 5
Description of the individual genetic elements in developed gene
expression cassettes cloned into the maize transformation vectors

| SEQ | Vector/ | Genetic elements | | | |
|---|---|---|---|---|---|
| ID | expression | | Signal | Coding | Termi- |
| NO | cassette | Promoter | peptide | sequence | nator |
| 43 | pAG5009 | Rice | Maize gamma | CymB | T35S |
| | | Glutelin-1 | zein 27 | | |
| 44 | pAG5011 | Rice | Maize gamma | CasA1 | T35S |
| | | Glutelin-1 | zein 27 | | |
| 45 | pAG5012 | Rice glutelin | Maize gamma | CasB | NOS |
| | | B-4 | zein 27 | | |
| 46 | pAG5013 | Maize | Maize gamma | CasK | NOS |
| | | gamma zein | zein 27 | | |
| | | 27 | | | |
| 47 | pAG5014 | Maize | Maize gamma | CasA2 | NOS |
| | | gamma zein | zein 27 | | |
| | | 27 | | | |
| 48 | pAG5016, | Rice | Maize gamma | MYG | T35S |
| | pAG5020^{a} | Glutelin-1 | zein 27 | | |
| 49 | pAG5017 | Rice | Maize gamma | HBA | T35S |
| | | Glutelin-1 | zein 27 | | |
| 50 | pAG5018 | Rice | Maize gamma | ACTA | T35S |
| | | Glutelin-1 | zein 27 | | |
| 51 | pAG5019 | Rice glutelin | Maize gamma | MYG | NOS |
| | | B-4 | zein 27 | | |
| 52 | pAG5020^{b} | Rice glutelin | Maize gamma | MYG | T35S |
| | | B-4 | zein 27 | | |
| 64 | pAG5024^{c} | Rice | Maize gamma | MYGm | T35S |
| | | Glutelin-1 | zein 27 | | |
| 65 | pAG5025^{d} | Rice glutelin | Maize gamma | MYGm | T35S |
| | | B-4 | zein 27 | | |
| 67 | pAG5027^{e} | Rice | Maize GluTR | HvGluTR | T35S |
| | | Glutelin-1 | | | |
| 68 | pAG5028^{f} | Maize | Maize FC1A | ZmFC1A | NOS |
| | | gamma zein | | | |
| | | 27 | | | |
| 70 | pAG5030^{h} | Barley Ltp2 | Maize gamma | MYG | NOS |
| | | | zein 27 | | |
| 71 | pAG5031 | Barley Ltp2 | Maize gamma | EU591743 | NOS |
| | | | zein 27 | | |
| 72 | pAG5032^{g} | Maize AI9 | Maize gamma | MYG | NOS |
| | | | zein 27 | | |
| 73 | pAG5033 | Maize AI9 | Maize gamma | EU591743 | NOS |
| | | | zein 27 | | |
| 74 | pAG5034 | Soybean | Maize gamma | MYG | NOS |
| | | CG1 | zein 27 | | |
| 76 | pAG5036 | Maize AI9 | none | MYG | AI9T |
| 77 | pAG5037 | Maize AI9 | Maize AI9 | MYG | NOS |

^{a}2^{nd} expression cassette in pAG5020
^{b}1^{st} expression cassette in pAG5020
^{c}1^{st} expression cassette in pAG5026
^{d}2^{nd} expression cassette in pAG5026
^{e}1^{st} expression cassette in pAG5029
^{f}2^{nd} expression cassette in pAG5029
^{g}1^{st} expression cassette in pAG5035
^{h}2^{nd} expression cassette in pAG5035

TABLE 6
Nucleotide coordinates of individual genetic elements in
developed gene expression cassettes described in Table 5.

| SEQ | Vector/ | Nucleotide coordinates of genetic elements within cassette | | | |
|---|---|---|---|---|---|
| ID | Expression | | Signal | Coding | |
| NO | cassette | Promoter | peptide | sequence | Terminator |
| 43 | pAG5009 | 1-2071 | 2081-2137 | 2138-3247 | 3254-3433 |
| 44 | pAG5011 | 1-2071 | 2081-2137 | 2138-2749 | 2756-2935 |
| 45 | pAG5012 | 1-1474 | 1484-1540 | 1541-2182 | 2189-2464 |
| 46 | pAG5013 | 1-1363 | 1373-1429 | 1430-1951 | 1958-2233 |
| 47 | pAG5014 | 1-1363 | 1373-1429 | 1430-2065 | 2072-2374 |
| 48 | pAG5016, | 1-2071 | 2081-2137 | 2138-2611 | 2618-2797 |
| | pAG5020* | | | | |
| 49 | pAG5017 | 1-2071 | 2081-2137 | 2138-2575 | 2582-2761 |
| 50 | pAG5018 | 1-2071 | 2081-2137 | 2138-3280 | 3287-3466 |
| 51 | pAG5019 | 1-1474 | 1484-1540 | 1541-2014 | 2021-2296 |
| 52 | pAG5020** | 1-1474 | 1484-1540 | 1541-2014 | 2021-2200 |
| 64 | pAG5024 | 1-2071 | 2081-2137 | 2138-2611 | 2618-2797 |
| 65 | pAG5025 | 1-1474 | 1484-1540 | 1541-2014 | 2021-2296 |
| 66 | pAG5026 | 2207-4277 | 4287-4343 | 4344-4817 | 4824-5003 |
| | (cassette 1) | | | | |
| | pAG5026 | 1-1474 | 1484-1540 | 1541-2014 | 2021-2200 |
| | (cassette 2) | | | | |
| 67 | pAG5027 | 1-2071 | 2081-2233 | 2234-3598 | 3605-3784 |
| 68 | pAG5028 | 1-1363 | native | 1373-2827 | 2834-3109 |
| 69 | pAG5029 | 3116-5186 | 5196-5348 | 5349-6713 | 6720-6899 |
| | (cassette 1) | | | | |
| | pAG5029 | 1-1363 | 1373-1433 | 1434-2827 | 2834-3109 |
| | (cassette 2) | | | | |
| 70 | pAG5030 | 1-1501 | 1511-1567 | 1568-2041 | 2048-2323 |
| 71 | pAG5031 | 1-1501 | 1514-1570 | 1571-2170 | 2177-2452 |
| 72 | pAG5032 | 1-2553 | 2563-2619 | 2620-3093 | 3100-3375 |
| 73 | pAG5033 | 1-2553 | 2566-2622 | 2623-3222 | 3229-3504 |
| 74 | pAG5034 | 1-968 | 978-1034 | 1035-1508 | 1515-1790 |
| 75 | pAG5035 | 2330-4882 | 4892-4948 | 4949-5422 | 5429-5704 |
| | (cassette 1) | | | | |
| | pAG5035 | 1-1501 | 1511-1567 | 1568-2041 | 2048-2323 |
| | (cassette 2) | | | | |
| 76 | pAG5036 | 1-2553 | none | 2563-3027 | 3034-4033 |
| 77 | pAG5037 | 1-2553 | 2563-2619 | 2620-3081 | 3088-4087 |

*2^{nd} expression cassette in pAG5020
**1^{st} expression cassette in pAG5020

The individual expression cassettes were cloned into pAG4500- or pAG4003-based plasmids to create vectors for expressing the modified protein sequences in maize grain. The vector pAG4500 (SEQ ID NO: 53) is a basic cloning vector for maize gene expression and contains a single expression cassette for the phosphomannose isomerase (PMI) selectable marker. Another basic vector that was used for cloning is pAG4003. This vector is identical to pAG4500 (SEQ ID NO: 53) except for a short nucleotide sequence representing the multiple cloning site. The nucleotide sequences differences between vectors pAG4003 and pAG4500 (SEQ ID NO: 53) are provided below in the pairwise alignment (the aligned sequences are referenced by the nucleotides 10100-10205 in SEQ ID NO: 53).

| | |
|---|---|
| pAG4003 | CTCAGCTTAA-------------------------------------------------- |
| pAG4500 | CTCAGCCTAAGCGGCCGCATTGGACTTAATTAAGTGAGGCCGGCCAAGCGTCGATTTAAA 10159 |
| | ****** *** |
| pAG4003 | ------------------------------------TTAAGTCTAA (SEQ ID NO: 55) |
| pAG4500 | TGTACCACATGGCGCGCCAACTATCATGCGATCGCTTCATGTCTAA (SEQ ID NO: 54) 10205 |
| | * * ****** |

In total, twenty-three vectors containing individual cassettes for expressing bovine proteins were constructed. The vectors pAG5012, pAG5019, pAG5025 and pAG5026 were cloned into the pAG4003 and the vectors pAG5009, pAG5011, pAG5013, pAG5014, pAG5016, pAG5017, pAG5018, pAG5024, pAG5027-pAG5037 into the pAG4500. The list of these vectors and description of corresponding expression cassettes with specific nucleotide positions of individual genetic elements within these cassettes is provided in Table 6.

Three double stack vectors pAG5020, pAG5026, and pAG5035 were developed for heightening expression levels of bovine myoglobin in maize grain. It is known that the presence of multiple transgene copies has a positive effect on increasing transgene expression. The pAG5020, pAG5026 and pAG5035 have two myoglobin expression cassettes that are cloned into the same T-DNA fragment facilitating simultaneous insertion of two myoglobin gene copies, expressed from two different promoters, into genomes of transformed maize plants. Specifically, pAG5020 contains the entire intact MYG expression cassette from the vector pAG5016 (SEQ ID NO: 48) and a modified MYG cassette from the vector pAG5019 (SEQ ID NO: 51) in which the originally cloned NOS terminator was replaced by the T35S terminator sequence and the newly cloned cassette was designated as SEQ ID NO: 52 (thus, pAG5020 contains SEQ ID NOS: 48 and 52, and has the sequence of SEQ ID NO: 56 for both cassettes). In order to construct double stack vector pAG5020, the vector pAG5016 was digested with PmeI-KpnI restriction enzymes and used as acceptor vector for simultaneous cloning of two DNA fragments: 1) PmeI-AvrII fragment isolated from pAG5019 and containing terminator-less MYG expression cassette; 2) AvrII-KpnI fragment containing T35S terminator sequence. The cloned pAG5020 contains pAG4003 vector backbone.

The pAG5026 contains the entire intact MYGm expression cassette from the vector pAG5024 (SEQ ID NO: 64) and a modified MYGm cassette from the vector pAG5025 (SEQ ID NO: 65) in which the originally cloned NOS terminator was replaced by the T35S terminator sequence and the newly cloned cassette was designated as SEQ ID NO: 66 (thus, pAG5026 contains SEQ ID NOS: 64 and 65, and has the sequence of SEQ ID NO: 66 for both cassettes). The pAG5035 contains the entire intact MYG expression cassette from the vector pAG5032 (SEQ ID NO: 72) and the entire MYG cassette from the vector pAG5030 (SEQ ID NO: 70), and the newly clone cassette was designated as SEQ ID NO: 75. The double stack vectors pAG5026 and pAG5035 were cloned in a similar way that is described for pAG5020.

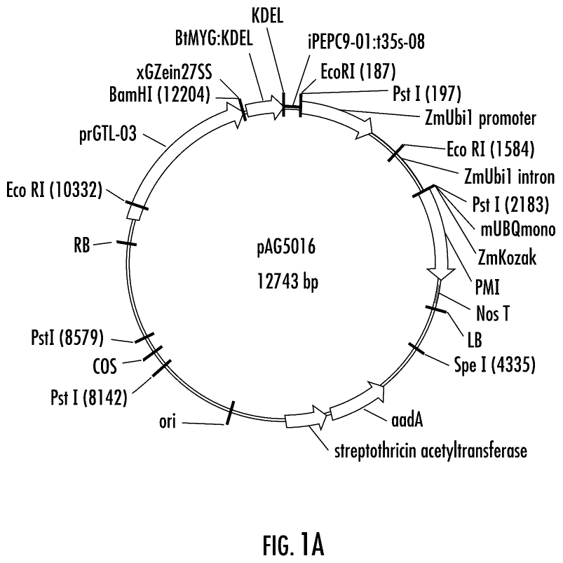

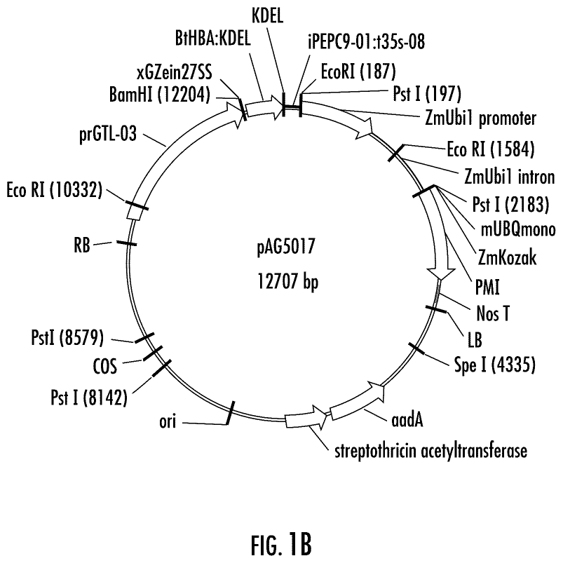

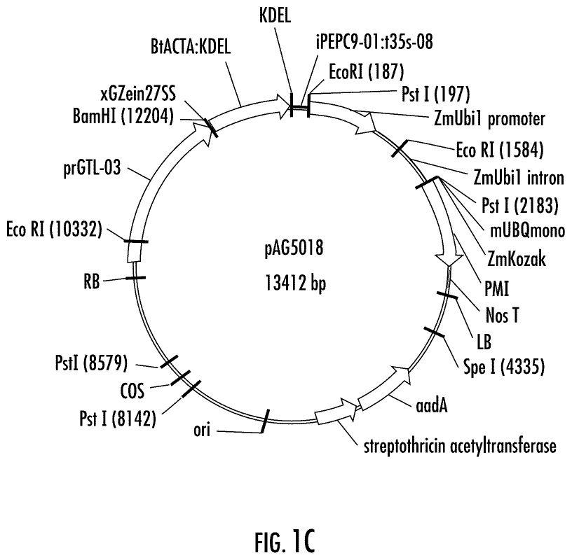

FIGS. 1A-1C illustrate tandem expression cassettes for myoglobin, hemoglobin, and actin in respective vectors pAG5016 (FIG. 1A; SEQ ID NO: 48), pAG5017 (FIG. 1B; SEQ ID NO: 49) and pAG5018 (FIG. 1C; SEQ ID NO: 50). Each one of the expression cassettes includes GTL-03, promoter; Z27ss, signal peptide from maize gamma zein 27; specific gene coding sequence; KDEL (SEQ ID NO: 25), tetrapeptide C-terminal signal sequence for retention in the endoplasmic reticulum; iPEPC9-01:t35s-08 terminator sequence.

Promoters: maize globulin 1 promoter, maize oleosin 16, maize PEP carboxylase, maize ubiquitin 1 (with intron), maize gamma zein 27, rice glutelin 1 (SEQ ID NO: 87), and rice glutelin B4 (SEQ ID NO: 88), prHvLtp2 promoter (SEQ ID NO: 83; DNA, prZmAI9 (SEQ ID NO: 84; DNA) and prGmCG1 (Seq ID NO: 85; DNA).

Signal peptides: maize gamma zein 27, rice glutelin, maize A19 signal sequence (SEQ ID NO: 62; AA) and (SEQ ID NO: 82; DNA), soybean alpha subunit beta-conglycinin signal sequence (SEQ ID NO: 63; AA).

Coding nucleic acid sequences and amino acid sequences of myoglobin: (SEQ ID NOS: 32 and 78 (DNA) and SEQ ID NOS: 1-3, 20, 40 and 58 (AA)).

Coding nucleic acid sequences and amino acid sequences of hemoglobin: (SEQ ID NO: 33 (DNA) and SEQ ID NOS: 4-6, 21, and 41 (AA)).

Coding nucleic acid sequences and amino acid sequences of actin: (SEQ ID NO: 34 (DNA) and SEQ ID NOS: 7-9, 22, and 42 (AA)).

Coding nucleic acid sequences and amino acid sequences of casein: (SEQ ID NO: 27-30 (DNA), and SEQ ID NOS: 10-13, 15-18, and 35-38 (AA)).

Coding nucleic acid sequences and amino acid sequences of chymosin: (SEQ ID NOS: 31 (DNA) and SEQ ID NOS: 14, 19, and 39 (AA)).

Coding nucleic acid sequences and amino acid sequences of glutamyl-tRNA reductase: (SEQ ID NO: 80 (DNA) and SEQ ID NO: 59 (AA)).

Coding nucleic acid sequences and amino acid sequences of ferrochelatase: (SEQ ID NO: 81 (DNA), and SEQ ID NO: 61 (AA)).

C-terminal extensions: HvVSD (from the Hordeum vulgare vacuolar sorting determinant (Cervelli et al., 2004)); SEKDEL (SEQ ID NO: 57); KDEL (SEQ ID NO: 25; Endoplasmic reticulum retention signal; (Arakawa, Chong, & Langridge, 1998; Haq, Mason, Clements, & Arntzen, 1995; Korban, 2002; Munro & Pelham, 1987)).

Terminators/polyadenylation signals: NOS (from the Agrobacterium tumefaciens nopaline synthase gene), CaMV 35s (from the cauliflower mosaic virus 35s transcript; the sequence includes an intron from the maize PEP carboxylase gene)), maize globulin 1; ZmAI9 terminator (SEQ ID NO: 86).

### Example 2. Plant Transformation and Analysis

Maize Transformation: Maize embryos were transformed with casein, chymosin, myoglobin, hemoglobin, and actin constructs pAG5009, pAG5011, pAG5012, pAG5013, pAG5014, pAG5016, pAG5017, pAG5018, pAG5019, pAG5020, pAG5026, pAG5029, pAG5030, pAG5032, pAG5034, pAG5035, pAG5036 and/or pAG5037 according to Negrotto D. et al. 2000 Plant Cell Rep 19: 798; Ishida Y et al. 1996 Nat Biotech 14: 74, which is incorporated herein by reference as if fully set forth.

Briefly, embryogenic callus from wild-type A×B maize was inoculated with LBA4404 Agrobacterium cells harboring the appropriate transformation plasmid. Agrobacterium-mediated transformation of immature maize embryos was performed as described on Negrotto D. et al. The expression cassettes were cloned into an intermediate vector capable of recombining with the pSB1 vector in triparental mating in Agrobacterium tumefaciens strain LBA4404 using procedures reported previously (Ishida Y et al. 1996 Nat Biotech 14: 745; Hiei Y et al. 1994 Plant J 6: 271; Hiei Y and Komari T 2006 Plant Cell Tissue Organ Cult. 85: 27; Komari T et al. 1996 Plant J 10: 165). Maize (Zea mays cultivars Hill, A188 or B73) stock plants were grown in a greenhouse under 16 hours of daylight at 28° C. Immature zygotic embryos were isolated from the kernels and inoculated with the Agrobacterium solution containing the genes of interest. After inoculation immature embryos were grown in a tissue culture process for 10-12 weeks. Well-developed seedlings with leaves and roots were sampled for PCR analysis to identify transgenic plants containing the genes of interest. PCR positive and rooted plants were rinsed with water to wash off the agar medium, and transplanted to soil and grown in the greenhouse to generate seeds.

Plant DNA Isolation for 96 well plates: Briefly, a COSTAR grinding block was filled with three-fourths leaf samples, one 5 mm steel bead was added to each well with a sample and the storage mat block was applied using a storage mat applicator to seal the block. Samples were stored at −80° C. for at least 30 min before grinding or until processing time. For processing, samples were ground for 45 sec using the Klecko Pulverizer & Secure grinder at maximum speed. Sealing was removed and discarded. Three hundred microliters of 10XTE+Sarkosyl buffer (5 mL 1M Tris, 1 mL 0.5M EDTA, 0.5 g sarkosyl, 46 mL ddH₂O) was added to each sample using a multichannel pipette and a sterile solution basin. The plate was incubated on a shaker for 10 min at 300 rpm, and spun for 3 min at 4000 rpm. Supernatant was removed and discarded, and the pellets were resuspended in 1×TE buffer. One hundred fifty microliter sample aliquots were added to the 96 well PCT plate. The PCR plate was sealed with aluminum foil. For best results, DNA isolation and PCR were performed on the same day.

Transgenic Diagnostic PCR Reaction Setup: The “complete” PCR reaction mix was as follows: 15 μl 2× GoTaq MM (GoTaq Green Master Mix (PROMEGA #M712), 3 μl of combined forward and reverse primers specific to the gene of interest (each mixed at 10 μM), 2 μl DNA template preparation, and water to adjust volume to 30 μl. Twenty-eight microliters of the “complete” PCR reaction mix per well were aliquoted into a PCR plate (FISHER, #14230236). Two microliters plant DNA sample were aliquoted into each well of the PCR plate. Positive control and no template negative control were used in each PCR reaction. Control Agrobacterium DNA was diluted 1:100 in TE buffer to yield clear bands. The PCR plate was sealed with a sealing mat (COSTAR #6555) and roller. PCR was performed at BIORAD PT8C-100 thermocycler. The thermocycler programs were as follows: 1) 95° C.—3 min; 30 cycles of 95° C.—30 sec, 55° C.—30 sec, 72° C.—45 sec; 72° C.—5 min; 10° C. (hold), and 2) 90° C. for 30 min and 10° C. (hold). Twelve microliters of each PCR reaction was loaded onto Ready Agarose 96 Plus gel −3% (BIORAD #161-3062) and ran at approximately 100V for 20 minutes before viewing with a BIORAD gel doc system equipped with Quantity One software. Quick-Load 50 bp DNA Ladder (NEB N0473S) was used to identify the size of the PCR fragments. 10×TBE Buffer (Promega V4251) was used.

### Example 3. Expression of Casein, Chymosin, Myoglobin, Hemoglobin, and Actin in Transgenic Plants

Independently transgenic maize plants that had been transformed with vectors as described above were grown to maturity, and cross-pollinated with wild-type (untransformed) maize plants. Approximately 20 seed were harvested from each of these plants. Seeds were milled through a 0.5 mm screen to produce a fine powder. Protein was then extracted, mixed with 2× Laemmli Sample Buffer, heated for five minutes at 95 C prior to loading onto polyacrylamide gels (30 μg/lane) and run at 200 V for one hour in 10% SDS-PAGE Criterion (Biorad) gel using Tris/Glycine/SDS running buffer at pH 8.3. After electrophoresis the gels were rinsed three times for five minutes with water in order to remove SDS, stained for one hour in 50 mL (per gel) of Bio-Safe Coomassie Brilliant Blue G-250 stain and destained in water for one hour. Expression of the recombinant protein was compared against protein standards: alpha-casein (Sigma Aldrich, Catalog #C6780), beta-casein (Sigma Aldrich, Catalog #C6905), kappa-casein (Sigma Aldrich, Catalog #C0406), chymosin (Sigma Aldrich, Catalog #R4877), myoglobin (equine myoglobin from skeletal muscle was used as a standard, which has a similar molecular weight to the bovine myoglobin, Sigma Aldrich, Catalog #M0630), hemoglobin (Sigma Aldrich, Catalog #H2500), actin (Sigma Aldrich, Catalog #A3653). Grain protein extracts were also assayed using an appropriate ELISA kit as described below.

ELISA assay from seed. Protein extracts were prepared by incubating 15 mg milled seed flour for 1 hour at room temperature in 1.5 ml of extraction buffer (25 mM sodium borate, pH 10, 0.01% Tween 20). Extracts were then serially diluted 10-fold to 10,000-fold in ELISA buffer as described in the corresponding kit. The rest of the ELISA assay was performed according to the manufacturer's instructions. Casein alpha S1 expression was measured by ELISA using an ELISA kit (Aviva Systems Biology, Catalog #OKCD05288) per the manufacturer's instructions. Casein alpha S2 expression was measured by ELISA using an ELISA kit (Aviva Systems Biology, Catalog #OKEH08310) per the manufacturer's instructions. Beta casein expression was measured by ELISA using an ELISA kit (Aviva Systems Biology, Catalog #OKCD02467) per the manufacturer's instructions. Kappa casein expression was measured by ELISA using an ELISA kit (Aviva Systems Biology, Catalog #OKEH07974) per the manufacturer's instructions. Chymosin expression was measured using an ELISA kit (Signalway Antibody, Catalog #EK18611) per the manufacturer's instructions. Myoglobin was measured using an ELISA kit (Cusabio, CSB-EL013529BO) per the manufacturer's instructions. Hemoglobin was measured using an ELISA Kit (Amsbio, Catalog #AMS.E11H1362) per the manufacturer's instructions. Actin was measured using an ELISA Kit (Amsbio, Catalog #AMS-E11A2152) per the manufacturer's instructions.

A range of expression levels may be observed among transgenic plants that had been generated with the expression vectors. Common expression levels in maize corn grain for casein alpha S1, casein alpha S2, beta casein, kappa casein, chymosin A, chymosin B, myoglobin, hemoglobin, and actin may be less than 1 mg of recombinant protein per gram of grain (hemizygous). Some events for each expressed protein may exceed 1 mg per gram of grain, some may exceed 3 mg per gram of grain, and expression levels up to 12 mg per gram of grain may be observed. Based on the observed expression levels, homozygous grain could provide recombinant protein production at levels of up to 25 mg of recombinant protein per gram of grain. The recombinant protein activity may be detectable in grain from independently-transformed transgenic maize plants. Plant IDs are arbitrary numerical tags assigned to individual plants for the purpose of tracking the corresponding plants and their progeny.

### Heme Availability in Maize Endosperm to Support Charging of Myoglobin and Hemoglobin Proteins

Carotenoids are a class of pigments that are synthesized in many plant tissues, not the least of which is maize endosperm. Carotenoid synthesis involves cytochrome P450-type (CYP) monoxygenases (Cuttriss et al. 2011). CYP monooxygenases are heme containing enzymes (Saxena et al. 2013). From this, it is apparent that heme is available in maize endosperm. Further illustrating this point is the work of Proulx (Proulx 2007), who expressed maize hemoglobin in maize endosperm tissue and showed that the recombinant hemoglobin incorporated heme. These observations demonstrate that recombinant globin proteins such as myoglobin and hemoglobin will incorporate heme as they accumulate in maize endosperm.

In experiments measuring myoglobin and hemoglobin expression in maize grain, we also showed that these recombinant proteins were charged with heme using a method similar to that described previously (Bonfils et al, 1995). Incorporation of the iron pyrrole into the recombinantly produced myoglobin or hemoglobin expressed in transgenic maize was accomplished using the pseudo-peroxidase activity of the iron associated with the heme pyrrole, which was combined with luminol based chemiluminescence. Briefly, 200 mg of ground corn grain, positive for either myoglobin or hemoglobin based on ELISA measurements (see above), was extracted for 30 min with shaking in 1 mL 100 mM Tris, pH 6.8, and centrifuged. The supernatant was concentrated by ultrafiltration through a 10,000 MWCO filtration device (Millipore Ultrafree MC), then 50 μL±5 μL of the filter retentate was mixed with 200 μL of luminol buffer (1 mL 0.1 mol/L luminol, 1 mL 0.02 mol/L 4-iodophenol, and 20 μL 30% hydrogen peroxide in 50 mL phosphate buffered saline, pH 7.4). Chemiluminescence was measured on a spectrophotometer and heme iron concentrations were estimated by comparing with myoglobin or hemoglobin standards using log-log curve fitting.

### Example 4. Properties of the Recombinant Proteins

Gastric stability of recombinant casein, chymosin, myoglobin, hemoglobin, and actin. Gastric lability of a recombinant protein is one factor that is considered when governments contemplate permitting propagation of crops in the wild that produce a recombinant protein. To determine how casein, chymosin, myoglobin, hemoglobin, and actin might persist in the digestive tract, specifically as it passes through the stomach, protein standards were purified and subjected to a standardized assay for sensitivity to simulated gastric fluid (Thomas et al., 2004).

### Example 8. PCR Assays for Identifying and Determining Zygosity of Transgenic Events

Maize events carry transgenes that result in seed-specific expression of the recombinant protein. Some events carry multiple T-DNAs at a single genetic locus, whereas other events carry multiple T-DNAs, each at different genetic loci. Molecular identification and tracking of these transgenes can be done using standard (endpoint, gel-based) PCR or real-time PCR. In addition to determining whether a plant is carrying a transgene, these assays can also determine whether a plant is null, hemizygous (carrying one copy of the/each insertion) or homozygous (carrying two copies of the/each insertion).

### Example 9. Expressing Codon Optimized Casein, Chymosin, Myoglobin, Hemoglobin, or Actin Sequences

Enhanced recombinant protein expression in transgenic grain can provide an added value to the final product by lowering the necessary acreage required to produce any specified amount of protein, thereby reducing the operations required for identity preservation of the grain, and by reducing the amount of necessary storage and transportation for a given mass of protein, all of which lower the cost of the protein. Comparative analysis of protein expression in transgenic seed can reveal that the median level of casein, chymosin, myoglobin, hemoglobin, and actin expression in transgenic lines, as it is assessed by ELISA, is higher when expressed using coding sequences that are codon optimize for maize from otherwise identical constructs (containing unoptimized sequences). The improved sequences can be cloned into plant expression vectors designed for seed specific expression and transformed into maize. The seeds from generated transgenic events can be analyzed for recombinant protein expression by ELISA. The resulting expression data is expected to demonstrate a clear improvement expression from the optimized sequences, with multiple events demonstrating specific activities in hemizygous seed greater than events generated using unoptimized sequences.

### Example 10. Plant Expressed Chymosin Validation

Chymosin activity was detected using a milk-clotting assay, similar to those described elsewhere (Luo, Jiang, Yang, Li, & Jiang, 2016; Wei et al., 2016). The substrate was non-fat milk purchased from a local grocery store. Commercial chymosin (“Liquid Chymosin Rennet”) was purchased from the New England Cheese Making Supply Company (Northampton MA). A 1:100 dilution of the commercial chymosin was prepared in deionized water.

Original transformants made with pAG5009 were grown to maturity. Pollen collected from individual transformants was used to pollinate silks from wild-maize type plants (inbred variety 1-11054). Mature ears were harvested and dried at ambient temperature. Approximately 20 kernels were collected from each ear and milled to a coarse powder in an IKA Tube Mill. Approximately 100 mg of powder from each sample was transferred to a microcentrifuge tube and extracted with 1 ml deionized water at 37° C. for 1 hour, with gentle agitation. Samples were then pelleted in a microcentrifuge for 1 minute, and 100 μl aliquots of the supernatants were used in the milk clotting tests.

Microcentrifuge tubes containing 1 ml of non-fat milk were pre-warmed to 30° C. for 30 minutes. Subsequently, the following were added to individual tubes:
1. Nothing (negative control)
2. 100 μl deionized water
3. 100 μl 0.1M sodium acetate buffer (pH5.0)
4. 1 μl 1:100 dilution of commercial chymosin (positive control)
5. 100 μl grain extract from an unrelated transgenic plant
6. 100 μl grain extract from ear #F1Z_5009_5, expressing chymosin
7. 100 μl grain extract from ear #F1Z_5009_6, expressing chymosin

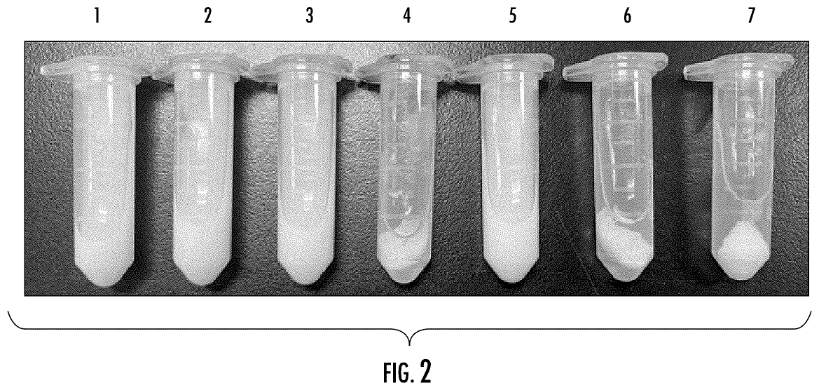

μMicrocentrifuge tubes were then incubated at 30° C. with gentle agitation for 19 hours. Subsequently, the samples were centrifuged for 1 min to precipitate the coagulated milk products. FIG. 2. shows extracts from chymosin-expressing grain can coagulate milk. Tubes are numbered as described above.

Whereas milk (tube 1) and milk that had been supplemented with either water (tube 2) or sodium acetate buffer (tube 3) was stable for 19 hours at 30° C., a small amount of commercial chymosin was sufficient to cause clotting (tube 4). Extracts of grain from two independent transformants that carry transgenes for expressing chymosin similarly clotted the milk (tubes 6 and 7), while an extract from grain that did not express chymosin was unable to clot milk (tube 5).

### Example 11. Plants Expressing Casein

Casein protein accumulation was detected using Western blot analysis. In the assay, sodium carbonate buffer extracts from the transgenic samples yielded (on average) about 50 mg protein per g of milled grain. 100 μg of total soluble protein was loaded per lane onto the gel. Approximately 5-10 ng of recombinant protein per sample was obtained which corresponds to 2.5-5.0 μg casein per gram of grain.

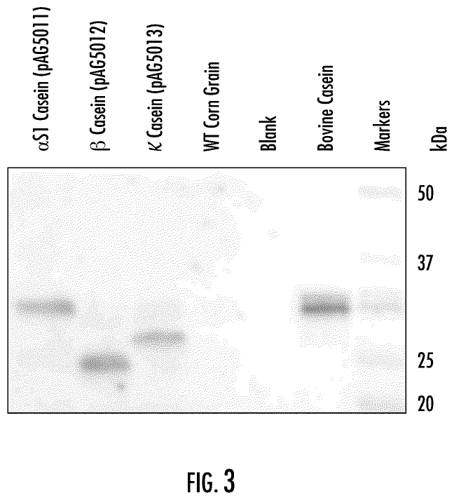

FIG. 3 is photograph showing Western blot data for casein candidates. In the figure, the lanes correspond to the following samples (from left to right): αS1-casein (pAG5011), β-casein (pAD5012), κ-casein (pAG5013), WT corn grain, blank, bovine casein and markers. In the Western image, the casein control corresponds to 10 ng of protein (based on the A280 in the nanodrop device). Among the pAG5011 plants (alpha-S1 casein), 13 candidates had a range of concentrations from 1-20 ng casein per 100 μg total soluble protein. Among the pAG5012 plants (beta casein), 8 candidates ranged from 1-10 ng casein per 100 μg soluble protein. Among the pAG5013 plants (kappa caseins), two candidates had 0.5-1.0 ng casein per 100 μg soluble protein, while one had 5-10 ng casein per 100 μg soluble protein.

### Example 12. Sequences and Vectors for Expressing Myoglobin in Maize

Enhancing expression levels of recombinant bovine myoglobin in the maize grain is an important method for providing low-cost, high-quality, myoglobin protein for food, feed, and pharmaceutical applications. Myoglobin is a heme-containing protein, and like many heme-containing proteins, myoglobin's folding, stability, and function are dependent upon heme availability and incorporation into the protein. Myoglobin can be expressed in different plant tissues and cell types. Within maize grain, myoglobin can be expressed specifically in the aleurone layer of cells, embryo, or in the endosperm. It's possible to specifically express myoglobin in any one of these tissues on its own, or to express it in any combination, including simultaneously in all three areas.

It is well known that the protein accumulation within the maize grain varies between different tissues. In this regard, the aleurone consists of only a single layer of cells and makes up to 2.2% of seed dry matter (Wolf et al., 1972), but aleurone can accumulate approximately 19.2% of total protein (Hilton, 1953) or up to 30% of the endosperm proteins. Furthermore, it was demonstrated that aleurone cells contain enhanced levels of carotenoids (Ndolo and Beta, 2013) and some enzymes of the carotenoid biosynthetic pathway require heme for their function (Zhai et al., 2016), similar to myoglobin. Thus, heme is available in aleurone cells and there is at least some functioning level of the tetrapyrrole biosynthetic pathway inside aleurone cells. Because of the presence of heme in aleurone cells, there may be adequate heme production capacity to achieve high levels of myoglobin accumulation, in which case there would be little need for metabolic engineering of the heme-biosynthetic pathway in aleurone cells. Likewise, even if heme is present in maize grain, if its concentration is limiting in the formation of functional and stable myoglobin, then increasing heme biosynthesis would be helpful to achieving higher levels of myoglobin expression. Whatever the heme availability may be in the seeds, the presence of heme should improve the proper folding and function of bovine myoglobin expressed to this group of cells as well as potentially enhance overall recombinant protein production in maize grain. Additionally, expressing a recombinant protein in the aleurone layer of cells could be useful for mitigating undesirable effects on grain characteristics that can occur, when certain proteins are expressed in the endosperm (for example, xylanase and amylase can adversely effect seed morphology, which can impact grain yield and quality).

In maize, at least one aleurone-specific promoter of the Betl9like gene was identified and characterized (Royo et al., 2014). A shorter version of this promoter was described earlier in US Patent Application Publication 20030097689 A1, published May 22, 2003. The gene encodes an orthologous protein to the barley End-2 lipid transfer protein. The Betl9like gene is expressed in developing maize seeds in the outer surface of the maize kernels. It was determined by in situ hybridization that its protein accumulation is specific for the aleurone layer of cells. The gene is located on chromosome 8 and is designated as GRMZM2G091054 (a19), while the encoded protein is available at the GenBank as an accession number AQK99894.

A maize aleurone specific 2553 bp promoter prZmAI9 was cloned as a sequence positioned on the maize chromosome 8 between nucleotides 178066845-178064293. This promoter region includes the previously characterized 2229 bp sequence of the Betl9like gene (Royo et al., 2014). The Betl9like (end2) protein expressed from this promoter is 100% identical in amino acid sequence to an UniProt entry B4FFB8 that is designated as AL-9 protein and encoded by the al-9 gene. The amino acids 1-24 in AL-9 protein are annotated as a signal sequence.

The barley Ltp2 (HvLtp2) promoter 801 bp or 807 bp sequences have been functionally characterized by Kalla et al., 1994 and Olsen et al., 1996. It was demonstrated by particle bombardment experiments that the Ltp2 promoter drives GUS gene expression exclusively in aleurone layer of the rice grain. This promoter provides ˜5% of the GUS activity of the strong ubiquitous rice actin gene promoter. The barley Ltp2 gene and promoter sequence is available in the GenBank as an accession number X69793. In order to extend the promoter sequence into 5′ end direction, the sequence X69793 was used to BLASTN publicly available barley genome sequence database. The BLASTN analysis identified two sequence contigs that originated from barley cultivars Golden Promise and Morex and containing the Ltp2 gene with its promoter. The Clustal Omega multiple sequence alignment software program was used to align Ltp2 sequences from the identified contigs with the original X69793 sequence. The multiple sequence alignment revealed that the barley Ltp2 sequence from Golden Promise contig is 100% identical to the X69793 sequence, while the similar sequence specific to the Morex contig is divergent at its 3′ promoter end comparing to X69793. The Morex sequence also has a 6 bp mutation in the Ltp2 coding region leading to deletion of two amino acids from the predicted LTP2 protein sequence (see multiple sequence alignments below). The 1858 bp upstream of the coding Ltp2 region sequence from Golden Promise contig was subjected to promoter motif search using available online tool PlantPAN3.0. Based on this analysis, a 1.5 kb promoter sequence (prHvLtp2) from the Golden Promise contig was selected for development of vectors for expressing bovine myoglobin in the aleurone layer of the maize grain.

In order to increase availability of heme in endosperm, where bovine myoglobin is expressed from under endosperm-specific promoters of rice Glutelin 1 (SEQ ID NO: 87) and rice GluB4 (SEQ ID NO: 88) cassettes cloned into vectors pAG5016 (SEQ ID NO: 48), pAG5019 (SEQ ID NO: 51), pAG5020 (SEQ ID NO: 56), pAG5024 (SEQ ID NO: 64), pAG5025 (SEQ ID NO: 65) and pAG5026 (SEQ ID NO: 66), a truncated by 30 amino acids at the N-terminal end variant (SEQ ID NO: 59) of the barley glutamyl-tRNA reductase HvGluTRm (HEMA1, accession number X86101) as well as the maize ferrochelatase FC1A were expressed in the maize seed under the rice glutelin-1 gene or the maize gamma zein 27 kDa gene promoters. Glutamyl-tRNA reductase and ferrochelatase have been shown to be the rate limiting enzymes in plant heme production, and their recombinant expression has been shown to increase heme availability in plants. It was demonstrated that the removal of the first 30 amino acids from mature barley GluTR not only maintains its enzymatic activity, but also abolishes metabolic feedback inhibition of the glutamyl-tRNA reductase by heme (Vothknecht et al., 1998). For expressing in maize grain, the barley HvGluTRm was fused to the maize GluTR signal sequence (SEQ ID NO: 60). The maize ferrochelatase FC1A was demonstrated to be functional in heme biosynthesis (Woodson et al., 2011). The FC1A protein sequence is deposited to NCBI as an accession number ACG39230. The gene encoding this protein is located on the maize chromosome 5 (LOC100284108) and its sequence is available at NCBI as an accession number NM_001157005. To facilitate cloning ZmFC1A, a silent mutation (G429>A) in the codon for Arg143 was introduced in order to eliminate the AvrII restriction site.

The nucleotide sequence of the soybean promoter prGmCG1 that drives expression of the alpha′ subunit of the soybean beta-conglycinin was identified within the GenBank accession M13759.

The coding regions of BtMYGm, HvGluTRm, ZmAI9ss:BtMYG and ZmFC1A were maize codon optimized and synthesized by the GenScript as BamHI-AvrII DNA fragments; promoters prZmAI9, prHvLtp2 and prGmCG1 were synthesized as KpnI-BamHI fragments; the maize terminator ZmAI9T was prepared as AvrII-EcoRI DNA fragment. Cloning of these genetic elements was performed essentially as described in Example 1 herein.

The vectors pAG5031 and pAG5033 expressing xylanase EU591743 from the aleurone specific promoters prZmAI9 and prHvLtp2 were constructed as the control vectors to validate functionality of promoters. The procedure for determining activity of the EU591743 xylanase in transgenic maize grain is readily available.

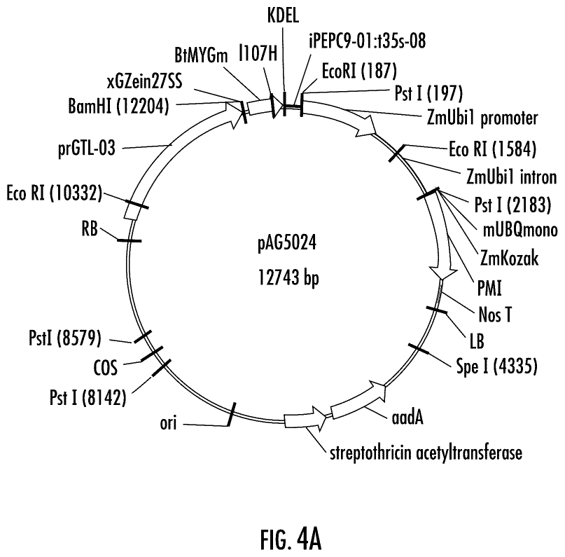

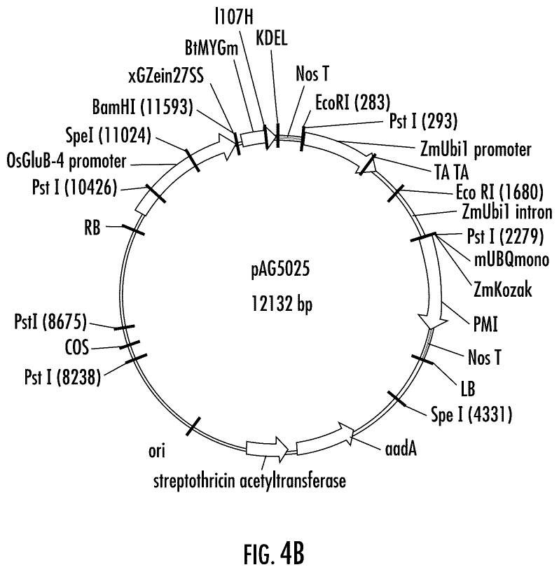

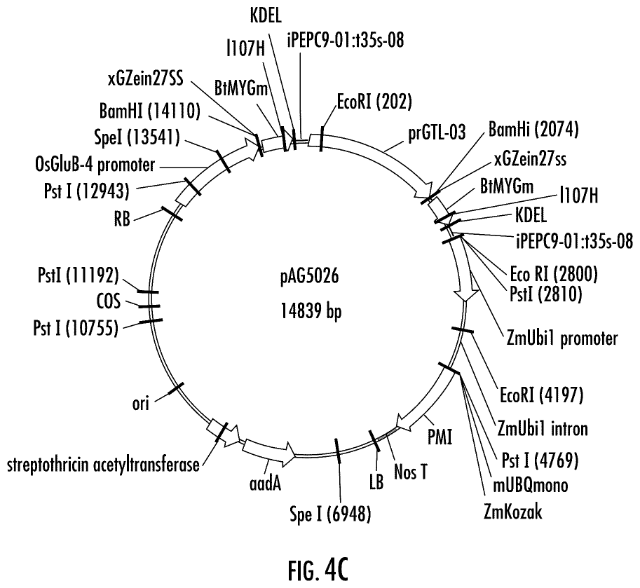

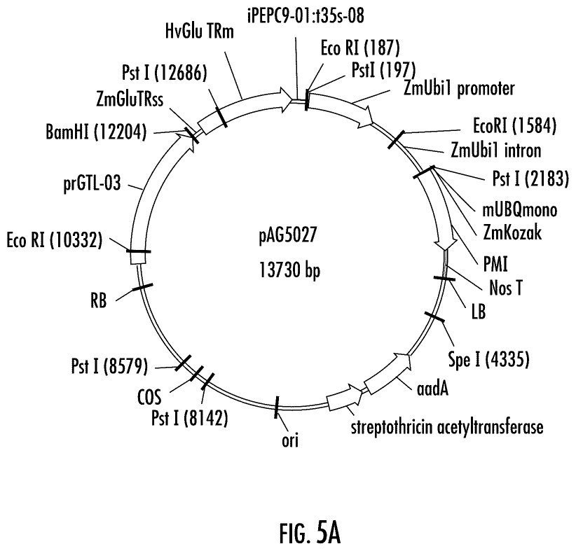

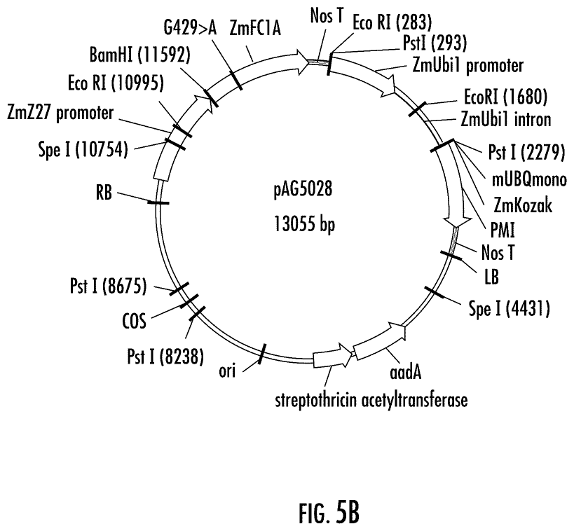

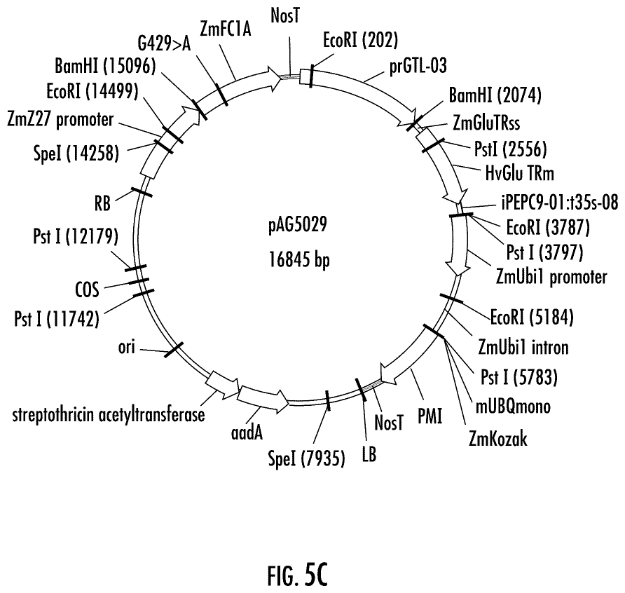

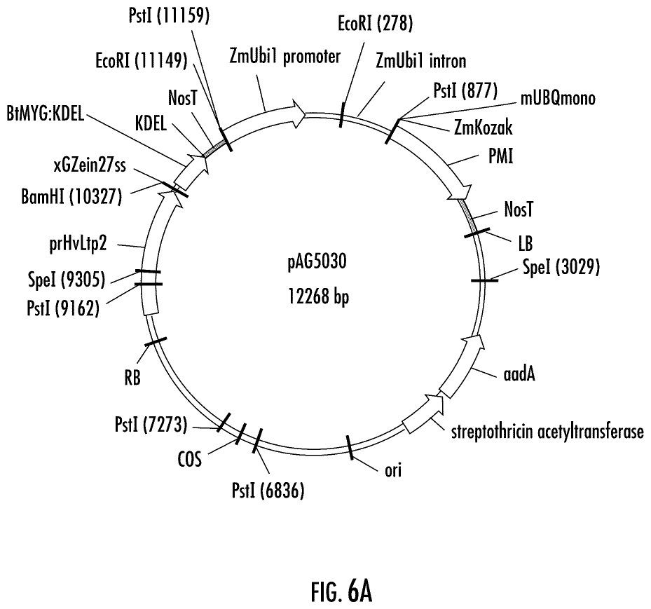

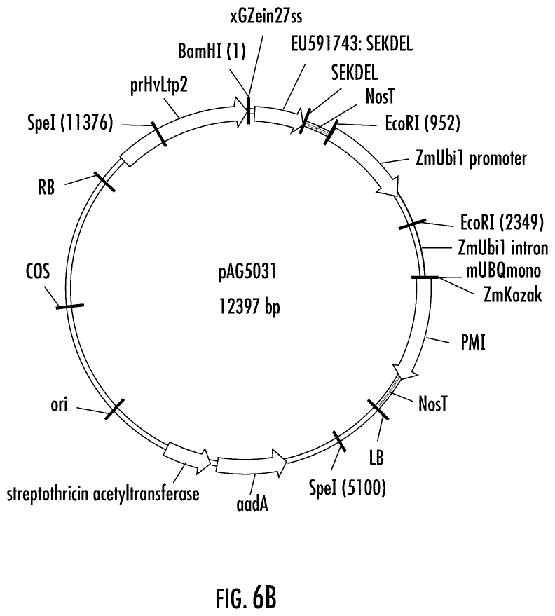

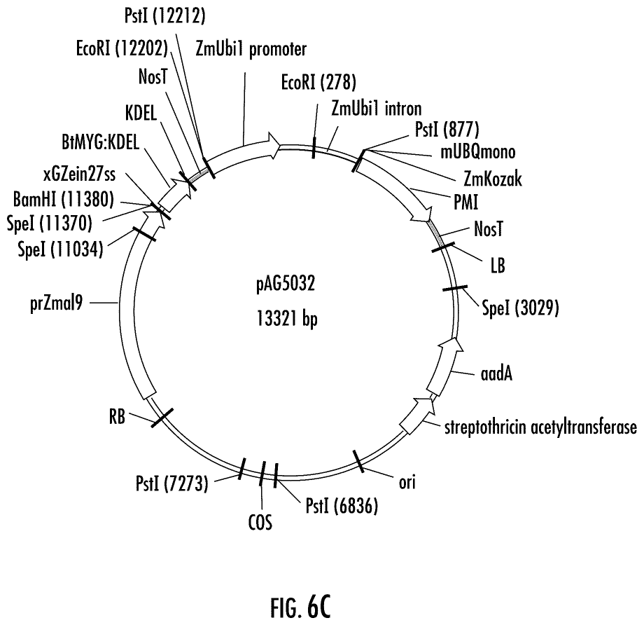

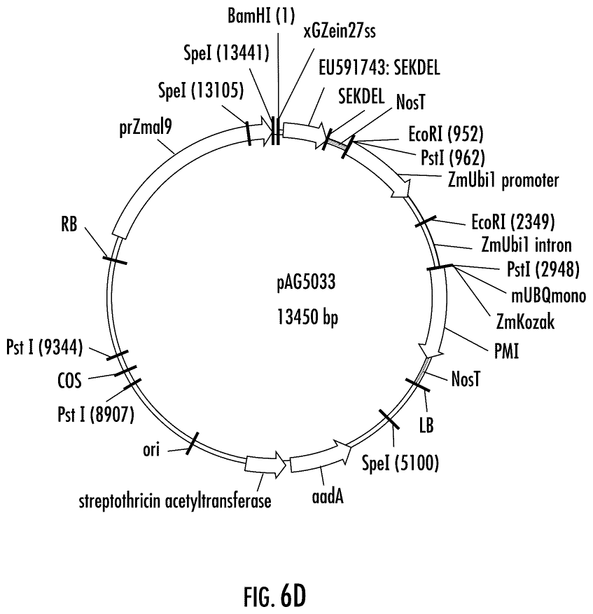

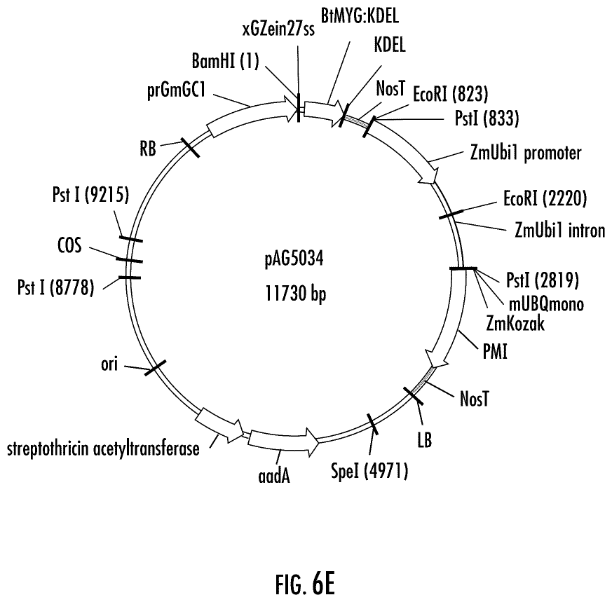

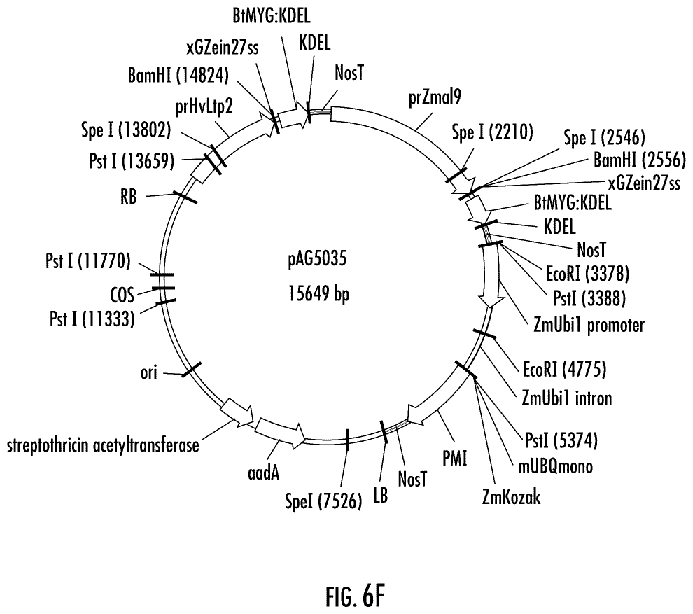

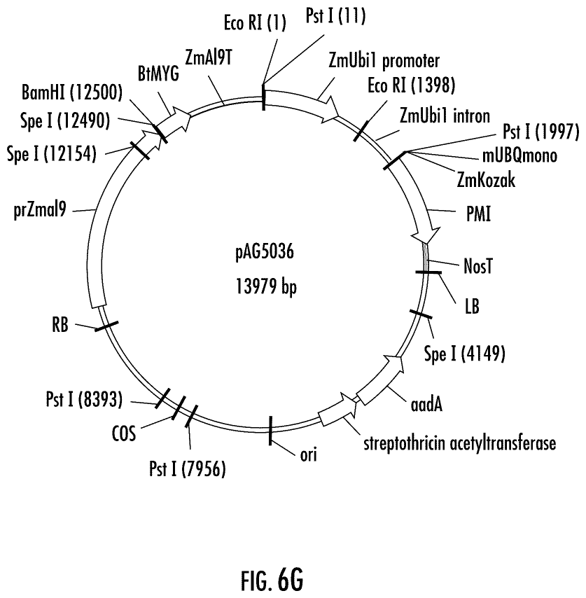

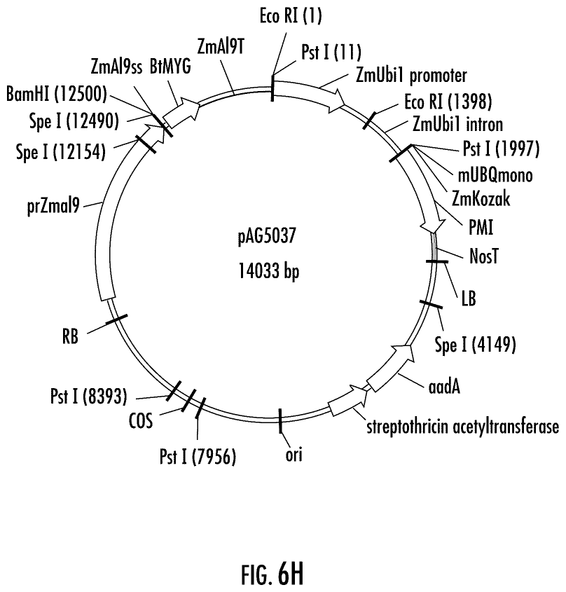

In order to express bovine myoglobin in the maize seed expression cassettes were constructed and vectors were developed as shown in FIGS. 4A-4C, 5A-5C, 6A-6H. Expression cassettes for mutated myoglobin are contained in vectors pAG5024 (FIG. 4A), pAG5025 (FIG. 4B), pAG5026 (FIG. 4C) and expression cassettes for myoglobin is also contained in vector pAG5034 (FIG. 6E). Likewise, the expression cassettes for increasing heme biosynthesis in endosperm are contained in vectors pAG5027 (FIG. 5A), pAG5028 (FIG. 5B), pAG5029 (FIG. 5C). Similarly, the expression cassettes for the bovine myoglobin and EU591743 xylanase expression in aleurone layer of the maize seed in vectors pAG5030 (FIG. 6A), pAG 5031 (FIG. 6B), pAG5032 (FIG. 6C), pAG5033 (FIG. 6D), pAG5035 (FIG. 6F), pAG5036 (FIG. 6G), and pAG5037 (FIG. 6H).

Specifically, FIG. 4A illustrates vector pAG5024 containing the mutated bovine myoglobin expression cassette that includes GTL-03 promoter; Z27ss, signal peptide from maize gamma zein 27; Bos taurus mutated myoglobin (BtMYGm) gene coding sequence; KDEL (SEQ ID NO: 25), and iPEPC9-01:t35s-08 terminator sequence. FIG. 4B illustrates vector pAG5025 containing the mutated bovine myoglobin expression cassette that includes OsGluB-4 promoter; Z27ss, signal peptide from maize gamma zein 27; BtMYGm gene coding sequence; KDEL (SEQ ID NO: 25), and NOS terminator. FIG. 4C illustrates vector pAG5026 that includes two expression cassettes: one cassette including OsGluB-4 promoter; Z27ss, signal peptide from maize gamma zein 27; BtMYGm gene coding sequence; KDEL (SEQ ID NO: 25); and another cassette including GTL-03 promoter; Z27ss, signal peptide from maize gamma zein 27; BtMYGm gene coding sequence; KDEL (SEQ ID NO: 25) and iPEPC9-01:t35s-08 terminator sequences for both cassettes.

FIG. 5A illustrates vector pAG5027 containing the expression cassette that includes GTL-03 promoter; ZmGluTRss signal peptide from the maize glutamyl tRNA reductase; barley glutamyl-tRNA reductase HvGluTRm gene coding sequence; and iPEPC9-01:t35s-08 terminator sequence. FIG. 5B illustrates vector pAG5028 containing the expression cassette that includes Zea mays gamma zein promoter Z27, Maize ferrochelatase ZmFC1A coding sequence and NOS terminator. FIG. 5C illustrates vector pAG5029 that includes two expression cassettes: on cassette including Zea mays gamma zein promoter Z27, Maize ferrochelatase ZmFC1A coding sequence and NOS terminator; and another cassette including GTL-03 promoter; ZmGluTRss signal peptide; HvGluTRm gene coding sequence; and iPEPC9-01:t35s-08 terminator sequence.

FIG. 6A illustrates vector pAG5030 that includes the expression cassette containing the barley prHvLtp2 promoter, signal peptide from maize gamma zein 27, bovine myoglobin BtMYG coding sequence; KDEL (SEQ ID NO: 25) and NOS terminator.

FIG. 6B illustrates vector pAG5031 that includes the expression cassette containing the barley prHvLtp2 promoter, signal peptide from maize gamma zein 27, EU591743 xylanase coding sequence; SEKDEL (SEQ ID NO: 57) and NOS terminator.

FIG. 6C illustrates vector pAG5032 that includes the expression cassette containing the maize aleurone specific prZmAI9 promoter, signal peptide from maize gamma zein 27, bovine myoglobin BtMYG coding sequence; KDEL (SEQ ID NO: 25) and NOS terminator. FIG. 6D illustrates vector pAG5033 the expression cassette containing the that includes maize aleurone specific prZmAI9 promoter, signal peptide from maize gamma zein 27, EU591743 xylanase coding sequence; SEKDEL (SEQ ID NO: 57) and NOS terminator. FIG. 6E illustrates vector pAG5034 that includes the expression cassette containing the soybean promoter prGmCG promoter, signal peptide from maize gamma zein 27, bovine myoglobin BtMYG coding sequence; KDEL (SEQ ID NO: 25) and NOS terminator. FIG. 6F illustrates vector pAG5035 that includes two cassettes: one cassette including barley prHvLtp2 promoter, signal peptide from maize gamma zein 27, bovine myoglobin BtMYG coding sequence; KDEL (SEQ ID NO: 25) and NOS terminator; and another cassette including maize aleurone specific prZmAI9 promoter, signal peptide from maize gamma zein 27, bovine myoglobin BtMYG coding sequence; KDEL (SEQ ID NO: 25) and NOS terminator. FIG. 6G illustrates vector pAG5036 that includes the expression cassette containing the maize aleurone specific prZmAI9 promoter, bovine myoglobin BtMYG coding sequence; and ZmAI9 terminator. FIG. 6H illustrates vector pAG5037 that includes the expression cassette containing the maize aleurone specific prZmAI9 promoter, ZmAI9ss signal peptide, bovine myoglobin BtMYG coding sequence; and ZmAI9 terminator.

### Example 13. Description of the Mutant Form of Myoglobin

Heme dissociates from some hemoglobins more readily than from others, with a hemoglobin from Synechocystis PCC 6803 having the strongest heme binding identified to-date, due to the presence of an additional covalent linkage between the protein and the heme prosthetic group (Uppal et al., 2015). In this linkage heme is covalently bound to an additional, non-canonical histidine in the heme binding pocket of the protein. Synechococcus PCC 7002 has a similar truncated hemoglobin with the additional non-canonical histidine residue (Becana, Yruela, Sarath, Catal An, & Hargrove, 2020). Using a model for the tertiary structure of the Synechocystis hemoglobin, it has been possible to introduce a histidine into an analogous position in sperm whale myoglobin, which improved the strength of the bond between the recombinant myoglobin and the heme prosthetic group without significantly changing the oxygen or carbon monoxide binding kinetics of the protein (Uppal et al., 2015). There is sufficient homology between sperm whale and bovine myoglobins to identify the position that would enable a similar modification to the bovine protein as shown in homology alignment below.

The homology observation provided the basis for introducing an analogous change into the bovine myoglobin sequence, as described below (the BtMYG gene), resulting in a mutant gene BtMYGm encoding the modified protein BtMYGm. It is possible that such a strong linkage between the protein and the prosthetic group makes it more difficult for the cell to degrade or recycle the protein (Uppal et al., 2015), which may allow it to accumulate more readily. Introducing a similar mutation into a plant-derived hemoglobin, such as leghemoglobin, would have a similar effect of binding heme more tightly. However, the lack of strong homology between the primary sequences of mammalian myoglobins and plant hemoglobins suggest that this exercise may not be straightforward. Nonetheless, the crystal structure of a small number of plant hemoglobins, including at least one from maize, have been solved (Becana et al., 2020). Conservation of the tertiary structures of many hemoglobins suggests that it may be possible to use this information to identify analogous positions within the plant hemoglobins for introducing non-canonical histidines. Alignment of Bovine Myoglobin (BtMYG; SEQ ID No: 1) vs Sperm Whale Myoglobin (PcMYG; GenPept ID NP_001277651.1), where dots in the sperm whale protein sequence indicate identity with the bovine sequence, and uppercase text indicates amino acids that differ. The underlined amino acid in the BtMYG sequence indicates the isoleucine residue that was changed to histidine in the mutant form of myoglobin

| | | | |
|---|---|---|---|
| BtMYG | 1 | MGLSDGEWQLVLNAWGKVEADVAGHGQEVLIRLFTGHPETLEKFDKFKHLKTEAEMKASE | 60 |
| PCMYG | 1 | .V..E.......HV.A..........DI......KS.........R.............. | 60 |
| BtMYG | 61 | DLKKHGNTVLTALGGILKKKGHHEAEVKHLAESHANKHKIPVKYLEFISDAIIHVLHAKH | 120 |
| PcMYG | 61 | ......V.......A...........L.P..Q...T.....I.......E.......SR. | 120 |
| BtMYG | 121 | PSDFGADAQAAMSKALELFRNDMAAQYKVLGFHG (SEQ ID NO: 1) | 154 |
| PcMYG | 121 | .G.......G..N.......K.I..K..E..YQ. | 154 |

### Example 14. Expression of Myoglobin in Zea mays

Following transformation, individual transformed plants (“T0s”) were grown to maturity. Pollen was collected from each TO and used to pollinate silks on wild-type (untransformed) maize plants, and the resulting seed were tested for the accumulation of myoglobin via western blot. Briefly, seed from individual “WT×T0” crosses, as described above, were ground to a fine powder. 500 μl of sodium carbonate/bicarbonate buffer, pH 10.8, was added to 100 mg of powder from each sample, and proteins were extracted at room temperature for 1 hour with gentle agitation. Approximately 3 ul of each extract was loaded onto an 8-16% Criterion™ TGX™ Precast Midi Protein Gel (BioRad, Hercules CA) and separated via polyacrylamide gel electrophoresis. Proteins were subsequently blotted to an Immun-Blot® PVDF Membrane (BioRad) and probed, using a rabbit anti-bovine myglobin antibody (AbCam, Cambridge UK) as a primary antibody, and an HRP-conjugated donkey anti-rabbit secondary antibody (Agrisera, Vsnnss, Sweden). Detection was carried out using SuperSignal™ West Dura Extended Duration Substrate (Thermo Scientific, Bedford MA).

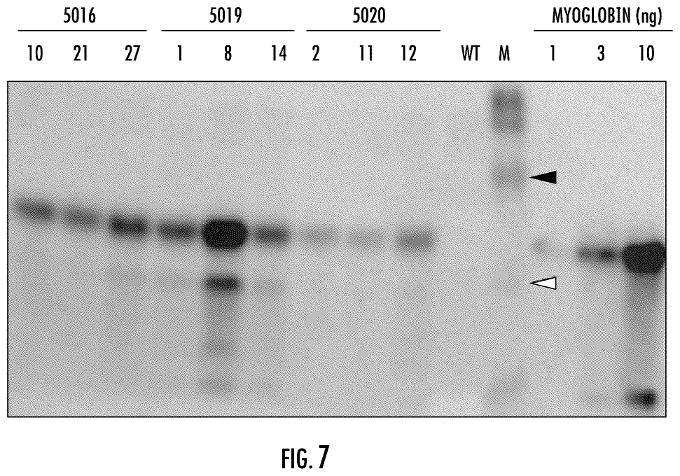

FIG. 7 is a photograph of the Western blot that shows detection of myoglobin in extracts of seed derived from individual plants that had been transformed with T-DNAs carrying myoglobin expression constructs. In the figure, Lanes 1-3, TO candidates #10, 21 and 27 carrying T-DNAs from pAG5016. Lanes 4-6, candidates #1, 8 and 14 carrying T-DNAs from pAG5019. Lanes 7-9, candidates #2, 11 and 12 carrying T-DNAs from pAG5020. WT, extracts derived from self-pollinated wild-type (untransformed) plants. M, molecular weight marker (Kaleidoscope Precision Plus Protein Standards, BioRad), with the 20 kDa (solid triangle) and 15 kDa (open triangle) bands indicated. For reference, purified bovine myoglobin (Innovative Research, Novi MI) was loaded into the final three lanes at approximately 1, 3 and 10 ng, as indicated.

FIG. 7 illustrates that myoglobin could be detected in seed from individual transgenic plants that carried T-DNAs from vectors pAG5016 (SEQ ID NO: 48), pAG5019 (SEQ ID NO: 510, and pAG5020 (SEQ ID NO: 56).

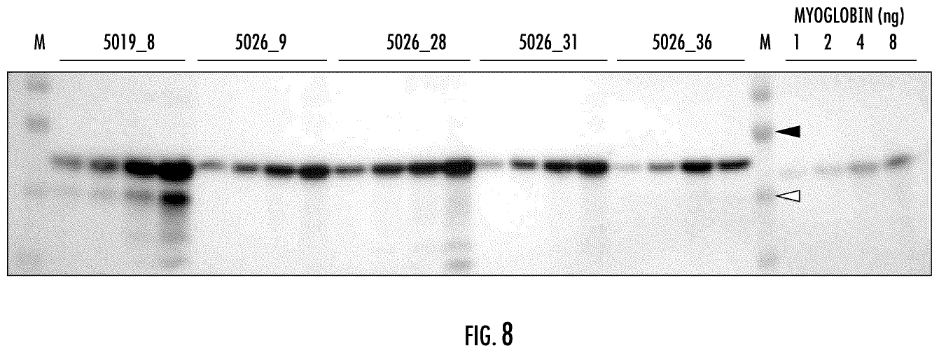

Similarly, myoglobin could be detected in seed from transgenic plants carrying T-DNAs derived from pAG5026, which express the mutant form of bovine myoglobin, BtMYGm. FIG. 8 is a photograph of the Western blot that shows detection of the mutant form of myoglobin in seed from plants that had been transformed with pAG5026. M, molecular weight markers, as in FIG. 7. In subsequent lanes (left to right) were loaded 1, 2, 4 or 8 μl of seed extract (prepared as described above) from pAG5019 candidate #8, which expresses the non-mutated from of myoglobin (see also FIG. 7) and each of four candidates (#9, 28, 31 and 36) transformed with pAG5026, which express the mutated form of myoglobin. For reference, 1, 2, 4 and 8 ng of purified bovine myoglobin were loaded into the final four lanes.

In contrast, little or no myoglobin could be detected from plants transformed with pAG5034, which uses the soybean CG1 promoter. Previous descriptions (See Davis and Lassner, U.S. Patent Application No. 62/429,565, Pub. NO. US 2019/0292217) suggested using the CG1 promoter for expressing heme-containing proteins in plants, including leghemoglobin. However, use of this promoter in corn showed greatly reduced expression levels when compared to the levels obtained using the rice glutelin-1, rice glutelin B-4, maize gamma zein 27, barley Ltp2, or maize A19 promoters. Indeed, many known promoters that are derived from dicotyledonous plants, such as the soy beta-conglycin seed specific promoter and Kunitz tryipson inhibitor promoter, would not be expected to provide high expression levels in monocotyledonous plants such as corn, rice, sorghum, wheat, oats or barley. Furthermore, using these promoters in combination with co-expression of heme biosynthesis enzymes did not significantly increase expression levels.

### Example 15. Evaluation of Extraction Conditions

To determine whether myoglobin might be extracted more efficiently from grain, western blots were used to test the effect of pH on myoglobin extraction.

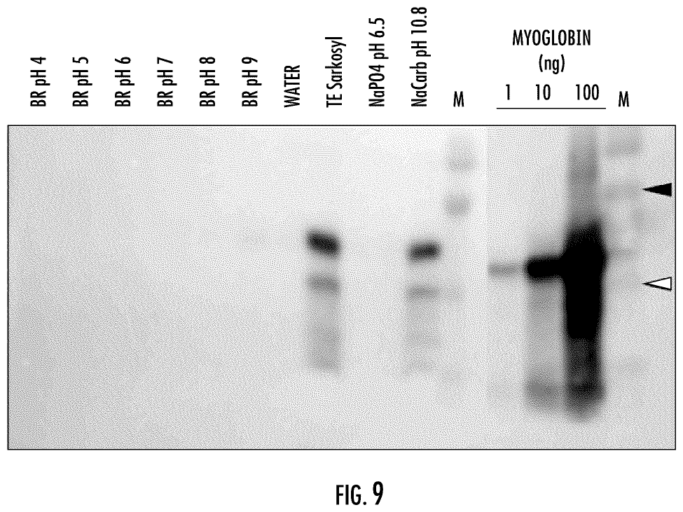

FIG. 9 is a photograph of the Western blot that shows the effect of pH on extraction of myoglobin from corn seed. 250 μl of each of several buffers were used to extract protein from 50 mg ground seed from 5019_8. Approximately 50 μg of total protein (as estimated by Nanodrop spectrophotometer, Thermo Scientific) from each extract was loaded onto a polyacrylamide gel and examined via western blot. Buffers tested: Britton-Robinson (BR) polybuffer (Britton & Robinson, 1931), pH4 to pH9; deionized water; 100 mM Tris, 10 mM EDTA, 1% sarkosyl (TE Sarkosyl); 100 mM sodium phosphate (NaPO4), pH6.5; 30 mM sodium carbonate/bicarbonate (NaCarb), pH10.8. M, molecular weight markers, as in previous figures. 1, 10 or 100 ng of myoglobin, solubilized in 30 mM sodium carbonate/bicarbonate (NaCarb), pH10.8, was loaded for reference. As shown in FIG. 9, changing the pH alone from pH4 to pH9 was insufficient to enable extraction of myoglobin from corn seed. Similarly, water or sodium phosphate buffer (pH 6.5) were unable to extract substantial myoglobin. In contrast, sodium carbonate/bicarbonate buffer (as was used for the examples illustrated in FIGS. 7 and 8, above) and a buffer containing 100 mM Tris, 10 mM EDTA and 1% sarkosyl effectively extracted myoglobin.

To further test the effect of including a detergent in the extraction buffer, samples of purified myoglobin and ground seed samples were extracted in a series of buffers either containing or lacking detergent and examined via western blot.

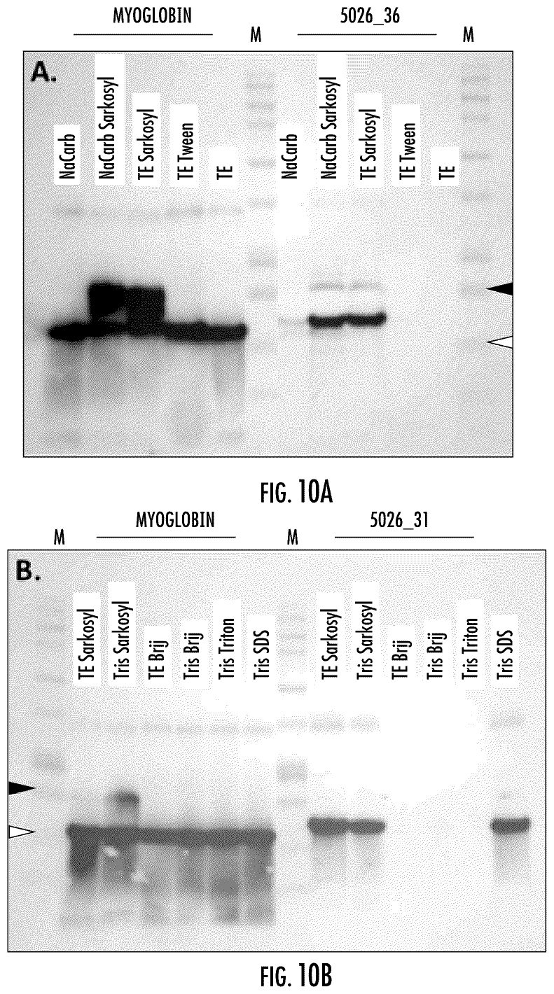

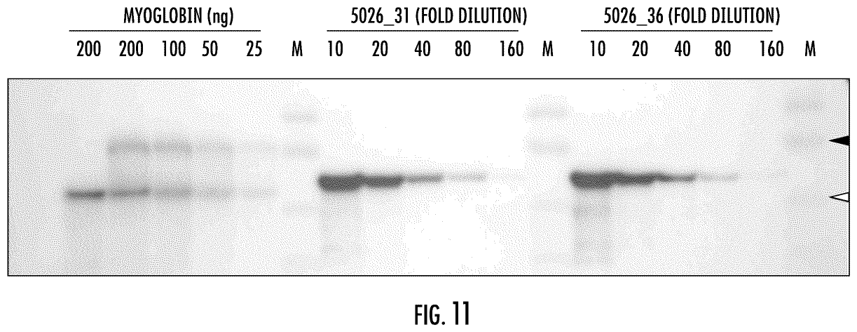

FIGS. 10A-10B are photographs of the Western blots that show the effect of detergent on myoglobin extraction efficiency.

FIG. 10A illustrates the effect the following extraction buffers: NaCarb, 30 mM sodium carbonate/bicarbonate, pH10.8; NaCarb Sarkosyl, 30 mM sodium carbonate/bicarbonate, pH10.8, 1% sarkosyl; TE Sarkosyl, 100 mM Tris 10 mM EDTA, pH8, 1% sarkosyl; TE Tween, 100 mM Tris 10 mM EDTA, pH8, 1% Tween-20; TE, 100 mM Tris 10 mM EDTA, pH8. In this figure, either purified bovine myoglobin (50 mg used per extract; 200 ng loaded from each in lanes 1-5) or ground samples from pAG5026 candidate #36 (approximately 200 mg each) were extracted for 1 hour at room temperature in 1 ml of each of the buffers as follows: NaCarb, 30 mM sodium carbonate/bicarbonate, pH10.8; NaCarb Sarkosyl, 30 mM sodium carbonate/bicarbonate, pH10.8, 1% sarkosyl; TE Sarkosyl, 100 mM Tris 10 mM EDTA, pH8, 1% sarkosyl; TE Tween, 100 mM Tris 10 mM EDTA, pH8, 1% Tween-20; TE, 100 mM Tris 10 mM EDTA, pH8; for seed samples, 10 ul of 20-fold dilutions of each extract was loaded per lane; M, molecular weight markers, as in previous figures.

FIG. 10B illustrates the effect of the following extraction buffers: TE sarkosyl, 100 mM Tris 10 mM EDTA, pH8, 1% sarkosyl; Tris Sarkosyl, 100 mM Tris, pH8, 1% sarkosyl; TE Brij, 100 mM Tris 10 mM EDTA, pH8, 0.03% Brij-35; Tris Brij, 100 mM Tris, pH8, 0.03% Brij-35; Tris Triton, 100 mM Tris, pH8, 0.05% Triton X-100; Tris SDS, 100 mM Tris, pH8, 1% sodium dodecyl sulfate. In this figure, 10 mg samples of purified bovine myoglobin or 100 mg samples of ground seed from pAG5026 candidate #31 were extracted for 1 hour at room temperature in 500 μl of the following buffers: TE sarkosyl, 100 mM Tris 10 mM EDTA, pH8, 1% sarkosyl; Tris Sarkosyl, 100 mM Tris, pH8, 1% sarkosyl; TE Brij, 100 mM Tris 10 mM EDTA, pH8, 0.03% Brij-35; Tris Brij, 100 mM Tris, pH8, 0.03% Brij-35; Tris Triton, 100 mM Tris, pH8, 0.05% Triton X-100; Tris SDS, 100 mM Tris, pH8, 1% sodium dodecyl sulfate; 200 ng of; solubilized myoglobin was loaded per lane in lanes 2-7; for seed samples 10 μl of 20-fold dilutions of each extract was loaded per lane.

As shown in FIGS. 10A and 10B, purified myoglobin was soluble in all of the buffers tested, but some extraction buffers were more effective than others for solubilizing myoglobin from seed samples. While sodium carbonate/bicarbonate buffer extracted myoglobin from seed, the addition of 1% sarkosyl greatly enhanced the amount of myoglobin that could be extracted from seed samples. In contrast 1% Tween-20 did not enhance extraction efficiency (FIG. 10A). Furthermore, neither Brij-35 nor Triton X-100 enhanced myoglobin extraction efficiency at the detergent concentrations tested, although 1% sodium dodecyl sulfate appeared to enhance extraction efficiency as well as sarkosyl (FIG. 10B). The presence of sarkosyl and perhaps sodium dodecyl sulfate caused a portion of the myoglobin to migrate more slowly in both purified and seed-derived myoglobin samples.

### Example 16. Quantitation of Myoglobin Extracted from Seed

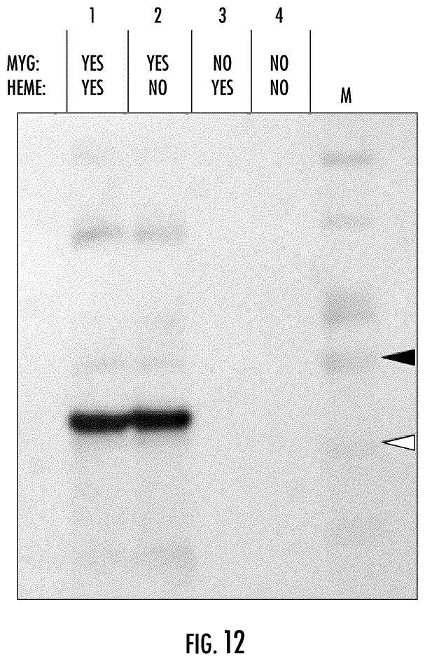

The T-DNAs in progeny from both pAG5026 candidates #31 and #36 segregated in a Mendelian fashion as single-locus insertions. These plants were grown to maturity and self-pollinated. According to Mendelian genetics, it is expected that one quarter of the resulting seed would be homozygous for the respective T-DNA, one half would be hemizygous for the T-DNA, and one quarter would be null for the T-DNA. Samples of 20 seed from self-pollinated ears from each of these candidates were ground to fine powders and 200 mg samples were extracted with 1 ml of 30 mM sodium carbonate/bicarbonate, pH10.8, 1% sarkosyl at room temperature for 1 hour. A dilution series was prepared from each of these extracts, and 10 μl from each dilution was loaded onto a polyacrylamide gel alongside a dilution series of samples of purified bovine myoglobin (10 mg) that had been similarly solubilized in 500 ul of 30 mM sodium carbonate/bicarbonate, pH10.8, 1% sarkosyl. FIG. 11 are photographs that show quantitation of myoglobin in seed from pG5026 candidates #31 and #36. The photograph to the left shows various amounts of purified myoglobin loaded as references; the photograph in the middle shows concentration of myoglobin in diluted samples of the candidate #31; and the photograph to the right shows concentration of myoglobin in diluted samples of the candidate #36. Various amounts of purified myoglobin solubilized either in 30 mM sodium carbonate/bicarbonate, pH10.8 (first lane) or in 30 mM sodium carbonate/bicarbonate, pH10.8, 1% sarkosyl (lanes 2-5) were loaded as references. Note that the presence of sarkosyl caused a portion of the myoglobin to migrate more slowly during electrophoresis, as was seen previously (see FIGS. 10A-10B). 10 μl samples of 10- to 160-fold dilutions of extracts from pAG5026 candidates #31 and #36 were loaded into lanes 7-11 and 13-17, respectively; M, molecular weight markers, as in previous figures.

FIG. 11 shows a western blot of the gel, in which 10 μl from the 40-fold dilutions of both pAG5026 candidates #31 and #36 produced band intensities similar to the 200 ng sample of purified myoglobin. These results indicate that approximately 4 mg of myoglobin could be extracted per gram of seed from both candidates. Since the tested seed were still segregating for the T-DNA, fully homozygous seed derived from these candidates would produce between 8-12 mg of myoglobin per gram of seed (as endosperm maize cells are triploid). Furthermore, when candidates #31 and #36 are crossed, the resulting homozygous seed would produce 16-24 mg of myoglobin per gram of seed.

### Example 17. Supplementation of Myoglobin-Expressing Plants with Genes from the Heme Biosynthetic Pathway

The presence of heme-containing enzymes, such as those involved in carotenoid biosynthesis, indicates that some level of heme is available in corn kernels (Cuttriss, Cazzonelli, Wurtzel, & Pogson, 2011; Proulx, 2007; Saxena et al., 2013). However, it is possible that enhancing heme synthesis in seed tissues might support the accumulation of additional myoglobin. To test this, pAG5029, which carries genes for expressing two key enzymes in the heme biosynthetic pathway (a truncated glutamyl-tRNA reductase and a ferrochelatase), was used to transform maize. Pollen was collected from mature transformants and used to pollinate silks from progeny of pAG5026 candidate #36, which express myoglobin. Individual seed from the resulting cross were screened by PCR for the presence of T-DNAs derived from either pAG5026 or pAG5029, then ground to a fine powder. Myoglobin was extracted from the powder samples with 100 mM Tris 10 mM EDTA, pH8, 1% sarkosyl (at a ratio of 10 ml buffer per gram of powder) at room temperature for 1 hour. The extracts were diluted 20-fold in extraction buffer, and 10 ul of each sample was loaded onto a polyacrylamide gel. Myoglobin was then detected via western blot.

FIG. 12 is a photograph that shows the effect of genes encoding heme biosynthetic enzymes on myoglobin accumulation. Individual seed from a cross between pAG5026 candidate #36 and a pAG5029 candidate were screened for the presence of the T-DNA from pAG5026 carrying the myoglobin genes (“myg”) and/or the T-DNA from pAG5029 carrying the genes for the truncated glutamyl-tRNA reductase and the ferrochelatase (“heme”). Seed 1 carried both T-DNAs, seed 2 carries only the myoglobin T-DNA, seed 3 carries only the heme T-DNA, and seed 4, which was taken from a wild type (untransformed) plant, carries neither T-DNA. M, molecular weight markers, as in previous figures.

As shown in FIG. 12, the presence of the genes from the heme pathway did not appear to enhance myoglobin accumulation in seed. This is a surprising result, given previous disclosures (such as Davis and Lassner, U.S. Patent Application No. 62/429,565, Pub. NO. US 2019/0292217) that taught co-expression of heme biosynthesis genes such as glutamyl-tRNA reductase and ferochelatase are necessary to achieve high expression levels of heme-proteins such as myoglobin.

While myoglobin, glutamyl-tRNA reductase, and ferrochelatase genes can all be delivered as a molecular combination in the same T-DNA, transforming them independently and combining them through crossing of transgenic plants can be advantageous. Although different promoters can be used to help enable different transgene expression levels, allowing the myoglobin expression cassette to integrate into the corn genome at a different location from the glutamyl-tRNA reductase and ferrochelatase expression cassettes allows combination of the genes at any relative expression level through crossing. For example, since the heme biosynthesis genes are only needed at levels to catalyze the formation of heme biosynthesis, it may not be necessary to express them at a high-level, similar to myoglobin. As an example if myoglobin is expressed at 20 mg per gram of seed, but glutamyl-tRNA reductase and ferrochelatase only need to be expressed at a level of 3 μg per gram of seed to provide enough heme for myoglobin accumulation, such a combination can be achieved by crossing events that have the desired expression level. We made multiple crosses of myoglobin expressing plants with plants expressing glutamyl-tRNA reductase and ferrochelatase, resulting in plants with a variety of different expression ratios (that is, different ratios of myoglobin accumulation relative to glutamyl-tRNA reductase and ferrochelatase).

### REFERENCES

Becana, M., Yruela, I., Sarath, G., Catal An, P., & Hargrove, M. S. (2020). Plant hemoglobins: a journey from unicellular green algae to vascular plants. New Phytologist, 227, 1618-1635. https://doi.org/10.1111/nph.16444
Britton, H. T. S., & Robinson, R. A. (1931). Universal buffer solutions and the dissociation constant of veronal. Journal of the Chemical Society (Resumed), 1456-1462. https://doi.org/https://doi.org/10.1039/JR9310001456
Cuttriss, Abby J., Christopher I. Cazzonelli, Eleanore T. Wurtzel, and Barry J. Pogson. 2011. Carotenoids. Advances in Botanical Research. Vol. 58. https://doi.org/10.1016/B978-O-12-386479-6.00005-6.
Doyle J. J., Schuler M. A., Godette W. D., Zenger V., Beachy R. N. (1986). The Glycosylated Seed Storage Proteins of Glycine max and Phaseolus vulgaris. The Journal of Biological Chemistry, 261 (20), 9228-9238.
Hilton J J C. (1953). The distribution of protein in the maize kernel in comparison with that in wheat. Cereal Chem. 30, 441-445.
Kalla R., Shimamoto K., Potter R., Nielsen P. S., Linnestad C., Olsen O.-A. (1994). The promoter of the barley aleurone-specific gene encoding a putative 7 kDa lipid transfer protein confers aleurone cell-specific expression in transgenic rice. The Plant Journal 6(6), 849-860.
Ndolo V. U., Beta T. (2013). Distribution of carotenoids in endosperm, germ, and aleurone fractions of cereal grain kernels. Food Chem. 139 (1-4): 663-71.
Nishizawa K., Maruyama N., Satoh R., Fuchikami Y., Higasa T., Utsumi S. (2003). A C-terminal sequence of soybean b-conglycinin α′ subunit acts as a vacuolar sorting determinant in seed cells. The Plant Journal, 34, 647-659.
Olsen O.-A., Kalla R. (1996). Ltp2 promoter having aleurone-tissue-specific activity. U.S. Pat. No. 5,525,716.
Proulx, Amy Katheryn. 2007. “Diversified Strategies for Improving Iron Bioavailability of Maize.” Iowa State University.
Royo J., Gómez E., Sellam O., Gerentes D., Paul W., Hueros G. (2014). Two maize END-lorthologs, BETL9 and BETL9like, are transcribed in a non-overlapping spatial pattern on the outer surface of the developing endosperm. Front Plant Sci. 5, 180.
Saxena, Akansha, Priyanka Singh, Dharmendra K. Yadav, Pooja Sharma, Sarfaraz Alam, Feroz Khan, Sanjog T. Thul, Rakesh K. Shukla, Vikrant Gupta, and Neelam S. Sangwan. 2013. “Identification of Cytochrome P450 Heme Motif in Plants Proteome.” Plant OMICS 6 (1): 1-12.
Uppal, S., Salhotra, S., Mukhi, N., Zaidi, F. K., Seal, M., Dey, S. G., . . . Kundu, S. (2015). Significantly enhanced heme retention ability of myoglobin engineered to mimic the third covalent linkage by nonaxial histidine to heme (vinyl) in Synechocystis hemoglobin. Journal of Biological Chemistry, 290(4), 1979-1993. https://doi.org/10.1074/jbc.M114.603225
Vothknecht U. C., Kannangara C. G., von Wettstein D. (1998). Barley glutamyl tRNAGlu reductase: mutations affecting haem inhibition and enzyme activity. Phytochemistry, 47 (4): 513-9.
Wolf M. J., Cutler H. C., Zuber M. S., Khoo U. (1972). Maize with Multilayer Aleurone of High Protein Content. Crop Sci. 12 (4): 440-442.
Woodson J. D., Perez-Ruiz J. M., Chory J. (2011). Heme Synthesis by Plastid Ferrochelatase I Regulates Nuclear Gene Expression in Plants. Current Biology, 21, 897-903.
Zhai S., Xia X., He Z. (2016). Carotenoids in Staple Cereals: Metabolism, Regulation, and Genetic Manipulation. Front Plant Sci. 7: 1197.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A transgenic plant or tissue thereof comprising a synthetic polynucleotide encoding at least one animal-derived protein, wherein the animal-derived protein is selected from the group consisting of: a myoglobin, hemoglobin, chymosin, and casein proteins, the transgenic plant is a monocotyledonous plant, the tissue thereof is a grain and the at least one animal-derived protein is expressed in the grain at a level in the range from 0.01 mg to 24.0 mg of the animal-derived protein per gram of grain.

2. (canceled)

3. The transgenic plant or tissues thereof of claim 1, wherein the monocotyledonous plant is selected from the group consisting of: corn, rice, wheat, oat, barley and millet.

4-6. (canceled)

7. The transgenic plant or tissues thereof of claim 3, wherein the monocotyledonous plant is corn, and at least one animal-derived protein is expressed in the corn grain.

8-9. (canceled)

10. The transgenic plant or tissues thereof of claim 7, wherein the animal-derived protein is the myoglobin protein.

11-13. (canceled)

14. The transgenic plant or tissues thereof of claim 1, wherein the animal-derived protein is the myoglobin protein and the nucleic acid encoding the myoglobin protein comprises a sequence with at least 90% identity to a reference sequence of SEQ ID NO: 32, or 78.

15. The transgenic plant or tissues thereof of claim 1, wherein the animal-derived protein is the myoglobin protein and the myoglobin protein comprising an amino acid sequence with at least 95% identity to a reference sequence selected from the group consisting of: SEQ ID NOS: 1-3, 20, 40 and 58.

16-31. (canceled)

32. The transgenic plant or tissues thereof of claim 1 wherein the synthetic polynucleotide comprises a nucleic acid encoding a hemoglobin protein.

33. The transgenic plant or tissues thereof of claim 32, wherein the nucleic acid encoding the hemoglobin protein comprises a sequence with at least 90% identity to a reference sequence of SEQ ID NO: 33.

34. The transgenic plant or tissues thereof of claim 32, wherein the hemoglobin protein comprises an amino acid sequence with at least 95% identity to a reference sequence selected from the group consisting of: SEQ ID NOS: 4-6, 21, and 41.

35-37. (canceled)

38. The transgenic plant or tissues thereof of claim 1, wherein the synthetic polynucleotide comprises a nucleic acid encoding a chymosin protein.

39. The transgenic plant or tissues thereof of claim 38, wherein the nucleic acid encoding the chymosin protein comprises a sequence with at least 90% identity to a reference sequence of SEQ ID NO: 31.

40. The transgenic plant or tissues thereof of claim 38, wherein the chymosin protein comprises an amino acid sequence with at least 95% identity to a reference sequence selected from the group consisting of: SEQ ID NOS: 14, 19, and 39.

41. The transgenic plant or tissues thereof of claim 1, wherein the synthetic polynucleotide comprises a nucleic acid encoding a casein protein.

42. The transgenic plant or tissues thereof of claim 41, wherein the nucleic acid encoding the casein protein comprises a sequence with at least 90% identity to a reference sequence selected from the group consisting of SEQ ID NOS: 27-30.

43. The transgenic plant or tissues thereof of claim 41, wherein the casein protein comprises an amino acid sequence with at least 95% identity to a reference sequence selected from the group consisting of: SEQ ID NOS: 10-13, 15-18, and 35-38.

44-46. (canceled)

47. The transgenic plant or tissues thereof of claim 1, wherein the animal-derived protein is the myoglobin protein, and the synthetic nucleic acid encoding the myoglobin protein is included in the expression cassette that comprises a sequence with at least 90% identity to a reference sequence selected from the group consisting of: SEQ ID NOs: 48, 51-52, 64-66, 70, 72, and 74-77.

48-58. (canceled)

59. A plant-based meat composition comprising at least one of the transgenic plants or tissues thereof of claim 1, wherein the animal-derived protein is the myoglobin, hemoglobin or a combination thereof.

60-64. (canceled)

65. A plant-based cheese composition comprising at least one of any of the transgenic plants or tissues thereof of claim 1, wherein the animal-derived protein comprises the chymosin, casein or a combination thereof.

66-69. (canceled)

70. A process for isolating at least one animal-derived protein from a transgenic plant or tissues thereof of claim 1.

71. The process of claim 70, wherein the isolation is a modified wet milling or dry milling process.

72-82. (canceled)

83. The process of claim 71, wherein the isolation occurs by extraction of the at least one animal-derived protein from the corn gluten feed, or from the corn gluten meal.

84-89. (canceled)
